(19) United States
(12) Patent Application Publication
(10) Pub. No.: US 2018/0360856 A1
(43) Pub. Date: Dec. 20, 2018

(54) ANTI-BACTERIAL COMPOUNDS

(71) Applicant: Auspherix Limited, Stevenage (GB)

(72) Inventors: Ian HOLMES, Stevenage (GB); Alan NAYLOR, Royston (GB); Dagmar ALBER, Markyate (GB); Jonathan Raymond POWELL, Cambridge (GB); Meriel Ruth MAJOR, Cambridge (GB); Gabriel NEGOITA-GIRAS, Cambridge (GB); Daniel Rees ALLEN, Cambridge (GB); Lucie Juliette GUETZOYAN, Cambridge (GB); Nigel Paul KING, Stevenage (GB)

(73) Assignee: Auspherix Limited, Steveage (GB)

(21) Appl. No.: 15/781,094

(22) PCT Filed: Dec. 2, 2016
(86) PCT No.: PCT/EP2016/079679

§ 371 Date: Jun. 1, 2018

(30) Foreign Application Priority Data
Dec. 2, 2015 (GB) 1521238.4

(51) Int. Cl. A61K 31/675; A61P 31/04

(52) U.S. Cl. CPC A61K 31/675; A61K 31/28; A61P 31/04

(57) ABSTRACT

A compound of Formula (II): for use in the prevention or treatment of a bacterial infection.

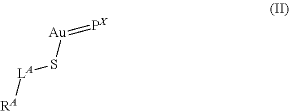

The present invention relates to gold(I)-phosphine compounds, and their use as inhibitors of growth of Gram-positive and/or Gram-negative bacteria. The present invention also relates to using such compounds for the prevention and/or treatment of bacterial infection.

The global rise of bacteria and other microorganisms resistant to antibiotics and antimicrobials in general, poses a major threat. Deployment of massive quantities of antimicrobial agents into the human ecosphere during the past 60 years has introduced a powerful selective pressure for the emergence and spread of antimicrobial-resistant bacterial pathogens. The World Health Organization has highlighted antimicrobial resistance (AMR) as an issue of global concern in 2014. AMR is now present in all parts of the world with the incidence of antibiotic resistance (ABR) in bacteria that cause common infections (e.g. pneumonia, bloodstream infections and urinary tract infections) rendering many historically efficacious antibiotics ineffective. Of particular concern are hospital-acquired infections caused by highly resistant bacteria such as the ESKAPE pathogens (Enterococcus faecium, Staphylococcus aureus, Klebsiella pneumoniae, Acinetobacter baumannii, Pseudomonas aeruginosa, and Enterobacter species), Escherichia coli, Coagulase-negative staphylococci and Clostridium difficile. Additionally, failure of last resort third-generation cephalosporins for the treatment of gonorrhea has now been reported in 10 countries raising the possibility that gonorrhea may soon become untreatable in the absence of new antibacterial agents.

The biological activity of gold(I) and gold(III) complexes has been studied historically and salts of both have been demonstrated to possess antimicrobial activity against a range of pathogens. Gold(I) complexes have historically been reported as having antibacterial activity against Gram positive organisms. (Glišić, B.D. & Djuran M.I., Dalton Trans., 2014, 43, 5950-5969).

Gold(I) is a soft Lewis acid and preferentially complexes with soft donor atoms such as sulfur, selenium and phosphorous. Examples of such complexes used clinically include gold thiomalate, aurothioglucose and auranofin:

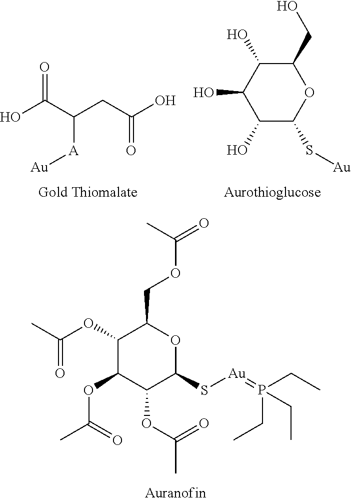

Auranofin, a second generation orally bioavailable gold(I) based treatment for rheumatoid arthritis (RA), has been identified as inhibiting the in vitro growth of S. aureus (Oxford strain) with an MIC of 0.6-0.9 μg/mL and V. cholerae with an MIC of 2.5 μg/mL. These observations reinforce multiple literature reports of the antimicrobial activity of auranofin and other gold(I) compounds against a range of bacterial pathogens (Aguinagalde L., et al., J. Antimicrob. Chemother., 2015, 70(9), 2608-2617; Harbut, M B, et al., PNAS, 2015, 112(14), 4453-4458; Madeira, J M., Inflammopharmacology, 2012, 20, 297-306; Jackson-Rosario, S, J. Biol. Inorg. Chem., 2009, 14(4), 507-519; Novelli, F., Farmaco, 1999, 54, 232-236; Shaw, C F, Chem Rev., 1999, 99(9), 2589-2600; Rhodes, M D, J. Inorg. Biochem., 1992, 46, 129-142 and Fricker, S P, Transition Met. Chem., 1996, 21, 377-383). Auranofin has not been shown to have any significant activity against the majority of Gram negative bacteria.

Co-pending applications PCT/GB2015/051551 and PCT/GB2015/051550 describe certain gold(I) phosphine compounds and their use as inhibitors of growth of Gram-positive and/or Gram-negative bacteria.

A first aspect of the present invention provides a compound according to Formula (I):

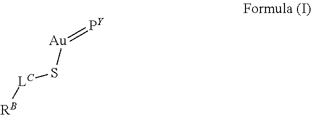

wherein

P^{Y} is independently selected from the group consisting of (P1), (P2) and (P3);

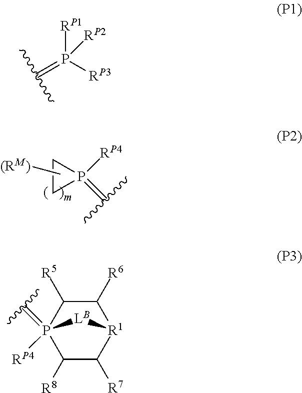

wherein
—L^{C}— is methylene, ethylene or is absent;
R^{P1} and R^{P2} are each independently selected from methyl;
when —L^{C}— is absent R^{P3} is selected from the group consisting of cyclopentyl, t-butyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;
when —L^{C}— is methylene or ethylene R^{P3} is selected from the group consisting of methyl and ethyl,
4-membered or 5-membered heterocycloalkyl group linked to phosphorus via a carbon atom in the ring, including a single heteroatom independently selected from NR^{Z}, O and S,
   —CF₃, —CH₂CF₃, —CH₂CF₂H, —CH₂CH₂OR^{PB},
CH₂Q and —(CH₂)₂Q;

wherein Q is a O₅₋₆ heteroaryl group, optionally substituted with one or more groups R^{PA};

R^{P4} is selected from methyl and ethyl;

m is an integer selected from 1, 2 or 3;

R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;

when —L^{B}— is present, R^{P4} is absent and R¹ is selected from N, CH and CR^{PC};

when —L^{B}— is absent, R¹ is selected from the group consisting of O,
NR^{Z},
SO₂,
CH₂, CHF, CF₂ and CHR^{PC};

wherein R^{Z} is selected from the group consisting of
—H, —C₁₋₃alkyl, —COC₁₋₃alkyl and —SO₂C₁₋₃alkyl;

R⁵ and R⁸ are each independently selected from —H and —R^{PC};

R⁶ and R⁷ are each independently selected from —H and —R^{PC};

wherein R^{PC} is selected from the group consisting of
C₁₋₃alkyl, optionally substituted with one or more groups R^{PD};

wherein R^{PA} is selected from the group consisting of
linear or branched C₁₋₃alkyl, C₂₋₆alkenyl or C₂₋₆alkynyl optionally substituted with one or more groups R^{AL},
—F, —Cl, —Br, —CN
—OH, —OR^{PE},
—CF₃, —CF₂H,
—COR^{PE},
—CH₂OH, —CH₂OR^{PE},
—COOH, —COOR^{PE}, —CONH₂, —CONHR^{PE}, —CONR^{PE}₂,
—OCOR^{PE}, —OCONH₂, —OCONHR^{PE}, —OCONR^{PE}₂,
—NH₂, —NHR^{PE}, —NR^{PE}₂,
—SO₂NH₂, —SO₂NHR^{PE}₂, —SO₂NR^{PE}₂,
—SO₂R^{PE},
—NHCOH, —NHCOR^{PE}, —NR^{PE}COH and —NR^{PE}COR^{PE};
   and R^{PB} is selected from the group consisting of
linear or branched C₁₋₆alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

R^{PE} is selected from
linear or branched C₁₋₄alkyl optionally substituted with one or more groups R^{PD};

and R^{PD} is selected from the group consisting of
F,
OH and OC₁₋₃alkyl.

R^{B} is independently selected from the groups (A1) to (A5)

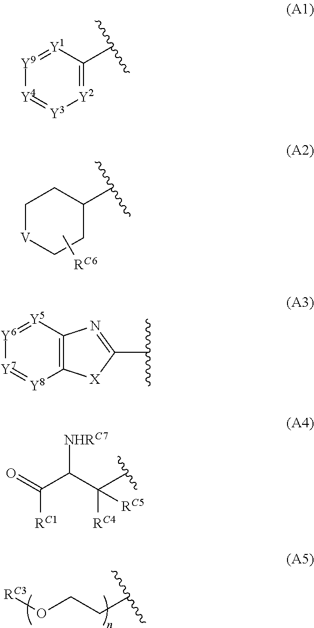

wherein

each of Y¹, Y², Y³, Y⁴ and Y⁹ is independently selected from CH or N; wherein at least three of Y¹, Y², Y³, Y⁴ and Y⁹ are independently CH;

V is independently selected from O, CH—OR^{O1}, N—CO—R^{C3}, N—CO—NHR^{C8}, N—SO₂—R^{C8}, N—CO₂—R^{C2} and N—R^{N2};

one of Y⁵, Y⁶, Y⁷ and Y⁸ is selected from CH and N, and the others are CH;

X is independently selected from NH, S and O;

R^{C1} is selected from O—R^{O2} or NHR^{N1};

R^{O1} is selected from H and C₁₋₃ unbranched alkyl;

R^{O2} is selected from H and C₁₋₃ unbranched alkyl;

R^{N1} is selected from H and C₁₋₃ unbranched alkyl;

R^{N2} is C₁₋₃ unbranched alkyl;

R^{C2} and R^{C8} are each independently selected from C₁₋₃ unbranched alkyl and C₃₋₄ branched alkyl;

R^{C3} is selected from C₁₋₃ unbranched alkyl and C₂H₄CO₂H;

R^{C4} is either H or Me;

R^{C5} is either H or Me;

R^{C6} represents one or two optional methyl substituents;

R^{C7} is selected from —H and —COCH₃; and

n is an integer selected from 2 to 8;

and pharmaceutically acceptable salts, solvates and hydrates thereof.

In some embodiments when —L^{C}— is absent R^{P3} is selected from the group consisting of
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q.

A second aspect of the present invention provides a compound of formula (I) for use in the prevention or treatment of a bacterial infection. The second aspect of the invention also provides the use of a compound of formula (I) in the manufacture of a medicament for the treatment and/or prevention of a bacterial infection. The first aspect of the invention further provides the treatment of a human or animal patient afflicted with a bacterial infection, comprising administering to said patient an effective amount of a pharmaceutical composition containing a compound of formula (I).

The second aspect may also relate to the treatment of fungal infection, e.g. by providing a compound of formula (I) for use in the prevention or treatment of a fungal infection.

A third aspect of the present invention provides a compound of Formula (II):

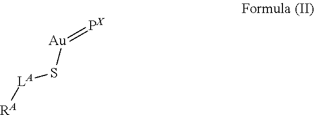

for use in the prevention or treatment of a bacterial infection wherein P^{X} is selected from the group consisting of (P1), (P2) and (P3);

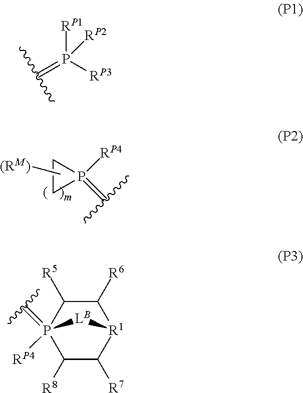

wherein

R^{P1} and R^{P2} are each independently selected from methyl, ethyl, isopropyl and phenyl;

R^{P3} is selected from the group consisting of
methyl and ethyl ,
isopropyl,
cyclopentyl,
t-butyl,
phenyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;

wherein Q is a C₅₋₆ heteroaryl group, optionally substituted with one or more groups R^{PA};

R^{P4} is selected from methyl and ethyl;

m is an integer selected from 1, 2 or 3;

R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;

—L^{B}— is methylene, ethylene or is absent;

when —L^{B}— is present, R^{P4} is absent and R¹ is selected from N, CH and CR^{PC};

when —L^{B}— is absent, R¹ is selected from the group consisting of
O,
NR^{Z},
SO₂,
CH₂, CHF, CF₂ and CHR^{PC};

wherein R^{Z} is selected from the group consisting of
—H, —C₁₋₃alkyl, —COC₁₋₃alkyl and —SO₂C₁₋₃alkyl;

R⁵ and R⁸ are each independently selected from —H and —R^{PC};

R⁶ and R⁷ are each independently selected from —H and —R^{PC};

wherein R^{PC} is selected from the group consisting of
C₁₋₃alkyl, optionally substituted with one or more groups R^{PD};

wherein R^{PA} is selected from the group consisting of
linear or branched C₁₋₃alkyl, C₂₋₆alkenyl or C₂₋₆alkynyl optionally substituted with one or more groups R^{AL},
—F, —Cl, —Br, —CN
—OH, —OR^{PE},
CF₃, —CF₂H,
—COR^{PE},
—CH₂OH, —CH₂OR^{PE},
—COOH, —COOR^{PE}, —CONH₂, —CONHR^{PE}, —CONR^{PE}₂,
—OCOR^{PE}, —OCONH₂, —OCONHR^{PE}, —OCONR^{PE}₂,
—N H₂, —NHR^{PE}, —NR^{PE}₂,
—SO₂NH₂, —SO₂NHR^{PE}₂, —SO₂NR^{PE}₂,
—SO₂R^{PE},
—NHCOH, —NHCOR^{PE}, —NR^{PE}COH and —NR^{PE}COR^{PE};
   and R^{PB} is selected from the group consisting of
linear or branched C₁₋₃alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

R^{PE} is selected from
linear or branched C₁₋₄alkyl optionally substituted with one or more groups R^{PD};

and R^{PD} is selected from the group consisting of
F,
OH and OC₁₋₃alkyl;

—L^{A}— is selected from
methylene optionally substituted with one or two groups R^{1A1},
ethylene optionally substituted with one or more groups R^{1A1}, and
a single bond;

R^{A} is selected from the group consisting of
(i) 5-membered heteroaromatic groups containing at least one heteroatom selected from N, O and S optionally C-substituted with one or more groups R^{A1}, and optionally N-substituted with one or more groups R^{NA1},
(ii) 6-membered aromatic groups or heteroaromatic groups containing 1 to 3 N atoms, substituted with one or more groups R^{A1},
(iii) 8- to 10-membered bicyclyl or heterobicyclyl groups with the proviso that R^{A} is not selected from the group (A3) or the groups (X3a) to (X3b)

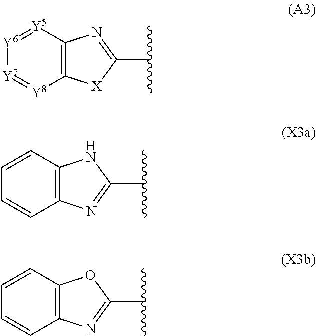

wherein one of Y⁵, Y⁶, Y⁷ and Y⁸ is selected from CH and N, and the others are
CH; and X is independently selected from NH, S and O; and
(iv) the groups (C1) to (C6)

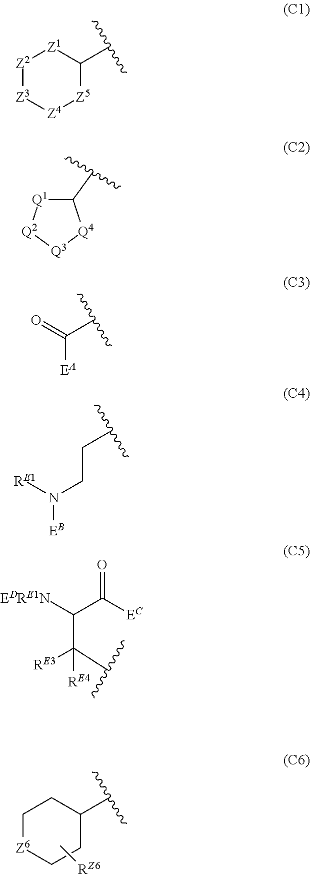

with the proviso that R^{A} is not the group (C3) when L is a single bond;

Z³ is selected from the group consisting of CH₂, CHR^{AL} and CR^{AL}₂;

one of Z¹, Z², Z⁴ and Z⁵ is selected from the group consisting of
CH₂, CHR^{AL}, CR^{AL}₂,
O,
NH, NR^{A2},
N(CO—R^{A2}), N(CO—NHR^{A2}), N(SO₂—R^{A2}) and N(CO₂—R^{A4});

the remainder of Z¹, Z², Z⁴ and Z⁵ are independently selected from the group consisting of
CH₂, CHR^{AL}, CR^{AL}₂, and
O;

with the provisos that the ring contains 0 or 1 oxygen atoms, that nitrogen atoms cannot be in a 1,2 or 1,3 relationship to each other, and that when Z¹ or Z⁵ is N, L cannot be a single bond;

one of Q¹ to Q⁴ is selected from the group consisting of
O,
NH, NR^{A2},
CH₂, CHR^{AL} and CR^{AL}₂,
N—CO—R^{A2}, N—CO—NHR^{A2}, N—SO₂—R^{A2} and N—CO₂—R^{A4}
   the remainder of Q¹ to Q⁴ are independently selected from the group consisting of
NH, NR^{A2},
CH₂, CHR^{AL} and CR^{AL}₂;

with the proviso that the ring contains 0 or 1 oxygen atoms, that the ring contains 0 or 1 nitrogen atoms, and that when Q¹ or Q⁴ is N, L cannot be a single bond;

E^{A} is selected from the group consisting of
—O—R^{A2},
—NH—R^{A2},
—NR^{A2}₂,
—NR^{EA1}-E^{A1}-COR^{EA2} and —NR^{EA1}-E^{A2}-E^{A3}-COR^{EA2},

wherein E^{A1}, E^{A2} and E^{A3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EA1}- and —COR^{EA2} groups represent terminals of the alpha or pendent functionality of the amino acids respectively;

wherein the amino acid residues Asp and Glu may form amide bonds from either the alpha or pendent carboxylic acid functionality;

when E^{A1} is Pro, R^{EA1} is absent, otherwise R^{EA1} is R^{E1};

when E^{A2} is Pro, R^{EA1} is absent, otherwise R^{EA1} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

and when E^{A2} and E^{A3} are present and E^{A3} is not Pro the nitrogen of the amide bond between E^{A2} and E^{A3} may be optionally substituted with R^{E1};

R^{EA2} is selected from —OR^{E7}, —NH₂, —NHR^{A2} and —NR^{A2}R^{E1};

R^{E1} is selected from H and linear or branched C₁₋₃alkyl;

E^{B} is selected from
E^{BA}, —CO-E^{B1}-NR^{EA}R^{E2} and —CO-E^{B2}-E^{B3}-NR^{EB}R^{E2},

wherein E^{B1}, E^{B2} and E^{B3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —CO—, —NR^{EA}R^{E2} and —NR^{EB}R^{E2} groups represent terminals of the alpha or pendent functionality of the amino acids;

wherein the amino acid residues Asp and Glu may form amide bonds from either the alpha or pendent carboxylic acid functionality

when E^{B1} is Pro, R^{EA} is absent, otherwise R^{EA} is R^{E1};

when E^{B3} is Pro, R^{EB} is absent, otherwise R^{EB} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{B2} and E^{B3} are present and E^{B2} is not Pro the nitrogen of the amide bond between E^{B2} and E^{B3} may be optionally substituted with R^{E1};

when E^{B} is E^{BA}, R^{E1} and E^{BA} together with the nitrogen atom to which they are attached form a group selected from
5- or 6-membered saturated heterocyclyl optionally substituted with one or more groups R^{AL}, and
5- or 6-membered heteroaryl optionally substituted with one or more groups R^{A1};

E^{C} is selected from
—OH,
—OR^{A2}
—NH₂, NHR^{A2}, NR^{A2}2 and
—NR^{EC1}-E^{C1}-COR^{EC2}

wherein E^{C1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EC1}— and —COR^{EC2} groups represent terminals of the alpha or pendent functionality of the amino acids;

wherein the amino acid residues Asp and Glu may form amide bonds from either the alpha or pendent carboxylic acid functionality;

when E^{C1} is Pro, R^{EC1} is absent, otherwise R^{EC1} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

R^{EC2} is selected from —OR^{E9}, —NH₂, —NHR^{A2} and —NRA₂RE^{E1};

R^{E3} and R^{E4} are independently selected from —H and —CH³;

when R^{E1} is H and E^{C} is —OC₁₋₃alkyl, —NH₂ or —NHC₁₋₃alkyl, E^{D} is selected from
—H, and
—CO-E^{D1}-NR^{ED}R^{E6}
   otherwise, E^{D} is selected from
—R^{E5}, and
—CO-E^{D1}-NR^{ED}R^{E6};

wherein E^{D1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{ED}R^{E6}- and —CO— groups represent terminals of the alpha or pendent functionality of the amino acids;

wherein the amino acid residues Asp and Glu may form amide bonds from either the alpha or pendent carboxylic acid functionality;

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

when E^{D1} is Pro, R^{ED} is absent, otherwise R^{ED} is R^{E1};

R^{E2}, R^{E5} and R^{E6} are independently selected from —H and —COCH₃;

R^{E7}, R^{E8} and R^{E9} are each independently selected from —H and —R^{A2};

Z⁶ is selected from N—CO—R^{A2}, N—CO—NHR^{A2}, N—SO₂—R^{A2};

R^{Z6} is one or two optional methyl substituents;

R^{A1} is selected from the group consisting of
linear or branched C₁₋₆alkyl, C₂₋₆alkenyl or C₂₋₆alkynyl optionally substituted with one or more groups R^{AL},
—F, —CI, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
   —NH₂, —NHR^{A2}, —NR^{A2}₂,
—SO₂NH₂, —SO₂NHR^{A2}₂, —SO₂NR^{A2}₂,
   —SO₂R^{A2},
—NHCOH, —NHCOR^{A2}, —NR^{A2}COH and —NR^{A2}COR^{A2};

R^{A2} is selected from the group consisting of
linear or branched C₁₋₆alkyl, C₂₋₆alkenyl or C₂₋₆alkynyl optionally substituted with one or more groups R^{AT}, wherein the alkyl chain is optionally interrupted by
one or more atoms selected from O and S,
OC₁₋₆alkyl;
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

where N is substituted by 2 R^{A2} groups, the N and the R^{A2} groups may together form a N-containing C₅₋₆ heterocycloalkyl group, which may be substituted by methyl;

R^{NA1} is selected from linear or branched C₁₋₃alkyl;

R^{1A1} is selected from linear or branched unsubstituted C₁₋₃alkyl;

R^{A3} is selected from H and unbranched unsubstituted C₁₋₃alkyl;

R^{A4} is selected from linear or branched unsubstituted C₁₋₄aalkyl;

R^{AL} is selected from the group consisting of
—F, —CN
—OH, —OR^{A2},
—CF₃, —CF₂H,
COR^{A2},
COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2}; and

wherein R^{AR} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{1A1},
—CF₃, —CF₂H,
—COR^{1A1},
—CH₂OH, —CH₂OR^{1A1}, —CHR^{1A1}OH, —CHR^{1A1}OR^{1A1}
—COOH, —COOR^{1A1}, —CONH₂, —CONHR^{1A1}, —CONR^{1A1}₂,
—OCOR^{1A1}, —OCONH₂, —OCONHR^{1A1}, —OCONR^{1A1}₂,
—NH₂, —NHR^{1A1}, —NR^{1A1}₂,
—SO₂NH₂, —SO₂NHR^{1A1}₂, —SO₂NR^{1A1}₂,
—SO₂R^{1A1},
—NHCOH, —NHCOR^{1A1}, —NR^{1A1}COH and —NR^{1A1}COR^{1A1};

R^{AT} is selected from the group consisting of
—F, —CN
—OH, —OC₁₋₃alkyl,
—CF₃, —CF₂H,
—COC₁₋₃alkyl,
—COOH, —COOC₁₋₃alkyl, —CONH₂, —CONHC₁₋₃alkyl, —CON(C₁₋₃alkyl)₂,
—OCOC₁₋₃alkyl, —OCONH₂, —OCONHC₁₋₃alkyl, —OCON(C₁₋₃ 3alkyl)₂,
—NH₂, —NHC₁₋₃alkyl, —N(C₁₋₃alkyl)₂,
—SO₂NH₂, —SO₂NH(C₁₋₃alkyl)₂, —SO₂N(C₁₋₃alkyl)₂,
—SO₂(C₁₋₃alkyl),
—NHCOH, —NHCO(C₁₋₃alkyl), —N(C₁₋₃alkyl)COH and —N(C₁₋₃alkyl)CO(C₁₋₃alkyl);
   and pharmaceutically acceptable salts, solvates and hydrates thereof.

In the third aspect, R^{A2} may be selected from the group consisting of
linear or branched C₁₋₆alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

R^{P1} and R^{P2} may each be independently selected from methyl;

and R^{P3} may be selected from the group consisting of
methyl and ethyl ,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q.

In some embodiments, where N is substituted by 2 R^{A2} groups, the N and the R^{A2} groups may together form a N-containing C₅₋₆ heterocycloalkyl group which is optionally substituted with one or two groups selected from linear unsubstituted C₁₋₆ alkyl.

The third aspect may also relate to the treatment of fungal infection, e.g. by providing a compound of formula (I) for use in the prevention or treatment of a fungal infection.

The third aspect of the invention also provides the use of a compound of formula (I) in the manufacture of a medicament for the treatment and/or prevention of a bacterial infection. The first aspect of the invention further provides the treatment of a human or animal patient afflicted with a bacterial infection, comprising administering to said patient an effective amount of a pharmaceutical composition containing a compound of formula (I).

In the second and third aspects, the bacterial infection prevented and/or treated may be infection by one or more Gram-positive bacteria. The bacterial infection prevented and/or treated may be infection by one or more Gram-negative bacteria. In the second and third aspect, the bacterial infection prevented and/or treated may be infection by one or more multi-drug resistant bacteria.

Compounds of the present invention may also be used to treat conditions by interaction with, e.g. binding to, thioredoxin reductase (TrxR), glutathione peroxidase (GSPx), IkB kinase (IKK) complex, cathepsins and type I iodothyronine deiodinase.

A fourth aspect of the present invention provides a compound of Formula (II):

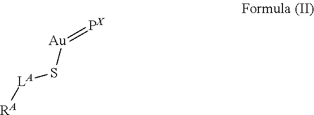

wherein P^{X} is selected from the group consisting of (P1), (P2) and (P3):

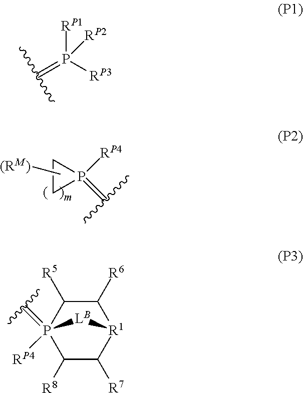

wherein

R^{P1} and R^{P2} are each independently selected from methyl, ethyl, isopropyl and phenyl;

R^{P3} is selected from the group consisting of
methyl and ethyl,
isopropyl,
cyclopentyl, t-butyl,
phenyl
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and -(CH₂)₂Q;

wherein Q is a C₅₋₆ heteroaryl group, optionally substituted with one or more groups R^{PA};

R^{P4} is selected from methyl and ethyl;

m is an integer selected from 1, 2 or 3;

R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;

—L^{B}— is methylene, ethylene or is absent;

when —L^{B}— is present, R^{P4} is absent and R¹ is selected from N, CH and CR^{PC};

when —L^{B}— is absent, R¹ is selected from the group consisting of
O,
NR^{Z},
SO₂,
CH₂, CHF, CF₂ and CHR^{PC};

wherein R^{Z} is selected from the group consisting of
—H, —C₁₋₃alkyl, —COC₁₋₃alkyl and —SO₂C₁₋₃alkyl;

R⁵ and R⁸ are each independently selected from —H and —R^{PC};

R⁶ and R⁷ are each independently selected from —H and —R^{PC};

wherein R^{PC} is selected from the group consisting of
C₁₋₃alkyl, optionally substituted with one or more groups R^{PD};

wherein R^{PA} is selected from the group consisting of
linear or branched C₁₋₃alkyl, C₂₋₆alkenyl or C₂₋₆alkynyl optionally substituted with one or more groups R^{AL},
—F, —Cl, —Br, —CN
—OH, —OR^{PE},
—CF₃, —CF₂H,
—COR^{PE},
—CH₂OH, —CH₂OR^{PE},
—COOH, —COOR^{PE}, —CONH₂, —CONHR^{PE}, —CONR^{PE}₂,
—OCOR^{PE}, —OCONH₂, —OCONHR^{PE}, —OCONR^{PE}₂,
—NH₂, —NHR^{PE}, —NR^{PE}₂,
—SO₂NH₂, —SO₂NHR^{PE}₂, —SO₂NR^{PE}₂,
—SO₂R^{PE},
—NHCOH, —NHCOR^{PE}, —NR^{PE}COH and —NR^{PE}COR^{PE};
   and R^{PB} is selected from the group consisting of
linear or branched C₁₋₃alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

R^{PE} is selected from
linear or branched C₁₋₄alkyl optionally substituted with one or more groups R^{PD};

and R^{PD} is selected from the group consisting of
F,
OH and OC₁₋₃alkyl

—L^{A}— is selected from
methylene optionally substituted with one or two groups R^{1A1},
ethylene optionally substituted with one or more groups R^{1A1}, and
a single bond;

R^{A} is selected from the group consisting of
(i) 5-membered heteroaromatic groups containing at least one heteroatom selected from N, O and S optionally C-substituted with one or more groups R^{A1}, and optionally N-substituted with one or more groups R^{NA1} with the proviso that when P^{X} is PMe₃ and L^{A} is a single bond, R^{A} is not selected from the groups (X1a) to (X1d)

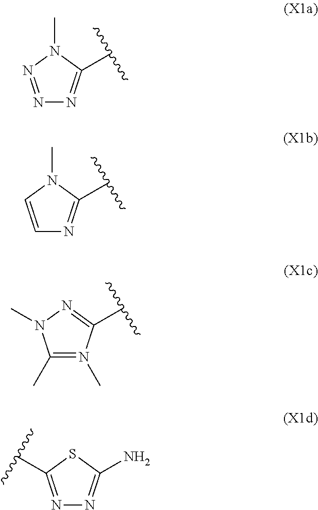

(ii) 6-membered aromatic groups or heteroaromatic groups containing 1 to 3 N atoms, substituted with one or more groups R^{A1}, with the proviso that when P^{X} is PMe₃ and L^{A} is a single bond, R^{A} is not selected from the groups (X2a) to (X2d)

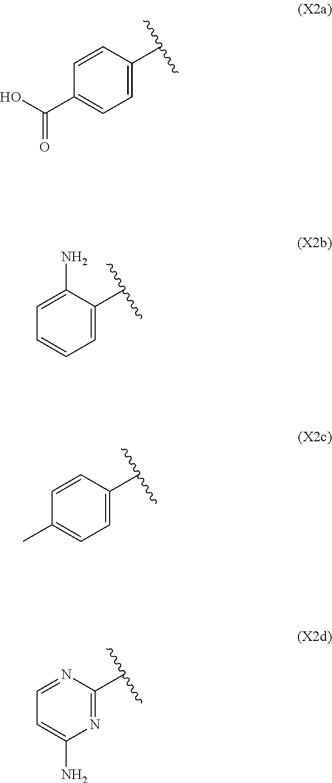

(iii) 8- to 10-membered bicyclyl or heterobicyclyl groups with the proviso that R^{A} is not selected from the group (A3) or the groups (X3a) to (X3b)

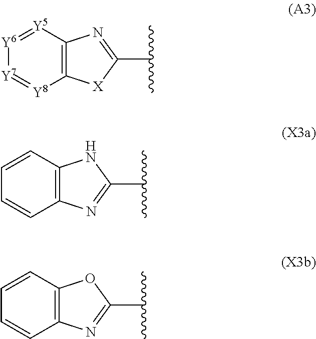

wherein one of Y⁵, Y⁶, Y⁷ and Y⁸ is selected from CH and N, and the others are
CH; and X is independently selected from NH, S and O; and
(iv) the groups (C1) to (C6)

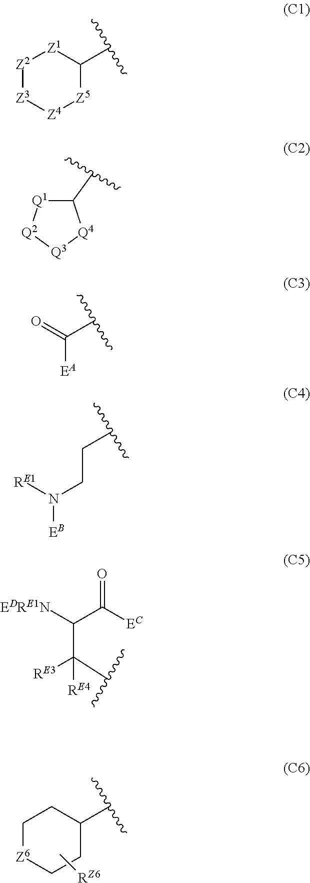

with the proviso that R^{A} is not the group (C3) when L is a single bond;

Z³ is selected from the group consisting of CH₂, CHR^{AL} and CR^{AL}₂;

one of Z¹, Z², Z⁴ and Z⁵ is selected from the group consisting of
CH₂, CHR^{AL}, CR^{AL}₂,
O,
NH, NR^{A2},
N(CO—R^{A2}), N(CO—NHR^{A2}), N(SO₂—R^{A2}) and N(CO₂—R^{A4});

the remainder of Z¹, Z², Z⁴ and Z⁵ are independently selected from the group consisting of
CH₂, CHR^{AL}, CR^{AL}₂, and
O;

with the provisos that the ring contains 0 or 1 oxygen atoms, that nitrogen atoms cannot be in a 1,2 or 1,3 relationship to each other, and that when Z¹ or Z⁵ is N, L cannot be a single bond;

one of Q¹ to Q⁴ is selected from the group consisting of
O,
NH, NR^{A2},
CH₂, CHR^{AL} and CR^{AL}₂,
N—CO—R^{A2}, N—CO—NHR^{A2}, N—SO₂—R^{A2} and N—CO₂—R^{A4}

the remainder of Q¹ to Q⁴ are independently selected from the group consisting of
NH, NR^{A2},
CH₂, CHR^{AL} and CR^{AL}₂;

with the proviso that the ring contains 0 or 1 oxygen atoms, that the ring contains 0 or 1 nitrogen atoms, and that when Q¹ or Q⁴ is N, L cannot be a single bond;

E^{A} is selected from the group consisting of
—O—R^{A2},
—NH—R^{A2},
—NR^{A2}₂,
—NR^{EA1}-E^{A1}-COR^{EA2} and —NR^{EA1}-E^{A2}-E^{A3}-COR^{EA2},

wherein E^{A1}, E^{A2} and E^{A3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EA1}— and —COR^{EA2} groups represent terminals of the alpha or pendent functionality of the amino acids respectively;

wherein the amino acid residues Asp and Glu may form amide bonds from either the alpha or pendent carboxylic acid functionality;

when E^{A1} is Pro, R^{EA1} is absent, otherwise R^{EA1} is R^{E1};

when E^{A2} is Pro, R^{EA1} is absent, otherwise R^{EA1} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{A2} and E^{A3} are present and E^{A3} is not Pro the nitrogen of the amide bond between E^{A2} and E^{A3} may be optionally substituted with R^{E1};

R^{EA2} is selected from —OR^{E7}, —NH₂, —NHR^{A2} and —NR^{A2}R^{E1};

R^{E1} is selected from H and linear or branched C₁₋₃alkyl;

E^{B} is selected from
E^{BA}, —CO-E^{B1}-NR^{EA}R^{E2} and —CO-E^{B2}-E^{B3}-NR^{EB}R^{E2},

wherein E^{B1}, E^{B2} and E^{B3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —CO—, —NR^{EA}R^{E2} and —NR^{EB}R^{E2} groups represent terminals of the alpha or pendent functionality of the amino acids;

wherein the amino acid residues Asp and Glu may form amide bonds from either the alpha or pendent carboxylic acid functionality

when E^{B1} is Pro, R^{EA} is absent, otherwise R^{EA} is R^{E1};

when E^{B3} is Pro, R^{EB} is absent, otherwise R^{EB} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{B2} and E^{B3} are present and E^{B2} is not Pro the nitrogen of the amide bond between E^{B2} and E^{B3} may be optionally substituted with R^{E1};

when E^{B} is E^{BA}, R^{E1} and E^{BA} together with the nitrogen atom to which they are attached form a group selected from
5- or 6-membered saturated heterocyclyl optionally substituted with one or more groups R^{AL}, and
5- or 6-membered heteroaryl optionally substituted with one or more groups R^{A1};

E^{C} is selected from
—OH,
—OR^{A2}
—NH₂, NHR^{A2}, NR^{A2}₂ and
—NR^{EC1}-E^{C1}-COR^{EC2}

wherein E^{C1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EC1}— and —COR^{EC2} groups represent terminals of the alpha or pendent functionality of the amino acids;

wherein the amino acid residues Asp and Glu may form amide bonds from either the alpha or pendent carboxylic acid functionality;

when E^{C1} is Pro, R^{EC1} is absent, otherwise R^{EC1} is R^{E1};

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH_{3;}

R^{EC2} is selected from —OR^{ES}, —NH₂, —NHR^{A2} and —NR^{A2}R^{E1};

R^{E3} and R^{E4} are independently selected from —H and —CH₃;

when R^{E1} is H and E^{C} is —OC₁₋₃alkyl, —NH₂ or —NHC₁₋₃alkyl, E^{D} is selected from
—H, and
—CO-E^{D1}-NR^{ED}—R^{E6}
   otherwise, E^{D} is selected from
R^{E5}, and
—CO-E^{D1}-NR^{ED}R^{E6};

wherein E^{D1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{ED}R^{E6}- and —CO— groups represent terminals of the alpha or pendent functionality of the amino acids;

wherein the amino acid residues Asp and Glu may form amide bonds from either the alpha or pendent carboxylic acid functionality;

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

when E^{D1} is Pro, R^{ED} is absent, otherwise R^{ED} is R^{E1};

with the proviso that R^{A} is not L-cysteine;

R_{E2}, R_{E5} and R^{E6} are independently selected from —H and —COCH₃;

R^{E7}, R^{E8} and R^{E9} are each independently selected from —H and —R^{A2};

Z⁶ is selected from N—CO—R^{A2}, N—CO—NHR^{A2}, N—SO₂—R^{A2};

R^{Z6} is one or two optional methyl substituents;

R^{A1} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2};

R^{A2} is selected from the group consisting of
linear or branched C₁₋₆alkyl, C₂₋₆alkenyl or C₂₋₆alkynyl optionally substituted with one or more groups R^{AT}, wherein the alkyl chain is optionally interrupted by one or more atoms selected from O and S;
OC₁₋₆alkyl;
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

where N is substituted by 2 R^{A2} groups, the N and the R^{A2} groups may together form a N-containing C₅₋₆ heterocycloalkyl group;

R^{NA1} is selected from linear or branched C₁₋₄alkyl;

R^{1A1} is selected from linear or branched unsubstituted C₁₋₃alkyl;

R^{A3} is selected from H and unbranched unsubstituted C₁₋₃alkyl;

R^{A4} is selected from linear or branched unsubstituted C₁₋₄alkyl;

R^{AL} is selected from the group consisting of
—F, —CN
—OH, —OR^{A2},
—CF₃, —CF₂H,
—COR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2}; and

wherein R^{AR} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{1A1},

In the fourth aspect, R^{A2} may be selected from the group consisting of
linear or branched C₁₋₆alkyl, C₂₋₆alkenyl or C₂₋₆alkynyl optionally substituted with one or more groups R^{AT};
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};

R^{P1} and R^{P2} may each be independently selected from methyl;

and R^{P3} may be selected from the group consisting of
methyl and ethyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q.

In some embodiments, where N is substituted by 2 R^{A2} groups, the N and the R^{A2} groups may together form a N-containing C₅₋₆ heterocycloalkyl group, optionally substituted with one or two groups selected from linear unsubstituted C₁₋₆ alkyl.

In some embodiments of the fourth aspect, when P^{X} is PMe₃ and L^{A} is a single bond, R^{A} is not selected from the group

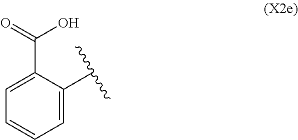 [SMILES: *c1ccccc1C(=O)O]

A fifth aspect of the present invention provides a pharmaceutical composition comprising a compound of the first or fourth aspects of the invention. The pharmaceutical composition may also comprise a pharmaceutically acceptable diluent or excipient. The fifth aspect of the present invention also provides the use of a compound of the first or fourth aspects of the invention in a method of therapy.

Another aspect of the invention provides a compound of formula VII′:

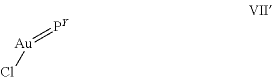

wherein

P^{Y} is independently selected from the group consisting of (P1), (P2) and (P3);

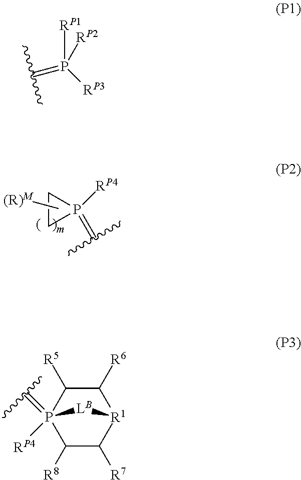

wherein

—L^{B}— is methylene, ethylene or is absent;

R^{P1} and R^{P2} are each independently selected from methyl;

R^{P3} is selected from the group consisting of
cyclopentyl, t-butyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;

wherein Q is a C₅₋₆ heteroaryl group, optionally substituted with one or more groups R^{PA};

R^{P4} is selected from methyl and ethyl;

m is an integer selected from 1, 2 or 3;

R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;

when —L^{B}— is present, R^{P4} is absent and R¹ is selected from N, CH and CR^{PC};

when —L^{B}— is absent, R¹ is selected from the group consisting of
O,
NR^{Z},
SO₂,
CH₂, CHF, CF₂ and CHR^{PC};

wherein R^{Z} is selected from the group consisting of
—H, —C₁₋₃alkyl, —COC₁₋₃alkyl and —SO₂C₁₋₃alkyl;

R⁵ and R⁸ are each independently selected from —H and —R^{PC};

R⁶ and R⁷ are each independently selected from —H and —R^{PC};

wherein R^{PC} is selected from the group consisting of
C₁₋₃alkyl, optionally substituted with one or more groups R^{PD};

and R^{PD} is selected from the group consisting of
F,
OH and OC₁₋₃alkyl.

In some embodiments, R^{P3} is selected from the group consisting of
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q.

Another aspect of the invention is a compound according to formula VII′ for use in the prevention or treatment of a bacterial infection. Another aspect is the use of a compound according to formula VII′ in the manufacture of a medicament for the prevention or treatment of a bacterial infection. Another aspect is a method of preventing or treating a bacterial infection in a human or animal, comprising administering to said patient an effective amount of a pharmaceutical composition containing a compound of formula VII′. Another aspect may relate to the treatment of fungal infection, e.g. by providing a compound of formula VII′ for use in the prevention or treatment of a fungal infection.

Another aspect of the invention provides a complex of formula VIII:

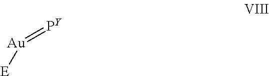

wherein

P^{Y} is independently selected from the group consisting of (P1), (P2) and (P3);

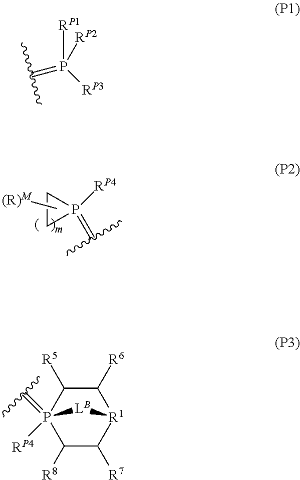

wherein

—L^{B}— is methylene, ethylene or is absent;

R^{P1} and R^{P2} are each independently selected from
methyl;

R^{P3} is selected from the group consisting of
cyclopentyl, t-butyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;

wherein Q is a C₅₋₆ heteroaryl group, optionally substituted with one or more groups R^{PA};

R^{P4} is selected from methyl and ethyl;

m is an integer selected from 1, 2 or 3;

R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;

when —L^{B}— is present, R^{P4} is absent and R¹ is selected from N, CH and CR^{PC};

when —L^{B}— is absent, R¹ is selected from the group consisting of
O,
NR^{Z},
SO₂,
CH₂, CHF, CF₂ and CHR^{PC};

wherein R^{Z} is selected from the group consisting of
—H, —C₁₋₃alkyl, —COC₁₋₃alkyl and —SO₂C₁₋₃alkyl;

R⁵ and R⁸ are each independently selected from —H and —R^{PC};

R⁶ and R⁷ are each independently selected from —H and —R^{PC};

wherein R^{PC} is selected from the group consisting of
C₁₋₃alkyl, optionally substituted with one or more groups R^{PD};
   and R^{PD} is selected from the group consisting of
F,
OH and OC₁₋₃alkyl;
   and

E is a residue of a thiol-containing or selenol-containing endogenous ligand or protein.

In some embodiments, R^{P3} is selected from the group consisting of
4-membered or 5-membered heterocycloalkyl group linked to phosphorus via a carbon atom in the ring, including a single heteroatom independently selected from NR^{Z}, O and S,
CF₃, —CH₂CF₃, —CH₂CF₂H, —CH₂CH₂OR^{PB},
CH₂Q and —(CH₂)₂Q.

Without wishing to be bound by theory, it is believed that compounds according to certain aspects of the invention, such as those according to formulae (I) or (II), may act as prodrugs which decompose within the body by cleavage of the Au—S bond and its replacement with a thiol-containing or selenol-containing endogenous ligand or protein, such as those entrained within the blood of an organism. The resultant complexes (i.e. complexes according to formula VIII) may then exert a therapeutic effect as described herein (see Crooke et al., Biochemical Pharmacology, 1986, Vol. 35, No. 20, 3423-3431 and Snyder et al., Biochemical Pharmacology, 1986, Vol. 35, No. 6, 923-932).

E is a “residue of a thiol-containing or selenol-containing endogenous ligand or protein”, in other words E is a ligand formed from the reaction of a thiol-containing or selenol-containing endogenous ligand or protein (E^{S}-SH or E^{SE}-SeH respectively) with the gold atom of the gold(I) phosphine (P^{Y}=Au) at a thiol or selenol group on the endogenous ligand or protein. As a result, -E has a structure selected from —S-E^{S} and —Se-E^{SE}, where E^{S} is the remainder of the thiol-containing endogenous ligand or protein (connected to Au via the S atom of a reacted thiol group) and E^{SE} is the remainder of the selenol-containing endogenous ligand or protein (connected to Au via the Se atom of a reacted selenol group).

It will be understood that the term “endogenous” indicates a ligand or protein originating within the body of a subject organism, such as within the body of a human subject.

Any ligand or protein containing an —SH or —SeH group may react with the gold(I) phosphine to provide a compound according to formula VIII. Examples of the groups -E are provided below.

In some embodiments, E is a residue of an endogenous low molecular weight thiol selected from cysteine (Cys), cysteinylglycine (CysGly) homocysteine (Hcy), and glutathione (GSH, L-γ-glutamyl-L-cysteinyl-glycine), N-acetylcysteine, thioglycolic acid, γ-glutamyl-cysteine, cysteinyl-glycine, lipoic acid and Coenzyme A.

In some embodiments, E is a residue of an endogenous low molecular weight selenol such as selenocysteine.

In some embodiments, E is a residue of an endogenous protein selected from human serum albumin, thioredoxin reductase (TrxR), glutathione peroxidase (GSPx), IkB kinase (IKK) complex, cathepsins and type I iodothyronine deiodinase.

In some cases, E may be a residue of an organism specific thiol-containing or selenol-containing endogenous ligand or protein such as mycothiol (present in Actinomycetes), bacillithiol (present in Firmicutes), γ-Glu-Cys (present in halobacteria and lactic acid bacteria), trypanothione (present in trypanosomes), ergothioneine (present in mycobacteria), coenzyme M or coenzyme B (present in methanogenic Archaea).

Another aspect of the invention is a compound according to formula VIII for use in the prevention or treatment of a bacterial infection. Another aspect is the use of a compound according to formula VIII in the manufacture of a medicament for the prevention or treatment of a bacterial infection. Another aspect is a method of preventing or treating a bacterial infection in a human or animal, comprising administering to said patient an effective amount of a pharmaceutical composition containing a compound of formula VIII. Another aspect may relate to the treatment of fungal infection, e.g. by providing a compound of formula VIII for use in the prevention or treatment of a fungal infection.

Further aspects of the invention relate generally to the use of the compounds of the present invention to inhibit microbial growth, sensitize the inhibition of microbial growth, inhibit biofilm formation or development, disrupt existing biofilms, reduce the biomass of a biofilm, and sensitize a biofilm and microorganisms within the biofilm to an antimicrobial agent.

In one aspect the invention relates to a method for inhibiting biofilm formation, comprising exposing a biofilm-forming microorganism to an effective amount of a compound of the invention. In some embodiments a compound of the invention is coated, impregnated or otherwise contacted with a surface or interface susceptible to biofilm formation. In some embodiments, the surface is a surface of a medical device such as: medical or surgical equipment, an implantable medical device or prosthesis (for example, venous catheters, drainage catheters (e.g. urinary catheters), stents, pacemakers, contact lenses, hearing-aids, percutaneous glucose sensors, dialysis equipment, drug-pump related delivery cannula, prostheses such as artificial joints, implants such as breast implants, heart valves, medical fixation devices such as rods, screws, pins, plates, or devices for wound repair such as sutures, and wound dressings such as bandages). In particular embodiments, the biofilm or biofilm-forming microorganism is on a bodily surface of a subject and exposure of the biofilm or biofilm-forming microorganism to a compound of the invention is by administration of the compound of the invention to the subject. In such instances, the biofilm or biofilm-forming microorganism may be associated with an infection, disease or disorder suffered by the subject or to which the subject is susceptible. In a related aspect of the invention, a medical device (such as those exemplified above) coated or impregnated with a compound of the invention is provided.

In another aspect the invention relates to a method for reducing the biomass of a biofilm and/or promoting the dispersal of microorganisms from a biofilm, comprising exposing the biofilm to an effective amount of a compound of the invention.

In yet another aspect the invention relates to a method for dispersing or removing, removing, or eliminating a biofilm, comprising exposing the biofilm to an effective amount of a compound of the invention. In some embodiments the biofilm is an existing, preformed or established biofilm.

In a further aspect the invention relates to a method for killing microorganisms within a biofilm, comprising exposing the biofilm to an effective amount of a compound of the invention. In some embodiments the biofilm is an existing, preformed or established biofilm.

In a yet further aspect the invention relates to a method of sensitizing a microorganism in a biofilm to an antimicrobial agent by exposing the biofilm to an effective amount of a compound of the invention. In some embodiments the antimicrobial agent is an antibiotic (e.g. rifampicin, gentamicin, erythromycin, lincomycin, linezolid or vancomycin) or an antifungal agent.

In one aspect the invention relates to a compound of the invention for use in a method of dispersing, removing or eliminating an existing biofilm, inhibiting biofilm formation, reducing the biomass of a biofilm, promoting the dispersal of microorganisms from a biofilm, killing microorganisms within a biofilm, sensitizing a microorganism in a biofilm to an antimicrobial agent, treating or preventing an infection, disease or disorder caused by a biofilm, inhibiting the growth of a microbial persister cell, killing a microbial persister cell, or treating or preventing an infection, disease or disorder caused by or associated with a microbial persister cell.

In another aspect the invention relates to a compound of the invention for use in a method of treating or preventing an infection, disease or disorder treatable by dispersing, removing or eliminating an existing biofilm, inhibiting biofilm formation, reducing the biomass of a biofilm, promoting the dispersal of microorganisms from a biofilm, killing microorganisms within a biofilm, sensitizing a microorganism in a biofilm to an antimicrobial agent, inhibiting the growth of a microbial persister cell, killing a microbial persister cell, or treating or preventing an infection, disease or disorder caused by or associated with a microbial persister cell.

In some aspects, the biofilm comprises bacteria, such as, for example, multi-drug resistant bacteria. In some aspects the bacteria are Gram positive bacteria. In some aspects the bacteria are Gram negative bacteria. In particular examples, the biofilm comprises, consists essentially of, or consists of S. aureus. In some aspects, the S. aureus is methicillin-resistant S. aureus (MRSA). In some embodiments, the biofilm comprises, consists essentially of, or consists of A. baumannii. In other embodiments, the biofilm comprises, consists essentially of, or consists of K. pneumoniae. In other embodiments, the biofilm comprises, consists essentially of, or consists of one or more of the bacteria listed in Table 1 herein. In further embodiments, the biofilms comprise bacterial species, including but not limited to, Staphylococcus spp., Streptococcus spp., Enterococcus spp., Listeria spp. and Clostridium spp., Klebsiella spp., Acinetobacter spp., Pseudomonas spp., Burkholderia spp., Erwinia spp., Haemophilus spp., Neisseria spp., Escherichia spp, Enterobacter spp., Vibrio spp. and/or Actinobacillus spp.

In some aspects, biofilm comprises lower eukaryotes, such as yeast, fungi, and filamentous fungi, including, but not limited to Candida spp., Pneumocystis spp., Coccidioides spp., Aspergillus spp., Zygomycetes spp., Blastoschizomyces spp., Saccharomyces spp., Malassezia spp., Trichosporon spp. and Cryptococcus spp. Example species include C. albicans, C. glabrata, C. parapsilosis, C. dubliniensis, C. krusei, C. tropicalis, A. fumigatus, and C. neoforms.

The biofilm may comprise one species of microorganism, or comprise two or more species of microorganism, i.e. be a mixed species biofilm. The mixed species biofilms may include two or more species of bacteria, two or more species of lower eukaryote (e.g. two or more fungal species, such as unicellular fungi, filamentous fungi and/or yeast), and/or both bacteria and lower eukaryotes, such as one or more species of bacteria and one or more species of lower eukaryotes. For example, the methods, uses and compositions provided herein are applicable to biofilms comprising one or more species of bacteria and one or more species of fungi, such as a yeast, unicellular fungi and/or filamentous fungi. The mixed species biofilm may thus comprise 2, 3, 4, 5, 10, 15, 20 or more species of microorganism, and the microorganisms within the biofilm may be bacteria and/or lower eukaryotes, such as unicellular fungi, filamentous fungi and/or yeast.

In one aspect the invention relates to a method for killing persister cells or inhibiting the growth of a microbial persister cell, comprising exposing the persister cell to an effective amount of a compound of the invention.

In another aspect the invention relates to a method for reducing the number, density or proportion of persister cells in a microbial population, comprising exposing the persister cell to an effective amount of a compound of the invention. In some embodiments the number, density or proportion of persister cells in a microbial population is reduced by at least 10% compared to an otherwise identical population not exposed to a compound of the invention; for example, at least 20%, at least 30%, at least 40%, at least 50%, at least 60%, at least 70%, at least 80%, at least 90%, at least 95%, at least 98%, at least 99%, at least 99.9%, or at least 99.99%.

In a further aspect the invention relates to a method of preventing the formation of microbial persister cells in a microbial population, the method comprising exposing the population to an effective amount of a compound of the invention.

In some aspects the persister cell is a bacterial or fungal persister cell. In some examples, the persister cell is a Gram negative bacterium. In some examples, the persister cell is a Gram positive bacterium. In some examples, the persister cell is a small colony variant. In particular embodiments, the persister cells are Staphylococcus spp. (including Staphylococcal SCVs), such as S. aureus (including methicillin resistant S. aureus (MRSA)), S. epidermidis, and S. capitis. In further embodiments, the persister cells are Pseudomonas spp. such as P. aeruginosa; Burkholderia spp. such as B. cepacia and B. pseudomallei; Salmonella serovars, including Salmonella Typhi; Vibrio spp. such as V. cholerae; Shigella spp.; Brucella spp. such as B. melitensis; Escherichia spp. such as E. coli; Lactobacillus spp. such as L. acidophilus; Serratia spp. such as S. marcescens; Neisseria spp. such as N. gonorrhoeae, or Candida spp., such as C. albicans.

The compounds of the invention can act together with other antimicrobial agents, allowing for increased efficacy of anti-microbial action. Accordingly, for any aspect described herein comprising exposing a biofilm, biofilm-forming microorganism, or a microbial persister cell to a compound of the invention, the present invention provides a corresponding further aspect comprising exposing the biofilm or biofilm-forming microorganism to a combination of compounds of the invention and at least one additional antimicrobial agent, such as, for example, an antibiotic or an anti-fungal agent. In particular examples, the antibiotic is selected from rifampicin, gentamicin, erythromycin, lincomycin and vancomycin.

The methods described herein may be performed, for example, in vivo, ex vivo, or in vitro.

### Definitions

Microbe/Microorganism: The terms “microbe/microorganism” as used herein pertain to bacteria and lower eukaryotes, such as fungi, including yeasts, unicellular fungi and filamentous fungi.

Antimicrobial agent: The term “antimicrobial agent” as used herein pertains to any agent that, alone or in combination with another agent, is capable of killing or inhibiting the growth of one or more species of microorganism. Antimicrobial agents include, but are not limited to, antibiotics, antifungals, detergents, surfactants, agents that induce oxidative stress, bacteriocins and antimicrobial enzymes (e.g. lipases, proteinases, pronases and lyases) and various other proteolytic enzymes and nucleases, peptides and phage. Reference to an antimicrobial agent includes reference to both natural and synthetic antimicrobial agents. Examples of antimicrobial agents include fluoroquinolones, aminoglycosides, glycopeptides, lincosamides, cephalosporins and related beta-lactams, macrolides, nitroimidazoles, penicillins, polymyxins, tetracyclines, and any combination thereof. For example, the methods of the present invention can employ acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin G benzathine, penicillin G potassium, penicillin G procaine, penicillin G sodium, penicillin V, penicillin V benzathine, penicillin V hydrabamine, and penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; bifonazolem; butoconazole; clotrimazole; econazole; fenticonazole; isoconazole; ketoconazole; miconazolel omoconazolel oxiconazolel sertaconazolel sulconazolel tioconazolel; albaconazole; fluconazole; isavuconazole; itraconazole; posaconazole; ravuconazole; terconazole; voriconazole;. abafungin; amorolfin; butenafine; naftifine; terbinafine; anidulafungin; caspofungin; and micafungin.

Biofilm: The term “biofilm” as used herein pertains to any three-dimensional, matrix-encased microbial community displaying multicellular characteristics. Accordingly, the term biofilm includes surface-associated biofilms as well as biofilms in suspension, such as flocs and granules. Biofilms may comprise a single microbial species or may be mixed species complexes, and may include bacteria as well as fungi, algae, protozoa, or other microorganisms.

Reducing the biomass of a biofilm: The term “reducing the biomass of a biofilm” is used herein to mean reducing the biomass of an area of a biofilm exposed to an effective amount of a compound of the invention as compared to the biofilm biomass of the area immediately before exposure to a compound of the invention. In some embodiments the “biomass” is the mass of cells present in the area of biofilm in addition to the extracellular polymeric substance (EPS) of the biofilm matrix. In some embodiments the “biomass” is only the mass of cells present in the area of biofilm (that is, the mass of the EPS is not counted as “biomass”). In some embodiments the biomass of the area of a biofilm exposed to an effective amount of a compound of the invention is at least 10% less than the biofilm biomass of the area immediately before exposure to a compound of the invention, the mass of the otherwise identical area of a biofilm which has not been exposed to a compound of the invention, for example, at least 20%, at least 30%, at least 40%, at least 50%, at least 60%, at least 70%, at least 80%, at least 90%, at least 95%, at least 98%, or at least 99% less than the biofilm biomass of the area immediately before exposure to a compound of the invention. In some embodiments the area of biofilm compared is 10⁻⁶ m²; in other embodiments the area of biofilm compared is 10⁻⁵ m², 10⁻⁴ m², or 10⁻³ m². In some embodiments a biofilm whose biomass has been reduced by at least 95% is deemed to have been “eliminated”, “dispersed” or “removed”. In some embodiments a biofilm whose biomass has been reduced by at least 99% is deemed to have been “eliminated”, “dispersed” or “removed”. In some embodiments a biofilm whose biomass has been reduced by at least 99.9% is deemed to have been “eliminated”, “dispersed” or “removed”. In some embodiments the change in biofilm biomass is assessed by a method comprising the steps of: i) washing the area of biofilm to remove non-adherent (planktonic) microorganisms, ii) assessing the area of biofilm biomass (i.e. the biomass “immediately before exposure to a compound of the invention”), iii) exposing the area of biofilm (or an otherwise identical area) to an effective amount of a compound of the invention for a period of time (for example, 24 hours), iv) washing the biofilm to remove non-adherent (planktonic) microorganisms, and v) assessing the area of biofilm biomass to obtain the ‘post-exposure’ biomass.

Promoting the dispersal of microorganisms from a biofilm: The term “promoting the dispersal of microorganisms from a biofilm” is used herein to mean reducing the number of microorganisms present in an area of a biofilm exposed to an effective amount of a compound of the invention as compared to the number of microorganisms present in the area immediately before exposure to a compound of the invention. In some embodiments the number of microorganisms in the area of a biofilm exposed to an effective amount of a compound of the invention is at least 10% less than the number of microorganisms present in the area immediately before exposure to a compound of the invention, for example, at least 20%, at least 30%, at least 40%, at least 50%, at least 60%, at least 70%, at least 80%, at least 90%, at least 95%, at least 98%, or at least 99% less than the number of microorganisms present in the area immediately before exposure to a compound of the invention. In some embodiments the change in number of microorganisms in an area of biofilm is assessed by a method comprising the steps of: i) washing the biofilm to remove non-adherent (planktonic) microorganisms, ii) counting the remaining microorganisms to obtain a ‘pre-exposure’ microorganism count (i.e. the count “immediately before exposure to a compound of the invention”), iii) exposing the biofilm to an effective amount of a compound of the invention for a period of time (for example, 24 hours), iv) washing the biofilm to remove non-adherent (planktonic) microorganisms, and v) counting the remaining microorganisms to obtain the ‘post-exposure’ microorganism count. In some embodiments a biofilm where number of microorganisms in an area has been reduced by at least 95% is deemed to have been “eliminated”, “dispersed” or “removed”. In some embodiments a biofilm where number of microorganisms in an area has been reduced by at least 99% is deemed to have been “eliminated”, “dispersed” or “removed”. In some embodiments a biofilm where number of microorganisms in an area has been reduced by at least 99.9% is deemed to have been “eliminated”, “dispersed” or “removed”.

Killing microorganisms within a biofilm: The term “killing microorganisms within a biofilm” is used herein to mean reducing the number of live microorganisms present in an area of a biofilm exposed to an effective amount of a compound of the invention as compared to the number of live microorganisms present in the area immediately before exposure to a compound of the invention. In some embodiments the biofilm is an existing, preformed or established biofilm. In some embodiments the number of live microorganisms in the area of a biofilm exposed to an effective amount of a compound of the invention is at least 10% less than the number of live microorganisms present in the area immediately before exposure to a compound of the invention, for example, at least 20%, at least 30%, at least 40%, at least 50%, at least 60%, at least 70%, at least 80%, at least 90%, at least 95%, at least 98%, or at least 99% less than the number of live microorganisms present in the area immediately before exposure to a compound of the invention. In some embodiments the change in number of microorganisms in an area of biofilm is assessed by a method comprising the steps of: i) washing the area biofilm to remove non-adherent (planktonic) microorganisms, ii) manually disperse the biofilm into solution (using, for example, scraping, sonication, and vortexing), iii) prepare serial dilutions, plate, and culture to estimate the number of colony forming unit (cfu) in the area of biofilm, iv) provide an otherwise identical area of biofilm and expose it to an effective amount of a compound of the invention for a period of time (for example, 24 hours), v) manually disperse the biofilm and estimate cfu as described above to obtain the ‘post-exposure’ microorganism count. The viability of the biofilm can be also assessed by allowing the biofilm to re-grow in compound free medium and assessing planktonic growth.

Dispersal: The term “dispersal” as used herein pertains to any to a biofilm and microorganisms making up a biofilm means the process of detachment and separation of cells and a return to a planktonic phenotype or behaviour of the dispersing cells.

Exposing: The term “exposing” as used herein means generally bringing into contact with. Exposure of a biofilm or biofilm-forming microorganism to an agent (e.g. a compound of the invention) includes administration of the agent to a subject harbouring the microorganism or biofilm, or otherwise bringing the microorganism or biofilm into contact with the agent itself, such as by contacting a surface on which the biofilm or biofilm-forming microorganism are present with the agent. In some embodiments, the biofilm or biofilm-forming microorganisms are exposed to a compound of the invention by coating, impregnating or otherwise contacting a surface or interface susceptible to biofilm formation to an effective amount of the compound. Surfaces that may be exposed, coated, or impregnated with a compound of the invention include those present in a range of industrial and domestic settings, including but not limited to, domestic, medical or industrial settings (e.g. medical and surgical devices, and surfaces within hospitals, processing plants and manufacturing plants), as well as internal and external surfaces of the body of a subject. In the present disclosure the terms “exposing”, “administering” and “contacting” and variations thereof may, in some contexts, be used interchangeably.

Inhibiting: The term “inhibiting” and variations thereof such as “inhibition” and “inhibits” as used herein in relation to microbial growth refers to any microbiocidal or microbiostatic activity of an agent (e.g. a compound of the invention) or composition. Such inhibition may be in magnitude and/or be temporal or spatial in nature. Inhibition of the growth of a microorganism by an agent can be assessed by measuring growth of the microorganism in the presence and absence of the agent. The growth can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the growth of the same microorganism that is not exposed to the agent.

The term “inhibiting” and variations thereof such as “inhibition” and “inhibits” as used herein in relation to biofilms means complete or partial inhibition of biofilm formation and/or development and also includes within its scope the reversal of biofilm development or processes associated with biofilm formation and/or development. Further, inhibition may be permanent or temporary. The inhibition may be to an extent (in magnitude and/or spatially), and/or for a time, sufficient to produce the desired effect. Inhibition may be prevention, retardation, reduction or otherwise hindrance of biofilm formation or development. Such inhibition may be in magnitude and/or be temporal or spatial in nature. Inhibition of the formation or development of a biofilm by a compound of the invention can be assessed by measuring biofilm mass or microbial growth in the presence and absence of a compound of the invention. The formation or development of a biofilm can be inhibited by a compound of the invention by at least about 10%, 15%, 20%, 25%, 30%, 35%, 40%, 45%, 50%, 55%, 60%, 65%, 70%, 75%, 80%, 85%, 90%, 95% or more compared to the formation or development of a biofilm that is not exposed to a compound of the invention.

Sensitize: The terms “sensitize” or “sensitizing” as used herein mean making a biofilm or microorganisms within a biofilm more susceptible to an antimicrobial agent. The sensitizing effect of a compound of the invention, on a biofilm or microorganisms within the biofilm can be measured as the difference in the susceptibility of the biofilm or microorganisms (as measured by, for example, microbial growth or biomass of the biofilm) to a second antimicrobial agent with and without administration of the compound. The sensitivity of a sensitized biofilm or microorganism (i.e. for example, a biofilm or microorganism exposed to an agent such as a compound of the invention) to a antimicrobial agent can be increased by at least about 10%, 20%, 30%, 40%, 50%, 60%, 70%, 80%, 90%, 100%, 150%, 200%, 250%, 300%, 350%, 400%, 450%, 500% or more compared to the sensitivity of an unsensitized biofilm or microorganism (i.e. a biofilm or microorganism not exposed to the agent). In some embodiments sensitizing effect of a compound of the invention on a biofilm or microorganisms within the biofilm can be measured by the difference in Minimum Inhibitory Concentration (MIC) of a second antimicrobial administered either in combination with a compound of the invention, or alone. For example, in some embodiments the MIC of a combination of a compound of the invention and the second antimicrobial is at least 10% lower than the MIC of the second antimicrobial administered alone; such as at least 20% lower, at least 30% lower, at least 40% lower, at least 50% lower, at least 60% lower, at least 70% lower, at least 80% lower, at least 90% lower, at least 95% lower, at least 99% lower, or at least 99.9% lower than the MIC of the second antimicrobial administered alone. The sensitization of a microorganism may also occur outside of a bioflim.

Surface: The term “surface” as used herein includes both biological surfaces and non-biological surfaces. Biological surfaces typically include surfaces both internal (such as organs, tissues, cells, bones and membranes) and external (such as skin, hair, epidermal appendages, seeds, plant foliage) to an organism. Biological surfaces also include other natural surfaces such as wood or fibre. A non-biological surface may be any artificial surface of any composition that supports the establishment and development of a biofilm. Such surfaces may be present in industrial plants and equipment, and include medical and surgical equipment and medical devices, both implantable and non-implantable. Further, for the purposes of the present disclosure, a surface may be porous (such as a membrane) or non-porous, and may be rigid or flexible.

Infection, disease or disorder caused by a biofilm/infection, disease or disorder caused by or associated with a microbial persister cell: The term “Infection, disease or disorder caused by a biofilm” as used herein is used to describe conditions, diseases and disorders associated with, characterised by, or caused by biofilms and biofilm-forming microorganisms. Similarly, the term “Infection, disease or disorder caused by or associated with a microbial persister cell” as used herein is used to describe conditions, diseases and disorders associated with, characterised by, or caused by microbial persister cells. For example, a variety of microbial infections are known to be associated with biofilm formation and/or persister cells, such as cellulitis, impetigo, mastitis, otitis media, bacterial endocarditis, sepsis, toxic shock syndrome, urinary tract infections, pulmonary infections (including pulmonary infection in patients with cystic fibrosis), pneumonia, dental plaque, dental caries, periodontitis, bacterial prostatitis and infections associated with surgical procedures or burns. For example, S. aureus and S. epidermidis cause or are associated with cellulitis, impetigo, mastitis, otitis media, bacterial endocarditis, sepsis, toxic shock syndrome, urinary tract infections, pulmonary infections (including pulmonary infection in patients with cystic fibrosis), pneumonia, dental plaque, dental caries and infections associated with surgical procedures or burns. In other examples, K. pneumoniae can cause or be associated with pneumonia, sepsis, community-acquired pyogenic liver abscess (PLA), urinary tract infection, and infections associated with surgical procedures or burns. In further examples, A. baumannii can cause or be associated with bacteremia, pneumonia, meningitis, urinary tract infection, and infections associated with wounds. In still further examples, P. aeruginosa can cause or be associated with respiratory tract infections (including pneumonia), skin infections, urinary tract infections, bacteremia, infection of the ear (including otitis media, otitis externa and otitis interna), endocarditis and bone and joint infections such as osteomyelitis. Candida spp. such as C. albicans, Cryptococcus spp. such as C. neoformans, as well as other fungi such as Trichosporon spp., Malassezia spp., Blastoschizomyces spp., Coccidioides spp. and Saccharomyces spp. (e.g. S. cerevisiae) may cause or be associated with infections related to the implantation or use of medical or surgical devices, such as catheterization or implantation of heart valves.

Persister cell(s): The term “persister cell(s)” as used herein pertains to metabolic variants of wild type microbial cells that are phenotypically characterized by their slow growth rate, which is typically 30%, 25%, 20%, 15%, 10%, 5% or less of the growth rate of the wild-type counterpart. In some embodiments, the persister cells are dormant and have, for example, no detectable cell division in a 24 hour period. Further, persister cells typically form colonies that are approximately 30%, 25%, 20%, 15%, 10%, 5% or less of the size of the colonies formed by their wild-type counterparts. Reference to persister cells includes reference to persister cells of any microbial genera or species, including, but not limited to, bacterial and lower eukaryotic, such as fungal, including yeast, persister cells. In some examples, the persister cell is a Gram negative bacterium. In some examples, the persister cell is a Gram positive bacterium. Exemplary persister cells include, but are not limited to, those of Staphylococcus spp., such as S. aureus, S. epidermidis, and S. capitis; Pseudomonas spp. such as P. aeruginosa; Burkholderia spp. such as B. cepacia and B. pseudomallei; Salmonella serovars, including Salmonella Typhi; Vibrio spp. such as V. cholerae; Shigella spp.; Brucella spp. such as B. melitensis; Escherichia spp. such as E. coli; Lactobacillus spp. such as L. acidophilus; Serratia spp. such as S. marcescens; Neisseria spp. such as N. gonorrhoeae, as well as Candida spp., such as C. albicans.

C₁₋₆ alkyl: The term “C₁₋₆ alkyl” as used herein, pertains to a monovalent moiety obtained by removing a hydrogen atom from a carbon atom of a saturated hydrocarbon compound having from 1 to 6 carbon atoms.

Examples of saturated alkyl groups include, but are not limited to, methyl (C₁), ethyl (C₂), propyl (C₃), butyl (C₄), pentyl (C₅) and hexyl (C₆).

Examples of saturated linear alkyl groups include, but are not limited to, me1thyl (C₁), ethyl (C₂), n-propyl (C₃), n-butyl (C₄), n-pentyl (C₅) and n-hexyl (C₆).

Examples of saturated branched alkyl groups include iso-propyl (C₃), iso-butyl (C₄), sec-butyl (C₄), tert-butyl (C₄), iso-pentyl (C₅), neopentyl (C₅), iso-hexyl (C₆) and neohexyl (C₆).

C₂₋₆ alkenyl: The term “C₂₋₆ alkenyl” as used herein, pertains to a C₂₋₆ alkyl group having one or more carbon-carbon double bonds. Examples of unsaturated alkenyl groups include, but are not limited to, ethenyl (vinyl, —CH═CH₂), 1-propenyl (−CH═CH—CH₃), 2-propenyl (allyl, —CH—CH═CH₂) and isopropenyl (1-methylvinyl, —C(CH₃)═CH₂).

C₂₋₆ alkynyl: The term “C₂₋₆ alkynyl” as used herein, pertains to a C₂₋₆ alkyl group having one or more carbon-carbon triple bonds. Examples of unsaturated alkynyl groups include, but are not limited to, ethynyl (—C≡CH) and 2-propynyl (propargyl, —CH₂—C≡CH).

C₃₋₆ cycloalkyl: the term “C₃₋₆ cycloalkyl” as used herein, pertains to a monovalent moiety obtained by removing a hydrogen atom from a carbon atom of a saturated cyclic core having 3, 4, 5 or 6 atom in the cyclic core all of which are carbon atoms. Examples of C₃₋₆ cycloalkyl include, but are not limited to, cyclopropyl, cyclohexyl and cyclopentyl.

C₅₋₆ cycloalkenyl: The term “C₅₋₆ cycloalkenyl” as used herein, pertains to a C₃₋₆cycloalkyl group having one or more carbon-carbon double bonds.

C₄₋₆ heterocycloalkyl: The term “C₄₋₆ heterocycloalkyl” as used herein, pertains to a monovalent moiety obtained by removing a hydrogen atom from a ring atom of a heterocyclic compound, which moiety has from 4 to 6 ring atoms, of which from 1 to 3 are ring heteroatoms selected from O, S and N. In this context, the prefixes denote the number of ring atoms, or range of number of ring atoms, whether carbon atoms or heteroatoms

Examples of monocyclic heterocycloalkyl groups include, but are not limited to, those derived from:

N₁: azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆);

C₁: oxetane (C₄), oxolane (tetrahydrofuran) (C₅), oxole (dihydrofuran) (C₅), oxane (tetrahydropyran) (C₆), dihydropyran (C₆), pyran (C₆);

S₁: thietane (C₄), thiolane (tetrahydrothiophene) (C₅), thiane (tetrahydrothiopyran) (C₆);

O₂: dioxolane (C₅), dioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

C₅₋₆ heterocycloalkenyl: The term “C₅₋₆ heterocycloalkenyl” as used herein, pertains to a C₅₋₆ heterocycloalkyl group having one or more carbon-carbon or carbon-nitrogen double bonds.

Heterobicyclyl: The term “heterobicyclyl” as used herein, pertains to a bicyclic ring, wherein 1, 2, or 3 ring carbons are replaced with a heteroatom selected from the group consisting of O, S and N. In some embodiments, one of the rings is aromatic. The bicylic rings may be spiro or fused. Examples of a heterobicyclic group include, but are not limited to, 2,5-diaza-bicyclo[2.2.1]hept-2-yl, 7-aza-bicyclo[2.2.1]hept-7-yl, 1,3-dihydro-isoindolyl, 3,4-dihydro-1H-isoquinolinyl, octahydro-cyclopenta[c]pyrrolyl and the like

C₅₋₆ heteroaryl: the term C₅₋₆ heteroaryl as used herein, pertains to a monovalent moiety obtained by removing a hydrogen atom from a ring atom of an aromatic structure having between one and three atoms that are not carbon forming part of said ring. Wherein, those atoms that are not carbon can be chosen independently from the list nitrogen, oxygen and sulphur.

Examples of C₅₋₆ heteroaryl groups include, but are not limited to, groups derived from:

N₁: pyridine (C₆);

N₁O₁: oxazole (C₅), isoxazole (C₅);

N₂O₁: oxadiazole (furazan) (C₅);

N₂S₁: thiadiazole (C₅)

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅).

### Further Embodiments

In some embodiments, P^{X} or P^{Y} is P1.

In some embodiments, R^{P1} is methyl. In other embodiments, R^{P1} is ethyl.

In some embodiments, R^{P2} is methyl. In other embodiments, R^{P2} is ethyl.

In some embodiments, both R^{P2} and R^{P2} are methyl. In other embodiments, both R^{P1} and

R^{P2} are ethyl. In further embodiments, R^{P1} is methyl and R^{P2} is ethyl.

In some embodiments, R^{P1} is isopropyl. In some embodiments, R^{P1} is phenyl. In some embodiments, both R^{P1} and R^{P2} are isopropyl. In some embodiments, both R^{P1} and R^{P2} are phenyl.

In some embodiments, R^{P1} is methyl, R^{P2} is phenyl and R^{P3} is selected from methyl and phenyl.

In some embodiments, R^{P3} is methyl. In other embodiments, R^{P3} is ethyl.

In some embodiments, R^{P3} is isopropyl. In some embodiments, R^{P3} is t-butyl. In some embodiments, R^{P3} is cyclopentyl. In some embodiments, R^{P3} is phenyl.

In some embodiments, P^{X} is PMe₃.

In some embodiments, P^{X} is PEt₃.

In some embodiments, P^{X} is PEt₂Me.

In some embodiments, P^{X} is PEtMe₂.

In some embodiments, P^{X} is PMe₃.

In some embodiments, P^{X} is P(Ph)₃.

In some embodiments, P^{X} is P(i-Pr)₃.

In some embodiments, P^{X} is P(Me)(Ph)₂.

In some embodiments, P^{X} is P(Ph)(Me)₂.

In some embodiments, R^{P3} is a 4-membered or 5-membered heterocycloalkyl group linked to phosphorus via a carbon atom in the ring, including a single heteroatom independently selected from N, O and S. In these embodiments, R^{P3} may be selected from azetidinyl, oxetanyl, thietanyl, pyrrolidinyl, tetrahydrofuranyl and thiolanyl. In some of these embodiments, R^{P3} may be oxetanyl or tetrahydrofuranyl.

In some embodiments, P^{X} is:

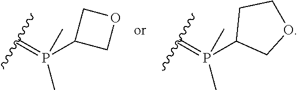

In some embodiments, R^{P3} is selected from the group consisting of —CF₃, —CH₂CF₃, —CH₂CF₂H and —CH₂CH₂OR^{PB}. In some of these embodiments, R^{PB} may be a linear or branched C₁₋₆ alkyl, e.g. methyl.

In some embodiments, P^{X} or P^{Y} is selected from:

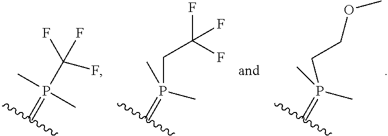

In some embodiments, R^{P3} is selected from the group consisting of —CH₂Q and —(CH₂)₂Q. In some of these embodiments, R^{P3} is —CH₂Q. In other of these embodiments, R^{P3} is —(CH₂)₂Q.

In any of these embodiments, Q is a C₅₋₆ heteroaryl group, optionally substituted with one or more groups R^{PA}. In some of these embodiments, Q may be unsubstituted. In other of these embodiments, Q may be substituted, and in particular, if Q comprises a N ring atom, this may be substituted by a methyl group.

In some embodiments, Q is independently selected from:

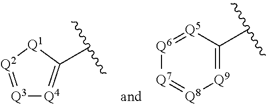

wherein

Q¹ is independently selected from O, S and NR^{PE};

each of Q² to Q⁴ is independently selected from N and CR^{PA};

two of Q⁵ to Q⁹ is selected from CR^{PA}, one other of Q⁵ to Q⁹ is selected from N and the remainder are selected from N, CH and CR^{PA}.

In some embodiments, P^{X} or P^{Y} is selected from:

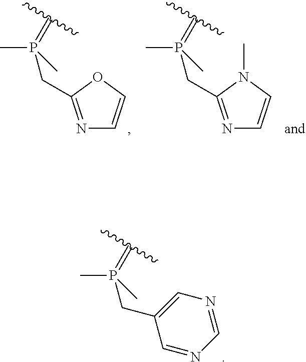

In some embodiments, R^{P1} and R^{P2} are methyl and R^{P3} is cyclopentyl. In some embodiments, R^{P1} and R^{P2} are methyl and R^{P3} is t-butyl.

In some embodiments, P^{X} or P^{Y} is P2.

In some embodiments, R^{P4} is methyl. In other embodiments, R^{P4} is ethyl.

In some embodiments, m is 1. In other embodiments, m is 2. In further embodiments, m is 3.

In some embodiments, the ring in P2 is not substituted. In other embodiments, there is one R^{M} substituent on the ring in P2. In further embodiments, there are two R^{M} substituents on the ring in P2.

In some embodiments, R^{M} is R^{PC} and R^{PC} may be methyl. In other embodiments, R^{M} is OH. In further embodiments, R^{PC} is OMe.

In some embodiments, P^{X} or P^{Y} is selected from:

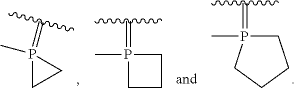

In some embodiments, P^{X} or P^{Y} is P3.

In some embodiments, —L^{B}— is methylene. In other embodiments, —L^{B}— is ethylene.

When —L^{B}— is present, R^{P4} is absent and R¹ is selected from N, CH and CR^{PC}. In some of these embodiments, R¹ is N. In other of these embodiments, R¹ is CH. In further of these embodiments, R¹ is CR^{PC}. In some embodiments, R^{PC} is unsubstituted C₁₋₃ alkyl, e.g. methyl.

When L^{B} is absent, in some embodiments R¹ is selected from the group consisting of O, NR^{Z}, and SO₂. In these embodiments, R^{Z} may be selected from H and C₁₋₃ alkyl e.g. methyl.

When L^{B} is absent, in some embodiments R¹ is selected from the group consisting of CH₂, CHF, CF₂ and CHR^{PC}. In some of these embodiments, R¹ is CH₂. In other of these embodiments, R¹ is CHF. In other of these embodiments, R¹ is CF₂. In further of these embodiments, R¹ is CHR^{PC}. In some embodiments, R^{PC} is unsubstituted C₁₋₃ alkyl, e.g. methyl.

In some embodiments, P^{X} or P^{Y} is selected from:

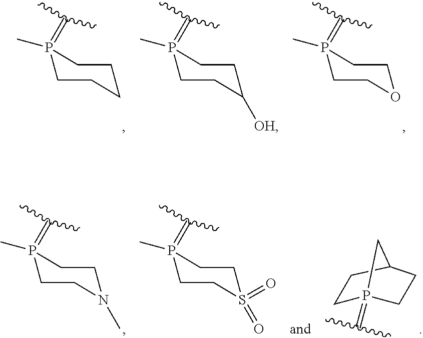

The First and Second Aspects

L^{C}

In some embodiments, —L^{C}— is absent.

In some embodiments, —L^{C}— is methylene.

In some embodiments, —L^{C}— is ethylene.

R^{B}

In some embodiments, R^{B} is A1:

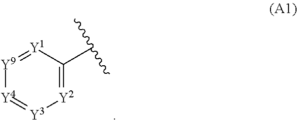

In some embodiments, one of Y¹, Y², Y³, Y⁴ and Y⁹ is N. In some of these embodiments, Y¹ is N and Y², Y³, Y⁴ and Y⁹ are CH. In others of these embodiments, Y³ is N and Y¹, Y², Y⁴ and Y⁹ are CH. In others of these embodiments, Y⁴ is N and Y¹, Y², Y³ and Y⁹ are CH.

In these embodiments, A1 is pyridyl.

In some embodiments, two of Y¹, Y², Y³, Y⁴ and Y⁹ are N. In some of these embodiments, Y¹, Y⁴ and Y⁹ are CH and Y² and Y³ are N. In others of these embodiments, Y², Y⁴ and Y⁹ are CH and Y¹ and Y³ are N. In others of these embodiments, Y³, Y⁴ and Y⁹ are CH and Y¹ and Y² are N. In some of these embodiments, Y¹ and Y⁴ are N and Y², Y³ and Y⁹ are CH. In others of these embodiments, Y² and Y⁴ is N and Y¹, Y³, and Y⁹ are CH. In others of these embodiments, Y³ and Y⁴ are N and Y¹, Y² and Y⁴ are CH. In others of these embodiments, Y³ and Y⁹ are N and Y¹, Y² and Y⁴ are CH. In these embodiments, A1 is selected from pyrimidinyl, pyridazinyl and pyrazinyl.

In some embodiments, all of Y¹, Y², Y³, Y⁴ and Y⁹ are CH, i.e. A1 is phenyl.

In some embodiments, R^{B} is A2:

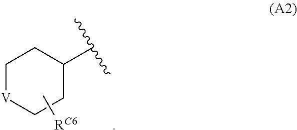

In some of these embodiments, V is O.

In other of these embodiments, V is CH—OR^{O1}, where R^{O1} is selected from H and C₁₋₃ unbranched alkyl. In some of these embodiments, R^{O1} is H. In others of these embodiments, R^{O1} is C₁₋₃ unbranched alkyl, e.g. methyl, ethyl, n-propyl.

In other of these embodiments, V is N—CO₂—R^{C2}, where R^{C2} is either C₁₋₃ unbranched alkyl or C₃₋₄ branched alkyl. In some of these embodiments, R^{C2} is C₁₋₃ unbranched alkyl, i.e. methyl, ethyl, n-propyl. In others of these embodiments, R^{C2} is C₃₋₄ branched alkyl, i.e. iso-propyl, iso-butyl, sec-butyl and tert-butyl.

In other of these embodiments, V is N—R^{N2}, where R^{N2} is C₁₋₃ unbranched alkyl, i.e. methyl, ethyl, n-propyl. In some embodiments, R^{N2} is methyl.

In some of these embodiment, there are no optional methyl substituents (represented by R^{C6}).

In other of these embodiments, there is a single methyl substituent represented by R^{C6}.

In other of these embodiments, there are two methyl substituents represented by R^{C6}.

In some embodiments, R^{B} is A3:

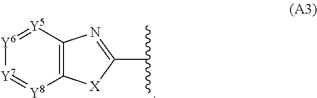

In some of these embodiments, X is NH. In others of these embodiments, X is O.

In some of these embodiments, all of Y⁵, Y⁶, Y⁷ and Y⁸ are CH. In others of these embodiments, one of Y⁵, Y⁶, Y⁷ and Y⁸ is N. In some of these embodiments, Y⁵ may be N. In some of these embodiments Y⁶ may be N. In some of these embodiments Y⁷ may be N. In some of these embodiments Y⁸ may be N.

In some embodiments, R^{B} is A4:

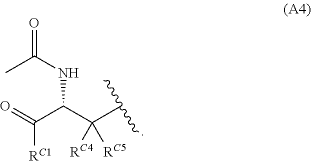

In some of these embodiments, R^{C1} is O13 R^{O2}. R^{O2} is C₁₋₃ unbranched alkyl, i.e. methyl, ethyl, n-propyl.

In others of these embodiments, R^{C1} is NHR^{N1}. In some of these embodiments, R^{N1} is H.

In others of these embodiments, R^{N1} is C₁₋₃ unbranched alkyl, i.e. methyl, ethyl, n-propyl.

In some of these embodiments, R^{C4} and R^{C5} are both H.

In other of these embodiments, R^{C4} is H and R^{C5} is Me.

In other of these embodiments, R^{C4} and R^{C5} are both Me.

In some embodiments, R^{B} is A5:

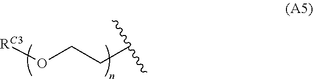

In some of these embodiments, R^{C3} is C₁₋₃ unbranched alkyl, i.e. methyl, ethyl, n-propyl.

In others of these embodiments R^{C3} is C₂H₄CO₂H.

In some of these embodiments n is an integer from 4 to 8. In some of these embodiments, n is 7 or 8.

The Third and Fourth Aspects

L^{A}

In some embodiments, L^{A} is methylene substituted with one or two groups R^{1A1}.

In some embodiments, L^{A} is methylene substituted with one or two methyl groups.

In some embodiments, L^{A} is methylene.

In some embodiments, L^{A} is ethylene substituted with one or more groups R^{1A1}.

In some embodiments, L^{A} is ethylene substituted with one or more methyl groups.

In some embodiments, L^{A} is ethylene.

In some embodiments, L^{A} is a single bond.

R^{A}

In some embodiments, R^{A} is a 5-membered heteroaromatic group containing up to 4 heteroatoms selected from N, O and S, at least one of which being N.

In some embodiments, R^{A} is a 5-membered heteroaromatic group containing up to 4 heteroatoms selected from N and O, at least one of which being N.

In some embodiments, R^{A} is a 5-membered heteroaromatic group connected to sulfur at a ring carbon and containing up to 4 heteroatoms selected from N, O and S, at least one of which being N.

In some embodiments, R^{A} is a 5-membered heteroaromatic group containing up to 4 heteroatoms selected from N.

In some embodiments, R^{A} is unsubstituted tetrazolyl.

In some embodiments, R^{A} is a 5-membered heteroaromatic group containing at least one heteroatom selected from N, O and S optionally N-substituted with one or more groups selected from
linear C₁₋₃alkyl;
   and optionally C-substituted with one or more groups selected from
linear or branched C₁₋₃alkyl optionally substituted with one or more groups R^{AL}.

In some embodiments, R^{A} is a 5-membered heteroaromatic group containing at least one heteroatom selected from N, O and S optionally N-substituted with one or more groups selected from
methyl and ethyl
   and optionally C-substituted with one or more groups selected from
linear or branched C₁₋₃alkyl.

In some embodiments, R^{A} is a 5-membered heteroaromatic group containing at least one heteroatom selected from N, O and S optionally N-substituted with one or more methyl groups, and optionally C-substituted with one or more methyl groups.

In some embodiments, P^{X} is P(CH₃)₃ and R^{A} is a 5-membered heteroaromatic group containing a single heteroatom selected from N, O and S.

In some embodiments, P^{X} is P(CH3)3 and R^{A} is a 5-membered heteroaromatic group selected from the group consisting of
unsubstituted tetrazolyl;
unsubstituted pyrazolyl or imidazolyl;
oxazolyl or isoxazolyl, optionally C-substituted with one or more groups R^{A1}, and optionally N-substituted with one or more groups R^{NA1}; and
triazolyl, optionally mono- or di-substituted with one or two groups selected from linear or branched C₁₋₃alkyl.

In some embodiments, R^{A} is selected from

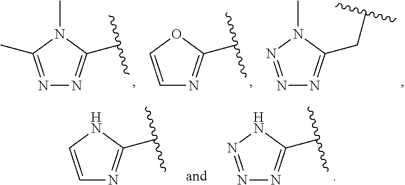

In some embodiments, R^{A} is

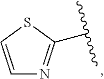 [SMILES: *c1nccs1]

In some embodiments, R^{A} is selected from 6-membered aromatic carbocyclic groups substituted with one or more groups selected from
linear or branched C₁₋₆alkyl, optionally substituted with one or more groups R^{AL},
—F, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2, —NRA}₂COH and —NR^{A2}COR^{A2}.

In some embodiments, R^{A} is selected from 6-membered aromatic carbocyclic groups substituted with one or more groups selected from
linear or branched C₁₋₆alkyl, optionally substituted with one or more groups R^{AL},
—F, —CN
—OH, —OC₁₋₃alkyl,
—CF₃, —CF₂H,
—COC₁₋₃alkyl,
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—SO₂NH₂, —SO₂NHR^{A2}₂, —SO₂NR^{A2}₂, and
—SO₂R^{A2}.

In some embodiments, R^{A} is selected from 6-membered aromatic carbocyclic groups substituted with one or more groups selected from
linear or branched C₁₋₃alkyl, optionally substituted with one or more groups R^{AL},
—F, —CN
—OH, —OC₁₋₃alkyl,
—CF₃,
—COOH, —CONH₂, —CONHR^{A2}, —CONR^{A2}₂,
—OCOR^{A2},
—SO₂NH₂, —SO₂NHR^{A2}₂, —SO₂NR^{A2}₂, and
—SO₂R^{A2}.

In some embodiments, R^{A} is selected from 6-membered aromatic carbocyclic groups substituted with one or more groups selected from
linear or branched C₁₋₃alkyl, optionally substituted with one or more groups R^{AL},
—F, —CN
—OH, —OC₁₋₃alkyl,
—CF₃,
—COOH, —CONH₂, —CONHR^{A2}, —CONR^{A2}₂,
—OCOR^{A2}, and
—SO₂C₁₋₃alkyl.

In some embodiments, R^{A} is selected from 6-membered aromatic carbocyclic groups substituted with one or more groups selected from
linear or branched C₁₋₃alkyl, optionally substituted with one or more groups R^{AL},
—F, —CN
—OH, —OMe,
—CF₃,
—COOH, —CONH₂,
—OMe, and
—SO₂Me.

In some embodiments, R^{A} is selected from 6-membered aromatic carbocyclic groups substituted with one or more groups selected from
linear or branchedC₁₋₆alkyl, optionally substituted with one or more groups R^{AL},
—F,
—OH, —OMe,
—CF₃ and
—COOH.

In some embodiments, R^{A} is selected from 6-membered aromatic carbocyclic groups ortho- and/or meta-substituted with one or more groups selected from
linear or branchedC₁₋₆alkyl, optionally substituted with one or more groups R^{AL},
—F, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2};
   and/or para-substituted with a group selected from
linear or branched C₁₋₆alkyl, optionally substituted with one or more groups R^{AL},
—F, —CN
—OH, —OR^{A2},
—CF₃, —CF₂H,
—COR^{A2},
—CH₂OH, —CH₂OR^{A2},
—COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NRA2₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2}.

In some embodiments, R^{A} is selected from

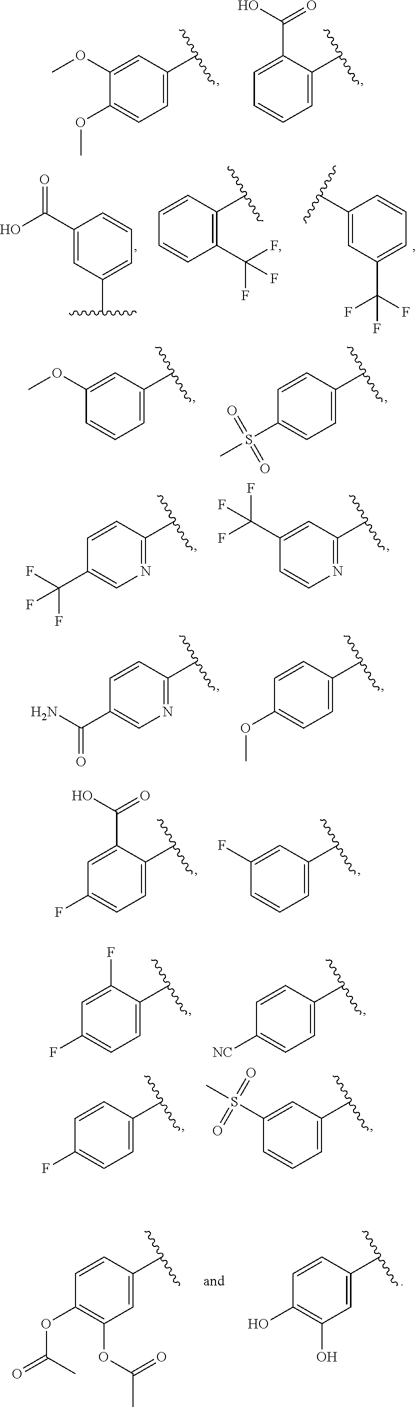

In some embodiments, R^{A} is selected from

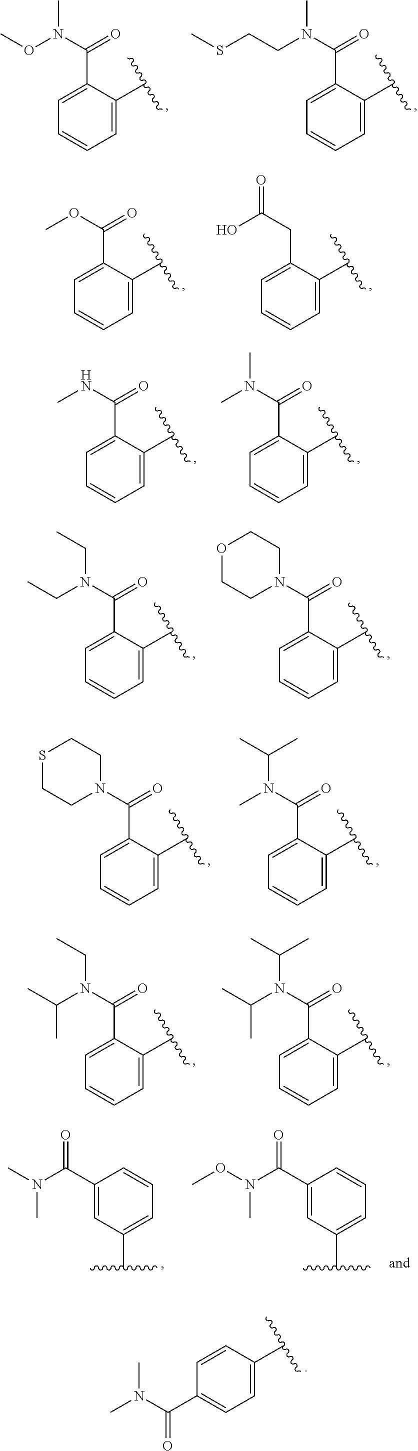

In some embodiments, R^{A} is selected from 6-membered heteroaryl group containing one or two nitrogen atoms, substituted with one or more groups R^{A1}.

In some embodiments, R^{A} is selected from 6-membered heteroaryl group containing one nitrogen atom, substituted with one or more groups R^{A1}.

In some embodiments, R^{A} is selected from 6-membered heteroaryl group containing one or two nitrogen atoms, substituted with one or more groups independently selected from the group consisting of
linear or branchedC₁₋₆alkyl,
—F, —CI, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—NHCOH, —NHCOR^{A2}, —NR^{A2}COH and —NR^{A2}COR^{A2}.

In some embodiments, R^{A} is selected from 6-membered heteroaryl group containing one or two nitrogen atoms, substituted with one or more groups independently selected from the group consisting of
linear or branched C₁₋₆alkyl,
—F, —Cl, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NHR^{A2}, —NR^{A2}₂,
—NHCOH, —NHCOR^{A2}, —NR^{A2}COH and —NR^{A2}COR^{A2}.

In some embodiments, R^{A} is selected from 6-membered heteroaryl group containing one nitrogen atom, substituted with one or more groups independently selected from the group consisting of
linear or branched C₁₋₃alkyl,
—F, —CI, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NHR^{A2}, —NR^{A2}₂,
—NHCOH, —NHCOR^{A2}, —NR^{A2}COH and —NR^{A2}COR^{A2}.

In some embodiments, R^{A} is selected from 6-membered heteroaryl group containing one or two nitrogen atoms, substituted with one or more groups independently selected from the group consisting of
linear or branched C₁₋₃alkyl,
—F, —Cl, —Br, —CN
—OH, —O(C₁₋₃alkyl),
—CF₃, —CF₂H,
—CO(C₁₋₃alkyl),
—CH₂OH, —CH₂O(C₁₋₃alkyl),
—COOH, —COO(C₁₋₃alkyl), —CONH₂, —CONH(C₁₋₃alkyl), —CON(C₁₋₃alkyl)₂,
—OCO(C₁₋₃alkyl), —OCONH₂, —OCONH(C₁₋₃alkyl), —OCON(C₁₋₃alkyl)₂,
—NH₂, —NH(C₁₋₃alkyl), —N(C₁₋₃alkyl)₂,
—NHCOH, —NHCO(C₁₋₃alkyl), —N(C₁₋₃alkyl)COH and —N(C₁₋₃alkyl)CO(C₁₋₃alkyl).

In some embodiments, R^{A} is selected from 6-membered heteroaryl group containing one or two nitrogen atoms, substituted with one or more groups independently selected from the group consisting of
linear or branched C₁₋₃alkyl,
—F, —CI,
—OH, —O(C₁₋₃alkyl),
—CF₃,
—CO(C₁₋₃alkyl),
—CH₂OH, —CH₂O(C₁₋₃alkyl),
—COOH, —COO(C₁₋₃alkyl), —CONH₂, —CONH(C₁₋₃alkyl), —CON(C₁₋₃alkyl)₂,
—NH₂, —NH(C₁₋₃alkyl), —N(C₁₋₃alkyl)₂,
—NHCOH, —NHCO(C₁₋₃alkyl) and —N(C₁₋₃alkyl)COH.

In some embodiments, R^{A} is selected from 6-membered heteroaryl group containing one or two nitrogen atoms, substituted with one or more groups independently selected from the group consisting of
methyl,
—F, —CI,
—OH, —OMe,
—CF₃,
—COMe,
—CH₂OH, —CH₂OMe,
—COOH, —COOMe, —CONH₂, —CONHMe, —CONMe₂,
—NH₂, —NHMe, —NMe₂,
—NHCOH, —NHCOMe and —NMeCOH.

In some embodiments, R^{A} is selected from 6-membered heteroaryl group containing one or two nitrogen atoms, substituted with one or more groups independently selected from the group consisting of
methyl,
—F, —Cl,
—OH, —OMe,
—CF₃,
—COMe,
—CH₂OH, —CH₂OMe,
—COOH and —COOMe,

In some embodiments, R^{A} is selected from 6-membered heteroaryl group containing one or two nitrogen atoms, substituted with one or more groups independently selected from the group consisting of
—COOH, —CON(C₁₋₃alkyl)₂, —COO(C₁₋₃alkyl),
—CONH₂, and
—CF₃.

In some embodiments, R^{A} is selected from 6-membered heteroaryl group containing one or two nitrogen atoms, substituted with one or more groups independently selected from the group consisting of
—COOH, —CON(Me)₂, —CO(Me),
—CONH₂, and
—CF₃.

In some embodiments, R^{A} is selected from 6-membered heteroaryl group containing one or two nitrogen atoms, substituted with one or more groups independently selected from the group consisting of
—CONH₂, and
—CF₃.

In some embodiments, R^{A} is selected from

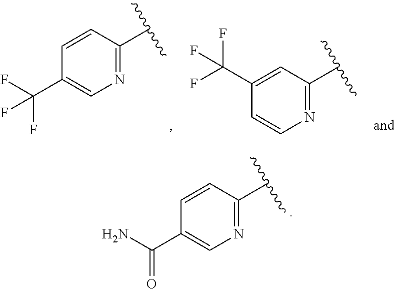 [SMILES: *c1ccc(C(F)(F)F)cn1 ; *c1cc(C(F)(F)F)ccn1 ; *c1ccc(C(N)=O)cn1]

In some embodiments, R^{A} is selected from

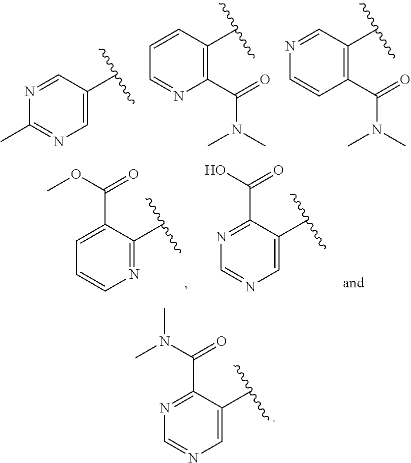

In some embodiments, R^{A} is selected from 8- to 10-membered heterobicyclyl groups containing one or more heteroatoms independently selected from N, O and S.

In some embodiments, R^{A} is selected from 8- to 10-membered heterobicyclyl groups containing one or two heteroatoms independently selected from N, O and S.

In some embodiments, R^{A} is selected from 8- to 10-membered heterobicyclyl groups containing one or two heteroatoms independently selected from N and O.

In some embodiments, R^{A} is selected from 9-membered heterobicyclyl groups containing one or two heteroatoms independently selected from N, O and S.

In some embodiments, R^{A} is selected from 9-membered heterobicyclyl groups containing one or two heteroatoms independently selected from N, O and S, connected to sulfur through a ring carbon atom.

In some embodiments, the heterobicyclyl group is a heteroaromatic group.

In some embodiments, R^{A} is selected from 8- to 10-membered heterobicyclyl groups containing one or more heteroatoms independently selected from N, O and S, wherein the heterobicyclyl group is substituted with one or more groups independently selected from R^{A1}.

In some embodiments, R^{A} is

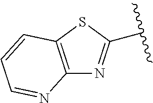 [SMILES: *c1nc2ncccc2s1]

In some embodiments, R^{A} is the group (C1)

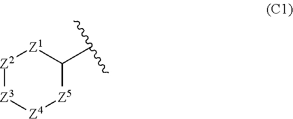

wherein

Z³ is selected from the group consisting of CH₂, CHF and CF₂; one of Z¹, Z², Z⁴ and Z⁵ is selected from the group consisting of
CH₂, CHR^{AL}, CR^{AL}₂,
O,
NH, NR^{A2},
N(CO—R^{A2}), N(CO—NHR^{A2}), N(SO₂—R^{A2}) and N(CO₂—R^{A4}); and
   the remainder of Z¹, Z², Z⁴ and Z⁵ are independently selected from the group consisting of
CH₂, CHR^{AL}, CR^{AL}₂, and
O;
   with the provisos that the ring contains 0 or 1 oxygen atoms, that nitrogen atoms cannot be in a 1,2 or 1,3 relationship to each other, and that when Z¹ or Z⁵ is N, L cannot be a single bond.

In some embodiments, Z³ is selected from the group consisting of CH₂, CHF and CF₂; one of Z¹, Z², Z⁴ and Z⁵ is selected from the group consisting of
CH₂, CHR^{AL} and CR^{AL}₂; and
   the remainder of Z¹, Z², Z⁴ and Z⁵ are CH₂.

In some embodiments, Z³ is selected from the group consisting of CH₂, CHF and CF₂; and the remainder of Z¹, Z², Z⁴ and Z⁵ are CH₂.

In some embodiments, R^{A} is

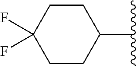 [SMILES: *C1CCC(F)(F)CC1]

In some embodiments, R^{A} is

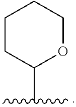 [SMILES: *C1CCCCO1]

In some embodiments, R^{A} is the group (C₂)

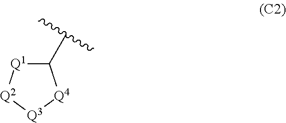

wherein

one of Q¹ to Q⁴ is selected from the group consisting of
O,
NH, NR^{A2},
CH₂, CHR^{AL}, CR^{AL}₂,

N—CO—R^{A2}, N—CO—NHR^{A2}, N—SO₂—R^{A2} and N—CO₂—R^{A4}; and

the remainder of Q¹ to Q⁴ are independently selected from the group consisting of
CH₂, CHR^{AL} and CR^{AL}₂;

with the proviso that the ring contains 0 or 1 oxygen atoms, that the ring contains 0 or 1 nitrogen atoms, and that when Q¹ or Q⁴ is N, L cannot be a single bond.

In some embodiments, one of Q¹ to Q⁴ is selected from the group consisting of
O,
NH, NR^{A2},
CH₂, CHR^{AL} and CR^{AL}₂; and
   the remainder of Q¹ to Q⁴ are independently selected from the group consisting of
CH₂, CHR^{AL} and CR^{AL}₂.

In some embodiments, one of Q¹ to Q⁴ is selected from the group consisting of
CH₂, CHR^{AL} and CR^{AL}₂; and
   the remainder of Q¹ to Q⁴ are CH₂.

In some embodiments, R^{A} is the group (C3)

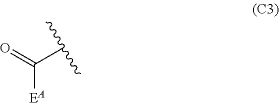

wherein

E^{A} is selected from the group consisting of
—O—R^{A2},
—NH—R^{A2},
—NR^{A2}₂,
—NR^{EA}-E-COR^{EA2} and —NR^{EA1}-E^{A2}-E^{A3}-COR^{EA2};
   wherein E^{A1}, E^{A2} and E^{A3} are D- or L-amino acid residues independently selected from Ala, Asn, Asp, Gln, Glu, Gly, His, Ile, Leu, Met, Phe, Pro, Ser, Thr, Trp, Tyr and Val, wherein the —NR^{EA1}- and —COR^{EA2} groups represent terminals of the alpha or pendent functionality of the amino acids respectively;

the amino acid residues Asp and Glu may form amide bonds from either the alpha or pendent carboxylic acid functionality;

when E^{A1} is Pro, R^{EA1} is absent, otherwise R^{EA1} is R^{E1};

when E^{A2} is Pro, R^{EA1} is absent, otherwise R^{EA1} is R^{E1};

the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{A2} and E^{A3} are present and E^{A3} is not Pro the nitrogen of the amide bond between E^{A2} and E^{A3} may be optionally substituted with R^{E1};

R^{EA2} is selected from —OR^{E7}, —NH₂, —NHR^{A2} and —NR^{A2}R^{E1};

R^{E7} is selected from —H and —R^{A2}; and

R^{E1} is selected from H and linear or branched C₁₋₃alkyl.

In some embodiments, E^{A} is selected from the group consisting of
—O—R^{A2},
—NH—R^{A2},
—NR^{A2}₂, and
—NR^{EA1}-E^{A1}-COR^{EA2}.

In some embodiments, E^{A} is selected from —NR^{EA1}-E^{A1}-COR^{EA2}.

In some embodiments, E^{A} is selected from the group consisting of
—O—R^{A2},
—NH—R^{A2}, and
—NR^{A2}₂.

In some embodiments, R^{EA2} is selected from —OR^{E7}.

In some embodiments, R^{EA2} is selected from —NH₂, —NHR^{A2} and —NR^{A2}R^{E1}.

In some embodiments, R^{EA2} is selected from —NH₂.

In some embodiments, L^{A} is methylene and E^{A} is selected from the group consisting of
—O—R^{A2},
—NH—R^{A2}, and
—N R^{A2}₂.

In some embodiments, L^{A} is methylene and E^{A} is selected from the group consisting of
—NH—R^{A2}, and
—NR^{A2}₂.

In some embodiments, L^{A} is methylene and E^{A} is selected from the group consisting of
—O(C₁₋₃alkyl),
—NH—(C₁₋₃alkyl), and
—N(C₁₋₃alkyl)₂.

In some embodiments, L^{A} is methylene and E^{A} is selected from the group consisting of
—NH—(C₁₋₃alkyl), and
—N(C₁₋₃alkyl)₂.

In some embodiments, L^{A} is methylene and E^{A} is selected from the group consisting of
—NH—CH₃, and
—N(CH₃)₂.

In some embodiments, R^{A} is

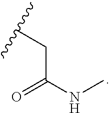 [SMILES: *CC(=O)NC]

In some embodiments, R^{A} is selected from the group (C4)

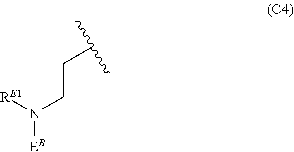

wherein

R^{E1} is selected from H and linear or branched C₁₋₃alkyl;

E^{B} is selected from
E^{BA},
—CO-E^{B1}-NR^{EA}R^{E2}, and
—CO-E^{B2}-E^{B3}-NR^{EB}R^{E2},

wherein E^{B3}, E^{B2} and E^{B3} are D- or L-amino acid residues independently selected from Ala, Asn, Asp, Gln, Glu, Gly, His, Ile, Leu, Met, Phe, Pro, Ser, Thr, Trp, Tyr and Val, wherein the —CO—, —NR^{EA}R^{E2} and —NR^{EB}R^{E2} groups represent terminals of the alpha or pendent functionality of the amino acids;

the amino acid residues Asp and Glu may form amide bonds from either the alpha or pendent carboxylic acid functionality;

when E^{B1} is Pro, R^{EA} is absent, otherwise R^{EA} is —H;

when E^{B3} is Pro, R^{EB} is absent, otherwise R^{EB} is —H;

the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{B2} and E^{B3} are present and E^{B2} is not Pro the nitrogen of the amide bond between E^{B2} and E^{B3} may be optionally substituted with R^{E1};

R^{E2} is selected from —H and —COCH₃; and

when E^{B} is E^{BA}, R^{E1} and E^{BA} together with the nitrogen atom to which they are attached form a group selected from
5- or 6-membered saturated heterocyclyl optionally substituted with one or more groups R^{AL}, and
5- or 6-membered saturated heteroaryl optionally substituted with one or more groups R^{A1}.

In some embodiments, E^{B} is selected from E^{BA}.

In some embodiments, E^{B} is selected from
—CO-E^{B1}-NR^{EA}R^{E2}, and
—CO-E^{B2}-E^{B3}-NR^{EB}R^{E2}.

In some embodiments, E^{B} is —CO-E^{B1}-NR^{EA}R^{E2}.

In some embodiments, R^{E2} is —H.

In some embodiments, R^{E2} is —COCH₃.

In some embodiments of the group (C4), R^{E1} is —H.

In some embodiments of the group (C4), R^{E1} is methyl.

In some embodiments, R^{A} is the group (C₅)

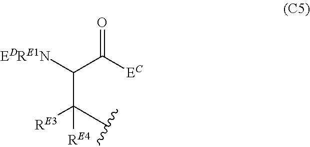

wherein

R^{E1} is selected from H and linear or branched C₁₋₃alkyl;

E^{C} is selected from
—OH,
—OR^{A2}
—NH₂, NHR^{A2}, NR^{A2}₂ and
—NR^{EC1}-E^{C1}-COR^{EC2}
   wherein E^{C1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EC1}— and —COR^{EC2} groups represent terminals of the alpha or pendent functionality of the amino acids;

the amino acid residues Asp and Glu may form amide bonds from either the alpha or pendent carboxylic acid functionality;

when E^{C1} is Pro, R^{EC1} is absent, otherwise R^{EC1} is R^{E1};

the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

R^{EC2} is selected from —OR^{E9}, —NH₂, —NHR^{A2} and —NR^{A2}R^{E1};

R^{E3} and R^{E4} are independently selected from —H and —CH₃;

when R^{E1} is H and E^{C} is —OC₁₋₃alkyl, —NH₂ or —NHC₁₋₃alkyl, E^{D} is selected from
—H, and
—CO-E^{D1}-NR^{ED}R^{E6}
   otherwise, E^{D} is selected from
—R^{E5}, and
—CO-E^{D1}-NR^{ED}R^{E6};
   wherein E^{D1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{ED}R^{E6}- and —CO— groups represent terminals of the alpha or pendent functionality of the amino acids;

wherein the amino acid residues Asp and Glu may form amide bonds from either the alpha or pendent carboxylic acid functionality;

wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;

wherein R^{E5} and R^{E6} are independently selected from —H and —COCH₃;

when E^{D1} is Pro, R^{ED} is absent, otherwise R^{ED} is —H; and

with the proviso that R^{A} is not L-cysteine.

In some embodiments, E^{C} is selected from
—OH,
—OR^{A2}
—NH₂, NHR^{A2}, NR^{A2}₂, and
—NR^{EC1}-E^{C1}-COR^{EC2}; and

E^{D} is selected from
—H, and
—CO-E^{D1}-NR^{ED}R^{E6}.

In some embodiments, E^{C} is selected from
—NH₂, NHR^{A2}, NR^{A2}₂, and
—NR^{EC1}-E^{C1}-COR^{EC2}; and

E^{D} is selected from
—H, and
CO-E^{D1}-NR^{ED}R^{E6}.

In some embodiments, E^{C} is selected from
—NH₂, NHR^{A2}, NR^{A2}₂, and
—NR^{EC1}-E^{C1}-COR_{EC2}; and

E^{D} is —CO-E^{D1}-NR^{ED}R^{E6}.

In some embodiments, E^{C} is —NR^{EC1}-E^{C1}-COR^{EC2}; and

E^{D} is —CO-E^{D1}-NR^{ED}R^{E6}.

In some embodiments, R^{E3} and R^{E4} are the same.

In some embodiments, R^{E3} and R^{E4} are both —H.

In some embodiments, R^{E3} and R^{E4} are both methyl.

In some embodiments of the group (C₅), R^{E1} is —H.

In some embodiments, R^{A} is

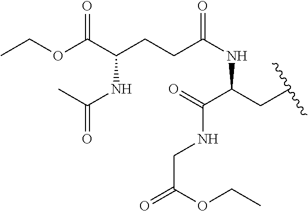 [SMILES: *C[C@H](NC(=O)CC[C@H](NC(C)=O)C(=O)OCC)C(=O)NCC(=O)OCC]

In some embodiments, R^{A} is the group (C6)

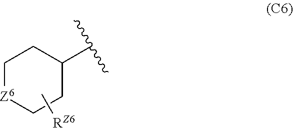

wherein

Z⁶ is selected from N—CO—R^{A2} and N—CO—NHR^{A2}; and

R^{Z6} is one or two optional methyl substituents.

According to another aspect of the invention, the following compounds are provided:

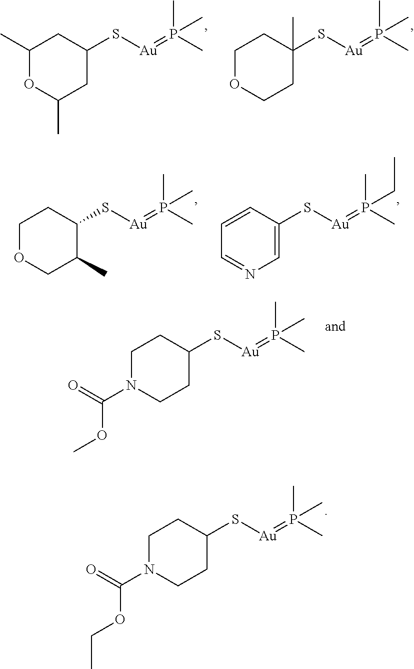

According to another aspect of the invention, the following compounds are provided:

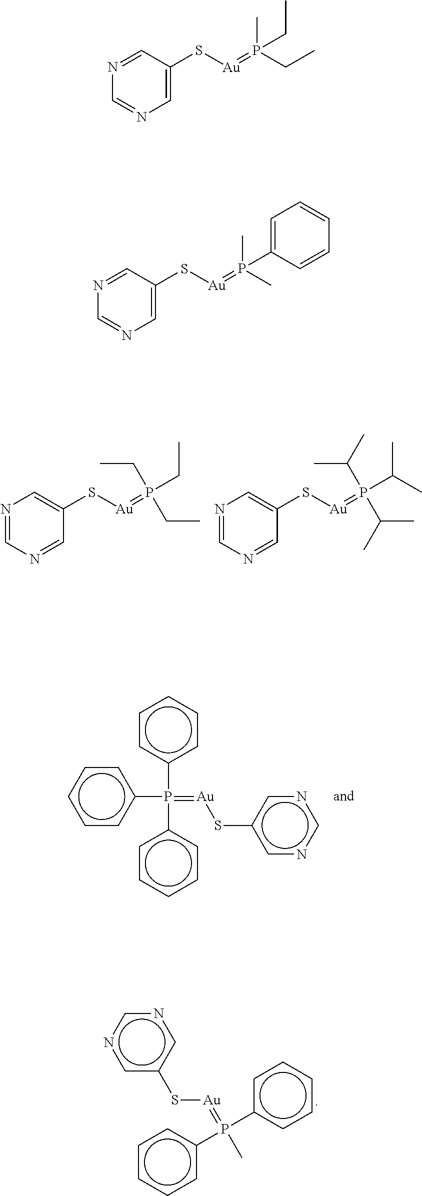

In a further aspect, the invention provides the following compounds for use in the prevention or treatment of a bacterial infection. Another aspect is the use of the following compounds in the manufacture of a medicament for the prevention or treatment of a bacterial infection. Another aspect is a method of preventing or treating a bacterial infection in a human or animal, comprising administering to said patient an effective amount of a pharmaceutical composition containing one of the following compounds. Another aspect may relate to the treatment of fungal infection, e.g. by providing one of the following compounds for use in the prevention or treatment of a fungal infection.

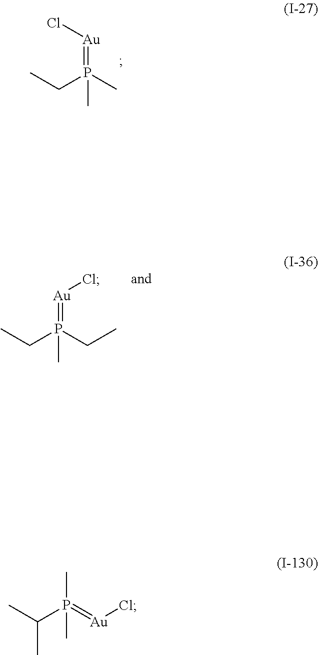

Particular embodiments of the invention are shown in the examples.

Bacterial Infections

Bacteria that cause infection of humans include, but are not limited to, those set out below in Table 1.

TABLE 1

| | | Gram |
|---|---|---|
| Genus | Important species | negative/positive |
| Bordetella | Bordetella pertussis | Gram-negative |
| Borrelia | Borrelia burgdorferi | Gram-negative |
| Brucella | Brucella abortus | Gram-negative |
| | Brucella canis | |
| | Brucella melitensis | |
| | Brucella suis | |
| Burkholderia | Burkholderia cepacia | Gram-negative |
| Campylobacter | Campylobacter jejuni | Gram-negative |
| Chlamydia and | Chlamydia pneumoniae | (not |
| Chlamydophila | Chlamydia trachomatis | Gram-stained) |
| | Chlamydophila psittaci | |
| Clostridium | Clostridium botulinum | Gram-positive |
| | Clostridium difficile | |
| | Clostridium perfringens | |
| | Clostridium tetani | |
| Corynebacterium | Corynebacterium diphtheriae | Gram-positive |
| Enterobacter | Enterobacter cloacae | Gram-negative |
| Enterococcus | Enterococcus faecalis | Gram-positive |
| | Enterococcus faecium | |
| Escherichia | Escherichia coli | Gram-negative |
| Francisella | Francisella tularensis | Gram-negative |
| Haemophilus | Haemophilus influenzae | Gram-negative |
| Helicobacter | Helicobacter pylori | Gram-negative |
| Klebsiella | Klebsiella oxytoca | Gram-negative |
| | Klebsiella pneumoniae | |
| Legionella | Legionella pneumophila | Gram-negative |
| Leptospira | Leptospira interrogans | Gram-negative |
| Listeria | Listeria monocytogenes | Gram-positive |
| Moraxella | Moraxella catarrhalis | Gram-negative |
| Mycobacteriae | Mycobacterium tuberculosis | Gram-indeterminate |
| Neisseria | Neisseria gonorrhoeae | Gram-negative |
| | Neisseria meningitidis | |
| Proteus | Proteus vulgaris | Gram-negative |
| Pseudomonas | Pseudomonas aeruginosa | Gram-negative |
| Rickettsia | Rickettsia rickettsii | Gram-negative |
| Salmonella | Salmonella typhi | Gram-negative |
| | Salmonella typhimurium | |
| Shigella | Shigella sonnei | Gram-negative |
| Staphylococcus | Staphylococcus aureus | Gram-positive |
| | Staphylococcus epidermidis | |
| | Staphylococcus saprophyticus | |
| Streptococcus | Streptococcus agalactiae | Gram-positive |
| | Streptococcus pneumoniae | |
| | Streptococcus pyogenes | |
| Treponema | Treponema pallidum | Gram-negative |
| Vibrio | Vibrio cholerae | Gram-negative |
| Yersinia | Yersinia pestis | Gram-negative |
| | Yersinia enterocolitica | |
| | Yersinia pseudotuberculosis | |

The bacterial infection prevented and/or treated by compounds of the present invention may be infection by one or more Gram-positive bacteria. Furthermore, the compounds of the present invention may be selective for one or more Gram-positive bacteria over Gram-negative bacteria. Thus, compounds of the present invention may show no significant inhibition of growth of Gram-negative bacteria.

The bacterial infection prevented and/or treated by compounds of the present invention may be infection by one or more Gram-negative bacteria. Furthermore, the compounds of the present invention may be selective for one or more Gram-negative bacteria over Gram-positive bacteria. Thus, compounds of the present invention may show no significant inhibition of growth of Gram-positive bacteria.

Furthermore, the compounds of the present invention may inhibit the growth of both Gram-positive bacteria and Gram-negative bacteria.

Therapeutic index is the ratio of the dose that produces growth inhibition in 50% of CHO or HepG22 cells divided by the dose where 50% of S.aureus growth is inhibited. In some embodiments, compounds have a therapeutic index of greater than 1. In other embodiments, compounds have a therapeutic index of greater than 4. In other embodiments, compounds have a therapeutic index of greater than 8.

Representative examples of Gram-positive bacteria include Staphylococcus (e.g. S. aureus, S. epidermis), Enterococci (e.g. E. faecium, E. faecalis), Clostridia (e.g. C. difficile), Propionibacteria (e.g. P. acnes) and Streptococcus.

Bacterial infections in animals are, for example, described in “Pathogenesis of Bacterial Infections in Animals”, edited by Carlton L. Gyles, John F. Prescott, J. Glenn Songer, and Charles O. Thoen, published by Wiley-Blackwell (Fourth edition, 2010—ISBN 978-0-8138-1237-3), which is hereby incorporated by reference. Many are the same as listed above for humans.

Combinations

Treatments as described herein may be in combination with one or more known antibiotics, examples of which are described below:

(a) Aminoglyosides: Amikacin, Gentamicin, Kanamycin, Neomycin, Netilmicin, Tobramycin, Paromomycin, Streptomycin; Spectinomycin;

(b) Ansamycins: Geldanamycin, Herbimycin, Rifaximin;

(c) Carbacephem:Loracarbef;

(d) Cabapenems: Ertapenem, Doripenem, Imipenem/Cilastatin, Meropenem;

(e)1^{st} generation Cephlasporins: Cefadroxil, Cefazolin, Cefalotin or Cefalothin, Cefalexin;

(f) 2^{nd} generation Cephlasporins: Cefaclor, Cefamandole, Cefoxitin, Cefprozil, Cefuroxime;

(g) 3^{rd} generation Cephlasporins: Cefixime, Cefdinir, Cefditoren, Cefoperazone, Cefotaxime, Cefpodoxime, Ceftazidime, Ceftibuten, Ceftizoxime, Ceftriaxone;

(h) 4^{th} generation Cephlasporins: Cefepime;

(i) 5^{th} generation Cephlasporins: Ceftaroline fosamil, Ceftobiprole, Ceftolozane-tazobactam, Ceftaroline;

(j) Glycopeptides: Teicoplanin, Vancomycin, Telavancin, Dalbavancin, Oritavancin;

(k) Lincosamides: Clindamycin, Lincomycin

(I) Lipopeptide: Daptomycin

(m) Macrolides: Azithromycin, Clarithromycin, Dirithromycin, Erythromycin, Roxithromycin, Troleandomycin, Telithromycin, Spiramycin, Rifabutin; Fidaxomicin;

(n) Monobactams: Aztreonam;

(o) Nitrofurans: Furazolidone, Nitrofurantoin;

(p) Oxazolidonones: Linezolid, Posizolid, Radezolid, Torezolid, Tedizolid, Tedizolid phosphate;

(q) Penicillins: Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin;

(r) Polypeptides: Bacitracin, Colistin, Polymyxin B, Polymyxin E (colistin);

(s) Quinolones: Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin;

(t) Sulfonamides: Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine;

(u) Tetracylines: Demeclocycline, Doxycycline, Minocycline, Oxytetracycline, Tetracycline;

(v) Antibodies: bezlotoxumab;

(w) Non-β-lactam β-lactamase inhibitors: avibactam;

(x) Quinolines: Bedaquiline; and

(y) Combinations: ceftazidime-avibactam, colistin-ceftazidime, colistin-rifabutin.

General Experimental

The invention also provides a process for the preparation of a compound of formula II:

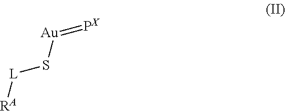

which comprises reacting a compound of general formula III, IV, V, VI or IX:

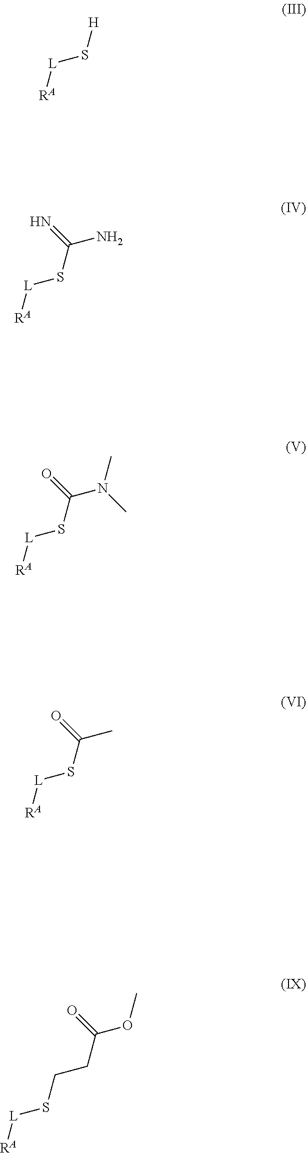

with chloro(trialkyl phosphine) gold(I) complexes of general formula VII:

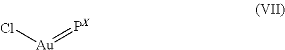

Compounds of Formula (I) can be synthesised in an analogous manner.

Isomers, Salts and Solvates

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

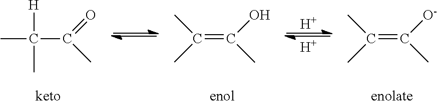

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; Au may be in any isotopic forms, including ¹⁹⁷Au and ¹⁹⁵Au; S may be in any isotopic forms, including ³²S, ³³S, ³⁴S and ³⁶S; P may be in any isotopic forms, including ³¹P, ³³P and ³²P; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., −NH₂ may be −NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also include salt forms thereof.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

Dosage and Formulation

The dosage administered to a patient will normally be determined by the prescribing physician and will generally vary according to the age, weight and response of the individual patient, as well as the severity of the patient's symptoms and the proposed route of administration. However, in most instances, an effective therapeutic daily dosage will be in the range of from about 0.05 mg/kg to about 100 mg/kg of body weight and, preferably, of from 0.05 mg/kg to about 5 mg/kg of body weight administered in single or divided doses. In some cases, however, it may be necessary to use dosages outside these limits.

While it is possible for an active ingredient to be administered alone as the raw chemical, it is preferable to present it as a pharmaceutical formulation. The formulations, both for veterinary and for human medical use, of the present invention comprise a compound of formula (I) in association with a pharmaceutically acceptable carrier therefore and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration. The toxicity of certain of the compounds in accordance with the present invention will preclude their administration by systemic routes, and in those, and other, cases opthalmic, topical or buccal administration, and in particular topical administration, is preferred for the treatment of local infection.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomel formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent to hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200 μm.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Especially valuable are liquid non-ionic surfactants and solid anionic surfactants or mixtures thereof. The liquid non-ionic surfactant may constitute from 0.01 up to 20% w/w of the formulation, though preferably it constitutes below 1% w/w of the formulation. The solid anionic surfactants may constitute from 0.01 up to 20% w/w of the formulation, though preferably below 1% w/w of the composition.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Embodiments of the invention will now be described by way of example only.

### EXAMPLES

Analytical Methods

Analysis of products and intermediates has been carried out using reverse phase analytical HPLC-MS using the parameters set out below.

HPLC Analytical Methods:

AnalpH2_MeOH_4min: Phenomenex Luna C18 (2) 3 μm, 50×4.6 mm; A=water+0.1% formic acid; B=MeOH+0.1% formic acid; 45° C.; % B: 0.0 min 5%, 1.0 min 37.5%, 3.0 min 95%, 3.5 min 95%, 3.51 min 5%, 4.0 min 5%; 2.25 mL/min.

Preparative HPLC Methods

Reverse Phase Preparative HPLC-MS: Mass-directed purification by preparative LC-MS using a preparative C-18 column (Phenomenex Luna C18 (2), 100×21.2 mm, 5 μm).

Generic Acidic Conditions:

A=water +0.1% formic acid; B=MeOH+0.1% formic acid; 20° C.; % B: 0.0 min Initial between 2% and 50%, 0.1 min % as per Initial, 7.0 min between 40% and 95%, 9.0 min 95%, 10.0 min 95%, 10.1 min back to Initial %; 12.0 min Initial %; 20.0 mL/min.

Generic Basic Conditions:

A=water pH 9 (Ammonium Bicarbonate 10 mM); B=MeOH; 20° C.; % B: 0.0 min Initial between 2% and 50%, 0.1 min % as per Initial, 7.0 min between 40% and 95%, 9.0 min 95%, 10.0 min 95%, 10.1 min back to Initial %; 12.0 min Initial %; 20.0 mL/min.

NMR was also used to characterise final compounds. NMR spectra were obtained Bruker Advance 400 or Bruker DRX 400 at room temperature unless otherwise stated. 1H NMR spectra are reported in ppm and referenced to either tetramethylsilane (0.00 ppm), DMSO-d6 (2.50 ppm), CDCl₃ (7.26 ppm) or CD₃OD (3.31 ppm).

Abbreviations Used

For the examples below as well as throughout the application, the following abbreviations have the following meanings. If not defined, the terms have their generally accepted meanings.

° C. Degrees Centigrade

Ac Acetyl

app Apparent

aq. Aqueous

br Broad

d Doublet

DABCO 1,4-Diazabicyclo[2,2,2]octane

DCM Dichloromethane

DIPEA N,N-Diisopropylethylamine

DMA Dimethylacetamide

DMF Dimethylformamide

DMSO Dimethyl sulfoxide

Et Ethyl

EtOAc Ethyl acetate

EtOH Ethanol

Et₂O Diethyl ether

FA Formic acid

g Gram

h Hour(s)

HATU 1-[Bis(dimethylamino)methylene]-1H-1,2,3-triazolo[4,5-b]pyridinium 3-oxid hexafluorophosphate

HMPA Hexamethylphosphoramide

HPLC High-performance liquid chromatography

ⁱPr Isopropyl

J Coupling constant

LC-MS Liquid chromatography-mass spectrometry

Me Methyl

MeCN Acetonitrile

MeOH Methanol

mg Milligram

min Minute(s)

mL Millilitre

mmol Millimole

Ms Mesyl

O/N Overnight

ppm Parts per million

ppt Precipitate

q Quartet

quint Quintet

rt Room temperature

Rochelle Salt Potassium sodium tartrate tetrahydrate

s Singlet

TCEP.HCl Tris(2-carboxyethyl)phosphine hydrochloride

TEA Triethylamine

TFA Trifluoroacetic acid

THF Tetrahydrofuran

TLC Thin layer chromatography

TMS Trimethylsilyl

t Triplet

WIPE Water/isopropanol/Ethyl acetate (1:2:9)

XantPhos 4,5-Bis(diphenylphosphino)-9,9-dimethylxanthene

Synthesis of Key Intermediates

A number of the requisite precursors III, IV, V, VI, and IX, necessary for coupling with gold(I) phosphine chloride complexes VII, required synthesis from commercial starting materials. Other precursors used were commercially available.

(S)-2-Acetylamino-4-[(R)-1-(ethoxycarbonylmethyl-carbamoyl)-2-mercapto-ethylcarbamoyl]-butyric acid ethyl ester I-3

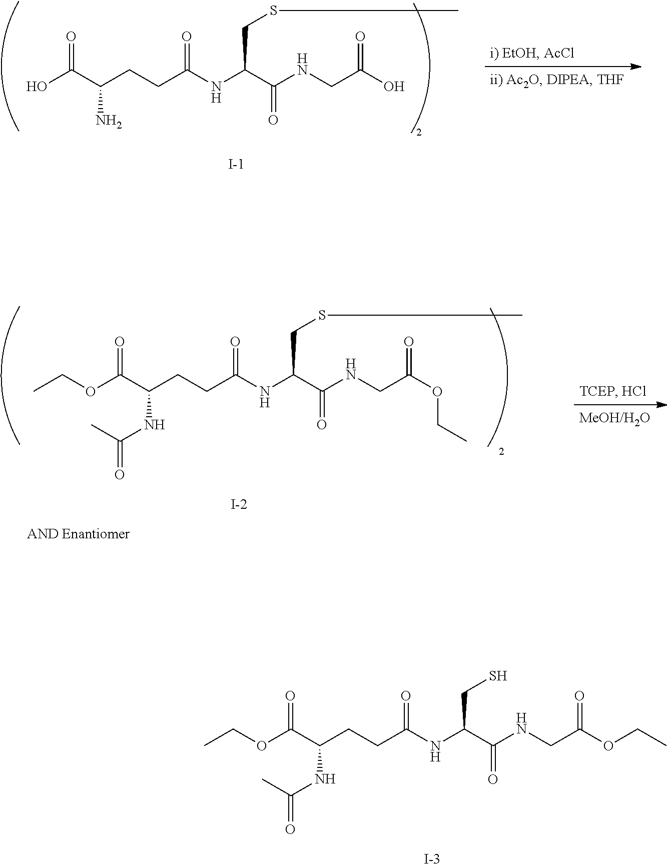

(a) (4S,9R,14R,19S)-ethyl19-acetamido-9,14-bis((2-ethoxy-2-oxoethyl)carbamoyl)-4-(ethoxycarbonyl)-2,7,16-trioxo-11,12-dithia-3,8,15-triazaicosan-20-oate I-2

Oxidised L-glutathione I-1 (980 mg, 1.6 mmol) was suspended in dry EtOH (40 mL). The reaction mixture was cooled to 0° C. and AcCl (6.82 mL, 96 mmol) was added dropwise over 5 min. The reaction mixture was heated to 50° C. O/N, followed by evaporation to dryness to afford a pink crude solid. The crude material was resuspended in dry THF (50 mL), DIPEA (600 μL, 3.44 mmol) was added, followed by dropwise addition of acetic anhydride (4.9 mL, 51.84 mmol). The reaction mixture was stirred at rt O/N. The solvent was evaporated and the crude material was purified by preparative HPLC (acidic conditions) to afford the title compound (90 mg, 0.11 mmol, 7%).

(b) (4S,9R,14R,19S)-ethyl19-acetamido-9,14-bis((2-ethoxy-2-oxoethyl)carbamoyl)-4-(ethoxycarbonyl)-2,7,16-trioxo-11,12-dithia-3,8,15-triazaicosan-20-oate I-3 I-2 (90 mg, 0.11 mmol) was dissolved in a mixture of MeOH (0.5 mL) and water (0.5 mL). TCEP (165 mg, 0.58 mmol) was added and the reaction mixture was stirred at rt O/N. The solvent was evaporated, the residual solid was dissolved in water and extracted with DCM (3×). The combined organic fractions were dried and evaporated to yield the title compound as a white solid (90 mg, 0.11 mmol, quantitative).

2-(1-Methyl-1H-tetrazol-5-ylmethyl)-isothiourea I-6

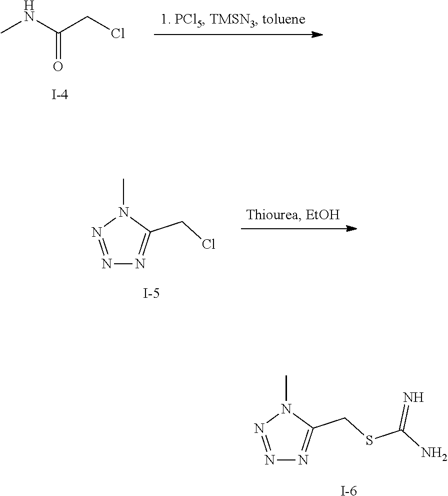

(a) 5-Chloromethyl-1-methyl-1H-tetrazole I-5

N-Methylchloroacetamide I-4 (1.0 g, 9.3 mmol) was dissolved in dry toluene (30 mL). PCl₅ (2.13 g, 10.23 mmol) was added in one portion and the reaction mixture was stirred at rt for 1 h under an atmosphere of N₂. Trimethylsilyl azide (1.85 mL, 13.95 mmol) was added dropwise over 15 min and the resulting reaction mixture was stirred at rt O/N. The reaction mixture was diluted with EtOAc and washed with water, 2M NaOH (aq.) and brine. The organic fraction was concentrated to dryness and purified by flash column chromatography (Biotage Isolera Four, 25 g KP-Sil column eluting with a gradient from isohexane to EtOAc) to yield the desired product (283 mg, 2.14 mmol, 23%).

(b) 2-(1-Methyl-1H-tetrazol-5-ylmethyl)-isothiourea I-6

A mixture of 5-chloromethyl-1-methyl-1H-tetrazole I-5 (240 mg, 1.81 mmol) and thiourea (138 mg, 1.81 mmol) in EtOH (10 mL) was heated to reflux O/N. The formation of a white ppt was observed. The reaction mixture was cooled to rt, the ppt filtered and dried under high vacuum O/N to afford the title compound (311 mg, 1.81 mmol, quantitative).

1 H-Tetrazole-5-thiol I-8

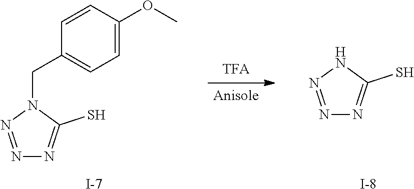

1-[(4-Methoxyphenyl)methyl]-1H-1,2,3,4-tetrazole-5-thiol I-7 (200 mg, 0.90 mmol) was dissolved in a mixture of TFA (1.7 mL) and anisole (0.3 mL). The reaction mixture was heated to 100° C. for 2 h in a microwave reactor. A white ppt had formed, which was filtered, triturated with TFA (2×1 mL) and dried under high vacuum O/N to afford the title compound as a white solid (60 mg, 0.59 mmol, 65%).

6-Mercapto-nicotinamide 1-10

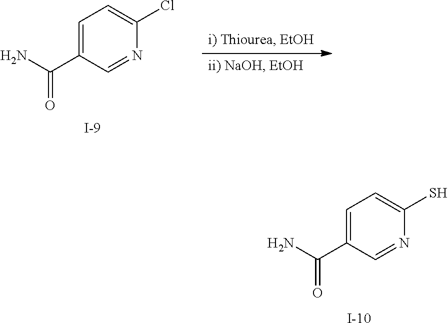

6-Chloronicotinamide 1-9 (400 mg, 2.55 mmol) and thiourea (214 mg, 2.81 mmol) were suspended in EtOH (30 mL) and the reaction mixture heated at reflux for 2 days. A yellow ppt had formed, which was filtered and dried. LC-MS analysis (AnalpH2_MeOH_4min) of the ppt indicated partial hydrolysis to the thiol. As a consequence, the ppt was suspended in a mixture of EtOH (30 mL) and aq. NaOH and the reaction mixture heated to reflux O/N. The reaction mixture was evaporated to dryness, redissolved in water, acidified to pH1 with conc. aq. HCl and extracted with DCM (3×). A yellow solid precipitated in the aqueous fraction which was filtered and triturated with MeOH (3 ×) to yield the desired product as a yellow solid (10 mg, 0.06 mmol, 3%).

S-[3-(Methylsulfonyl)phenyl]carbamothioic acid dimethyl ester I-13

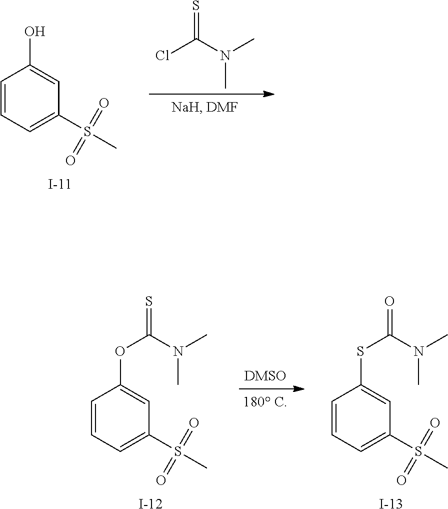

(a) O-[3-(Methylsulfonyl)phenyl]carbamothioic acid dimethyl ester I-12

3-Methanesulfonylphenol I-11 (1.0 g, 5.81 mmol) was dissolved in dry DMF (10 mL). NaH (60% dispersion in mineral oil, 255 mg, 6.39 mmol) was added at which point effervescence was observed and the reaction mixture was stirred at rt for 10 min. N,N-Dimethylthiocarbamoyl chloride (790 mg, 6.39 mmol) was added and the reaction mixture was heated to 80° C. for 1 h, cooled to rt and stirred O/N. The reaction mixture was poured into brine and was extracted with DCM several times. The combined organic fractions were dried, evaporated and purified by flash column chromatography (Biotage Isolera Four, 25 g KP-Sil column eluting with a gradient from isohexane to 40% EtOAc/isohexane) to afford the title compound (1.42 g). This was used crude [80% by LC-MS (AnalpH2_MeOH_4min)] in the successive step.

(b) S-[3-(Methylsulfonyl)phenyl]carbamothioic acid dimethyl ester I-13

O-[3-(Methylsulfonyl)phenyl]carbamothioic acid dimethyl ester I-12 (crude 490 mg, 1.51 mmol based on 80% purity) was dissolved in DMSO (11 mL). The reaction was heated to 180° C. for 4 h in a microwave reactor. Purification was carried out by preparative HPLC (acidic conditions) to afford the title compound (20 mg, 0.08 mmol, 5%).

3, 4-Diacetoxybenzenethiol I-17

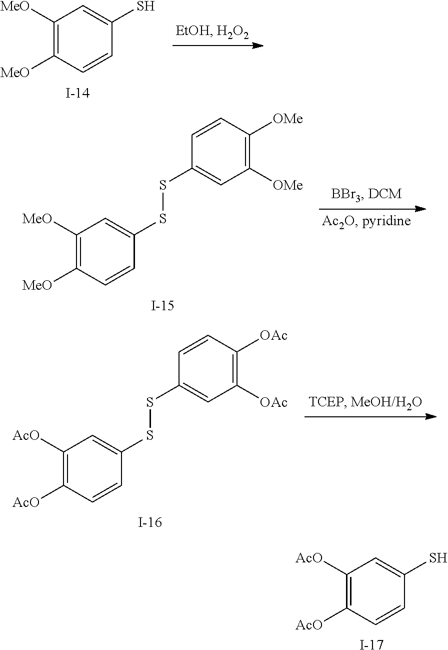

(a) 4-[(3,4-Dimethoxyphenyl)disulfanyl]-1,2-dimethoxy-benzene I-15

To a solution of 3,4-dimethoxybenzenethiol I-14 (1.0 g, 6.3 mmol) in EtOH (10 mL) was added 5 drops of 35% hydrogen peroxide solution under vigorous stirring. After stirring at rt for 18 h, the resulting precipitate was collected and washed with cold EtOH to afford the title compound as an off white solid (600 mg, 1.77 mmol, 56%).

(b) 4-[(3,4-Acetoxyphenyl)disulfanyl]-1,2-dimethoxy-benzene I-16

To a solution of 4-[(3,4-dimethoxyphenyl)disulfanyl]-1,2-dimethoxy-benzene I-15 (356 mg, 1.05 mmol) in dry DCM (20 mL) at 0° C. was added dropwise a 1M solution of boron tribromide in DCM (6.3 mL, 6.3 mmol). The reaction mixture was stirred for 1 h at 0° C., followed by 1 h at rt, and was then adsorbed onto silica and purified by column chromatography (Biotage SP1, 10 g KP-Sil column, 30% EtOAc/isohexane to 80% EtOAc/isohexane) to afford a brown oil (83 mg, 28%) which was used directly. The brown oil was solubilised in pyridine (30 4). Acetic anhydride (65 μL, 0.66 mmol) was added and the resulting mixture was heated for 3 h at 60° C. Upon cooling to rt, the reaction mixture was diluted with DCM (15 mL), washed with water (2×15 mL) and brine, before passing through a phase separator cartridge (Biotage) and concentrated in vacuo. The residue was purified by column chromatography (Biotage SP1, 10 g KP-Sil column, 25% EtOAc/isohexane to 50% EtOAc/isohexane) to afford the title compound as a brown oil (115 mg, 0.26 mmol, 24% over two steps).

(c) 3,4-Diacetoxybenzenethiol I-17

To a solution of 4-[(3,4-acetoxyphenyl)disulfanyl]-1,2-dimethoxy-benzene I-16 (37 mg, 0.081 mmol) in MeOH/water 1:1 (1 mL) was added TCEP.HCl (116 mg, 0.41 mmol). The resulting reaction mixture was stirred for 18 h at rt and concentrated under reduced pressure. The residue was dissolved in water (10 mL) and extracted with DCM (3×10 mL). The organics extracts were combined, washed with brine and passed through a phase separator cartridge (Biotage) and concentrated in vacuo to afford the title compound as a pink oil (11 mg, 0.05 mmol, 60%).

4-Sulfanylbenzene-1, 2-diol I-19

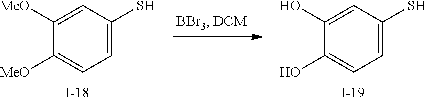

To a solution of 3,4-dimethoxybenzenethiol I-18 (150 mg, 0.66 mmol) in dry DCM (10 mL) at 0° C. was added dropwise a 1M solution of boron tribromide in DCM (2 mL, 2 mmol). The reaction mixture was stirred for 1 h at 0° C., followed by 1 h at rt. The reaction mixture was diluted with DCM (10 mL), washed with water (2×10 mL), brine (10 mL), passed through a phase separator cartridge (Biotage) and concentrated in vacuo to afford a ca. 2:1 (by NMR) mixture of the title compound and the corresponding disulfide as a pink oil (48 mg).

Thioacetic acid 4,4-difluoro-cyclohexylester I-23

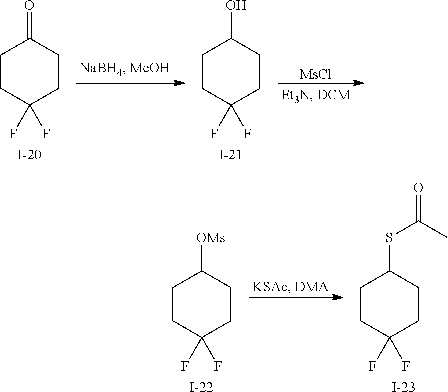

(a) 4,4-Difluoro-cyclohexanol I-21

To a solution of 4,4-difluoro-cyclohexanone I-20 (205 mg, 1.53 mmol) in MeOH (4 mL) at 0° C. was added sodium borohydride (116 mg, 3.0 mmol). The reaction mixture was stirred at 0° C. for 3 h. The reaction was quenched with saturated aq. ammonium chloride (5 mL), MeOH was removed in vacuo and the aqueous layer was extracted with DCM (3×10 mL). The combined organic fractions were passed through a phase separator cartridge (Biotage) and concentrated in vacuo to afford the crude title compound as a colourless oil (216 mg)).

(b) Methanesulfonic acid 4,4-difluoro-cyclohexyl esterI-22

To a solution of 4,4-difluoro-cyclohexanol I-21 (216 mg) in DCM (5 mL) was added mesyl chloride (148 μL, 1.9 mmol) and triethylamine (442 μL, 3.1 mmol). The reaction mixture was stirred at 0° C. for 2 h. The reaction was quenched with water (5 mL), the layers separated and the aqueous layer extracted with DCM (3×5 mL). The combined organic extracts were washed with saturated aq. sodium bicarbonate (10 mL) and brine (10 mL), passed through a phase separator cartridge (Biotage) and concentrated in vacuo to afford the title compound as a yellow oil (308 mg, 1.44 mmol, 94% over 2 steps).

(c) Thioacetic acid 4,4-difluoro-cyclohexylester I-23

To a solution of methanesulfonic acid 4,4-difluoro-cyclohexyl ester I-22 (82 mg, 0.38 mmol) in DMA (2 mL) was added potassium thioacetate (131 mg, 1.1 mmol). The reaction was heated at 80° C. for 18 h. The reaction was cooled to rt, Et₂O (10 mL) and water (10 mL) were added. The layers were separated and the aqueous layer extracted with Et₂O (3×10 mL). The combined organic extracts were washed with water (10 mL) and brine (10 mL) before passing through a phase separator cartridge (Biotage). The crude residue was purified by column chromatography (Biotage SP1, 25 g KP-Sil, eluting with a gradient of isohexane to EtOAc) to afford the title compound as a pale yellow oil (42 mg, 0.21 mmol, 57%).

Several of the requisite chloro(trialkyl phosphine) gold(I) complexes VII, necessary for coupling with precursors III, IV, V, VI and IX required synthesis from commercial starting materials:

Dimethylethylphosphine gold(I) chloride I-27

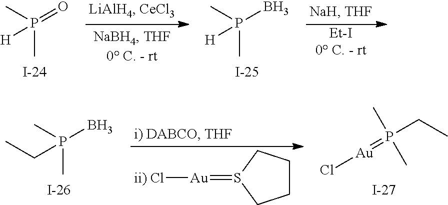

(a) Dimethylphosphine borane I-25

Cerium(III) chloride (25 g, 101.4 mmol) was suspended in THF (100 mL) and stirred at rt for 1 h. Sodium borohydride (3.8 g, 101.4 mmol) was then added and the suspension stirred at rt for a further 1 h. The reaction was cooled to 0° C. at which point dimethylphosphine oxide I-24 (2.6 g, 33.8 mmol) was added dropwise followed by lithium aluminium hydride (1M in THF, 40.7 mL, 40.7 mmol) also dropwise. The reaction was stirred at rt O/N before diluting with toluene (50 mL) then quenching with water (25 mL) and aqueous HCl (6N, 25 mL). The suspension was filtered through celite and the layers separated. The aqueous phase was extracted with DCM (3×40 mL) and the combined organic extracts washed with brine (1×40 mL) and passed through a phase separator cartridge (Biotage). Concentration in vacuo gave the crude product as a yellow oil which was purified by column chromatography (Biotage Isolera Four, 25 g KP-Sil column eluting with a gradient of isohexane to 20% EtOAc/isohexane) to provide the title compound as a colourless oil (1.49 g, 19.6 mmol, 58%).

(b) Dimethylethylphosphine borane I-26

Dimethylphosphine borane I-25 (100 mg, 1.3 mmol) was dissolved in THF (3 mL) and the colourless solution cooled to 0° C. NaH (60% dispersion in mineral oil, 53 mg, 1.3 mmol) was added in one portion, whereupon effervescence was observed. The opaque reaction was stirred at rt for 10 min then cooled to 0° C. whereupon iodoethane (0.12 mL, 1.4 mmol) was added in one portion. When TLC had indicated completion of the reaction, water (10 mL) and Et₂O (10 mL) were added and the phases separated. The aqueous phase was extracted with Et₂O (2×15 mL) and the combined organic extracts washed with brine (1×20 mL) before passing through a phase separator cartridge (Biotage). Concentration in vacuo gave the crude product as a colourless gum. Purification by column chromatography (Biotage Isolera Four, 10 g KP-Sil column, eluting with a gradient of isohexane to 20% EtOAc/isohexane) provided the title compound as a white solid (122 mg, 1.1 mmol, 90%).

(c) Dimethylethylphosphine gold(I) chloride I-27

Dimethylethylphosphine borane I-26 (225 mg, 2.0 mmol) was dissolved in THF (5 mL) and the colourless solution degassed with nitrogen for 5 min. DABCO (640 mg, 6.0 mmol) was added and the reaction sealed with a Teflon screw cap. The reaction was heated to 100° C. and stirred at this temperature for 4 h before cooling in an ice bath and adding a solution of chloro(tetrahydrothiophene)gold(I) (640 mg, 2.0 mmol) in 5 mL dry DCM. After stirring at rt O/N the reaction was diluted with DCM (10 mL) and water (10 mL) and the phases separated. The aqueous phase was extracted with DCM (2×20 mL) and the combined organic extracts washed with brine (20 mL) before passing through a phase separator cartridge (Biotage). Concentration in vacuo gave the crude product as a brown oil which was purified by column chromatography (Biotage SP1, 25 g KP-Sil eluting with 25% EtOAc/isohexane to 60% EtOAc/isohexane) to provide the title compound as a white solid (265 mg, 0.82 mmol, 41%). ¹H-NMR (400 MHz, CDCl₁₃): δ ppm 1.85 (2H, dq, J=10.9, 7.6 Hz), 1.57 (6H, d, J=11.1 Hz), 1.26 (3H, dt, J=20.5, 7.6 Hz). ³¹P-NMR (162 MHz, CDCl₃): δppm 4.07 (s).

1-Methylphospholane gold(I) chloride I-30 and 1-methylphosphinane gold(I) chloride I-31

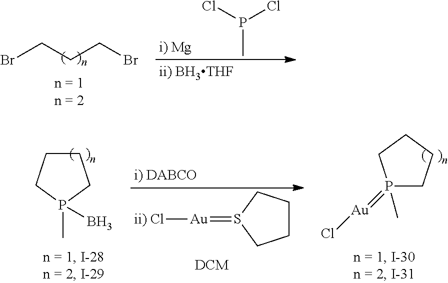

(a) 1-Methylphospholaneborane I-28

The bis-Grignard reagent was prepared by treating magnesium (1.0 g, 0.04 mol) with 1,4-dibromobutane (4.3 g, 20 mmol) in dry THF (50 mL) at 65° C. for 3 h. The reaction mixture was cooled to 0° C. before adding a cooled (10° C.) solution of dichloromethyl phosphine (2.3 g, 20 mmol) in dry THF (25 mL) dropwise maintaining a temperature of 10° C. The mixture was stirred O/N at rt. Borane-THF complex (1.0 M, 20 mL, 20 mmol) was added dropwise and the reaction mixture stirred for additional 4 h. The reaction mixture was poured onto a mixture of ice (200 g) and aqueous HCl (2M, 100 mL) with vigorous stirring. The aqueous phase was extracted with DCM (3×100 mL) and the combined organic extracts dried over MgSO₄. Concentration in vacuo gave the crude product as a yellow oil which was purified by column chromatography (Biotage SP1, 50 g KP-Sil column, eluting with isohexane to DCM) to provide the title compound as a colourless oil (700 mg, 6.0 mmol, 30%).

(b) 1-Methylphosphinaneborane I-29

Procedure similar to that described for 1-methylphospholaneborane I-28 starting from 1,5-dibromopentane (4.6 g, 20 mmol) to provide the title compound as a colourless oil (546 mg, 4.2 mmol, 21%).

(c) 1-Methylphospholane gold(I) chloride I-30

Procedure similar to that described for dimethylethylphosphine gold(I) chloride I-27 starting from 1-methylphospholaneborane 1-28 (116 mg, 1.0 mmol) to provide the title compound as an off-white solid (200 mg, 0.6 mmol, 60%). ¹H-NMR (400MHz, CDCl₃): δ ppm 2.35-2.19 (2H, m), 2.03-1.85 (6H, m), 1.55 (3H, d, J=10.6 Hz). ³¹P-NMR (162 MHz, CDCl₃): δ ppm 11.82 (s).

(d) 1-Methylphosphinane gold(I) chloride I-31

Procedure similar to that described for dimethylethylphosphine gold(I) chloride I-27 starting from 1-methylphosphinaneborane I-29 (130 mg, 1.0 mmol) to provide the title compound as an off-white solid (120 mg, 0.35 mmol, 35%). ¹H-NMR (400MHz, CDCl₃): δ ppm 2.16-2.05 (2H, m), 1.95-1.64 (7H, m), 1.55 (3H, d, J=10.9 Hz) 1.39 (1H, m). ³¹P-NMR (162 MHz, CDCl₃): δ ppm -1.38 (s).

4-Methyl-[1,4]oxaphosphinane gold(I) chloride I-34

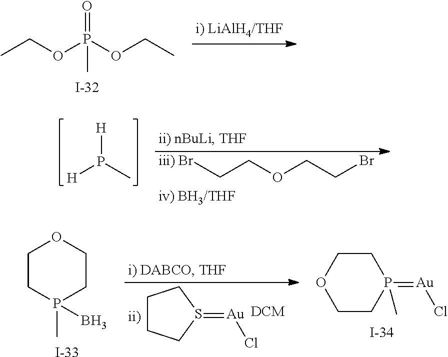

(a) 4-Methyl-[1,4]oxaphosphinaneborane I-33

To a solution of diethyl methylphosphonate (1.5 g, 10.0 mmol) in dry THF (30 mL) was added lithium aluminium hydride (1M in THF, 15 mL, 15.0 mmol) at 0° C., and the mixture allowed to warm to rt and stirred for 4 h. The reaction mixture was cooled to 0° C. whereupon BuLi (1.6 M in hexanes, 12.5 mL, 20 mmol) was added over 5 min and stirring continued at 0° C. for 45 min. 1-Bromo-2-(2-bromoethoxy)ethane (2.3 g, 10 mmol) was then added in one portion and the reaction mixture stirred for further 4 h. Borane-THF complex (1M in THF, 20 mL, 20 mmol) was added and the reaction mixture stirred at rt for an additional 72 h before being diluted with water (60 mL) and 2M HCl (aq., 160 mL) with vigorous stirring. The aqueous phase was extracted with DCM and the combined organic extracts dried over MgSO₄. Concentration in vacuo gave the crude product which was purified by flash column chromatography (Biotage SP1, 25 g KP-Sil column eluting with isohexane to EtOAc) to provide the title compound as a colourless oil (220 mg, 1.7 mmol, 17%).

(b) 4-Methyl-[1,4]oxaphosphinane gold(I) chloride I-34

Procedure similar to that described for dimethylethylphosphine gold(I) chloride I-27 starting from 4-methyl-[1,4]oxaphosphinaneborane I-33 (220 mg, 1.7 mmol) to provide the title compound as an off-white solid (186 mg, 0.5 mmol, 32%). ¹H NMR (400 MHz, CDCl₃): δ ppm 4.19-3.95 (4H, m), 2.24-2.13 (2H, m), 2.09-2.01 (2H, m), 1.75 (3H, d, J=11.1 Hz). ³¹P-NMR (162 MHz, CDCl₃): δ ppm −7.26 (s),

Diethylmethylphosphine gold(I) chloride I-36

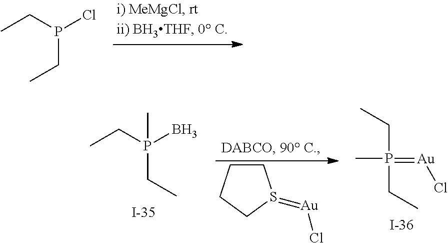

(a) Diethylmethylphosphine borane I-35

To a cold (0) solution of diethylchlorophosphine (1.0 g, 8.0 mmol) in THF (20 mL) under inert atmosphere was slowly added methylmagnesium chloride (3M in THF, 2.7 mL, 8.0 mmol). After warming to rt and being stirred for 4 h, the reaction was cooled to 0° C. prior to the addition of borane-THF complex (1 M in THF, 8 mL, 8.0 mmol). The reaction mixture was allowed to warm up to rt O/N, then was diluted with Et₂O (30 mL) and water (20 mL). The phases were separated and the organic layer was washed with water (2×10 mL) and brine (10 mL) before being dried over MgSO₄ and concentrated in vacuo to provide the title compound as a colourless oil (388 mg, 3.2 mmol, 40%).

(b) Diethylmethylphosphine gold(I) chloride I-36

Procedure similar to that described for dimethylethylphosphine gold(I) chloride I-27 starting from diethylmethylphosphine borane I-35 (385 mg, 3.2 mmol) to provide the title compound as a white solid (475 mg, 1.41 mmol, 44%). ¹H NMR (400 MHz, CDCl₃): δ ppm 1.95-1.75 (4H, m), 1.52 (3H, d, J=10.6 Hz), 1.21 (6H, dt, J=19.7, 7.6 Hz), 1.75 (3H, d, J=11.1 Hz). ³¹P-NMR (162 MHz, CDCl₃): δ ppm 18.13 (s).

1,4-Dimethyl-[1,4]azaphosphinane gold(I) chloride I-39

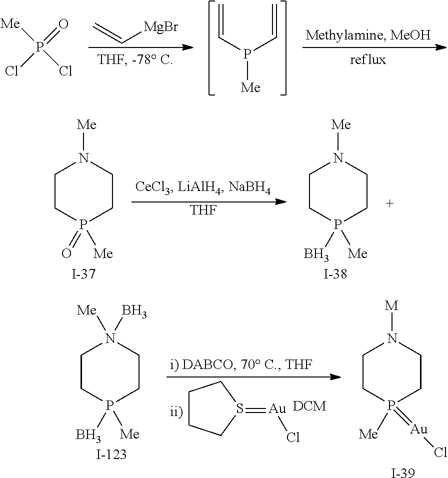

(a) 1,4-Dimethyl-[1,4]azaphosphinane 4-oxide I-37

A solution of methylphosphonic dichloride (2.0 g, 15 mmol) in THF (30 mL) was cooled to −78° C. A solution of vinylmagnesium bromide (1M in THF, 30 mL, 30.0 mmol) was added dropwise, and the resulting mixture was allowed to slowly warm up to rt O/N. The reaction mixture was then transferred to a sealed tube, into which methylamine (2M in THF, 9 mL,18 mmol) was added, followed by MeOH (30 mL). The tube was sealed and heated at 70° C. O/N, after which time the reaction was cooled to rt, and the solvent removed under reduced pressure. The residue was dissolved in a minimum amount of water/MeOH, loaded onto a SCX-2 cartridge (Biotage), washed with water, MeOH and finally NH₃/MeOH solution (2M). Evaporation of the solvent under reduced pressure afforded the title compound as a pale yellow crystalline solid (620 mg, 4.2 mmol, 28%).

(b) 1,4-Dimethyl-[1,4]azaphosphinane borane I-38

Procedure similar to that described for dimethylphosphine borane I-25 starting from 1,4-dimethyl-[1,4]azaphosphinane 4-oxide I-37 (300 mg, 2.0 mmol) to provide the title compound as a ˜1:1 mixture (¹H NMR) with the bis-borane complex I-123 as a white solid (130 mg).

(c) 1,4-Dimethyl-[1,4]azaphosphinane gold(I) chloride I-39

Procedure similar to that described for dimethylethylphosphine gold chloride I-27 starting from the 1:1 mixture of 1,4-dimethyl-[1,4]azaphosphinane borane I-38 and, 1,4-dimethyl-[1,4]azaphosphinane diborane I-123 (130 mg) to provide the title compound as a brown solid (170 mg, 0.46 mmol, 23% over 2 steps. ¹H-NMR (400MHz, CDCl₃): δ ppm 2.90-2.68 (4H, m), 2.35 (3H, s), 2.22-2.15 (2H, m), 2.10-1.98 (2H, m), 1.65 (3H, d, J=11.1 Hz). ³¹P-NMR (162 MHz, CDCl₃): δ ppm −7.30 (s).

[Hydroxymethyl(methyl)phosphanyl]methanol I-40

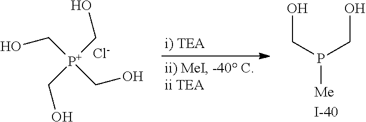

[Hydroxymethyl(methyl)phosphanyl]methanol I-40

A solution of tetrakis(hydroxymethyl)phosphonium chloride (aq., 80%, 7.5 mL, 50 mmol) was concentrated in vacuo to remove the water before adding TEA (30 mL). The resulting mixture was stirred at rt for 18 h. After filtration to remove salts, the supernatant was dissolved in THF (60 mL) and cooled to −40° C. before adding Mel (1.8 mL, 29 mmol). The solution was allowed to warm up to rt O/N and concentrated in vacuo before adding TEA (20 mL). concentration in vacuo provided the title compound as a yellow oil (1.77 g, 16.38 mmol, 33%) which was used straight away in the next step.

4-Methyl-[1,4]sulfonylphosphinane gold(I) chloride I-44

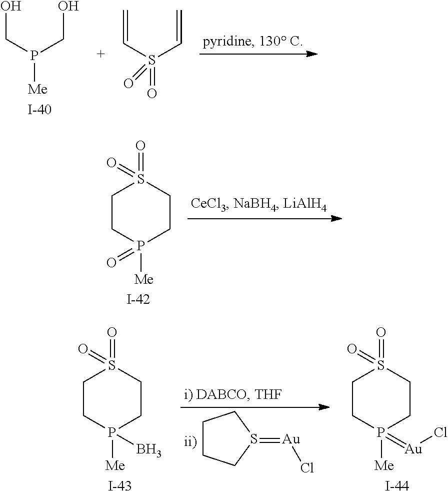

(a) 4-Methyl-[1,4] sulfonylphosphine oxide I-42

To a solution of [hydroxymethyl(methyl)phosphanyl]methanol I-40 (1.7 g, 15.6 mmol) in pyridine (40 mL) was added divinylsulfone (1.7 mL, 16 mmol). The resulting mixture was heated at 130° C. for 5 h. After cooling to rt, MeOH was added to quench the excess of divinylsulfone and the resulting mixture stirred at rt O/N. After concentration in vacuo, acetone (20 mL) was added and the resulting suspension was stirred at rt O/N. The resulting precipitate was collected by filtration and washed with acetone (2×10 mL) to provide the title compound as a beige solid (1.3 g, 7.1 mmol, 46%).

(b) 4-Methyl-[1,4]sulfonylphosphinaneborane I-43

Cerium (Ill) chloride (1.63 g, 6.6 mmol) was suspended in THF (30 mL) and stirred at rt for 30 min. Sodium borohydride (250 mg, 6.6 mmol) was then added and the suspension stirred at rt for a further 30 min. The reaction was cooled to 0° C. at which point 4-methyl-[1,4]sulfonylphosphine oxide I-42 (400 mg, 2.2 mmol) in THF (30 mL) was added dropwise followed by lithium aluminium hydride (1 M in THF, 2.65 mL, 2.65 mmol) also dropwise. The reaction was allowed to warm to rt O/N before being cooled to 0° C. and quenched with a 10% aq. Rochelle salt solution (20 mL). The aqueous layer was extracted with EtOAc (3×10 mL)and the organics combined, washed with brine and dried with Na₂SO₄. Concentration of the filtrate in vacuo provided the title compound as a white solid (140 mg, 0.76 mmol, 35%).

(c) 4-Methyl-[1,4]sulfonylphosphinane gold(I) chloride I-44

Prepared according to the procedure described for dimethylethylphosphine gold(I) chloride I-27 starting from 4-methyl-[1,4]sulfonylphosphinaneborane I-43 (135 mg, 0.7 mmol) to provide, after column chromatography (Biotage Isolera 4, 10 g KP-Sil) eluting with DCM to 5% MeOH in DCM, the title compound as a white solid (31 mg, 0.1 mmol, 11%). ¹H-NMR (400MHz, DMSO-d6): δ ppm 3.60-3.25 (4H, m), 2.63-2.55 (4H, m), 1.82 (3H, d, J=11.9 Hz). ³¹P-NMR (162 MHz, DMSO-d6): δ ppm −4.35 (s).

Dimethyl[2-(methyl)thiazole]phosphine gold(I) chloride I-49 and dimethyl[2-(methyl) oxazole]phosphine gold(I) chloride I-50

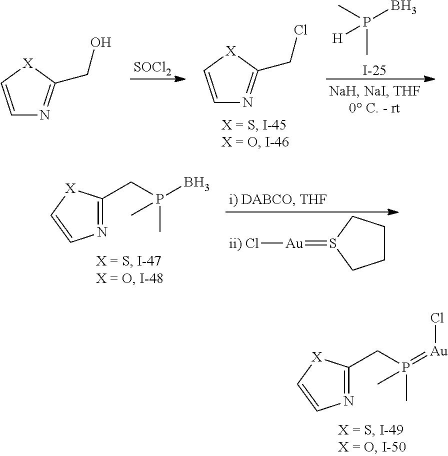

(a) 2-(Chloromethyl)thiazole I-45

To a solution of 2-(hydroxymethyl)thiazole (500 mg, 4.3 mmol) in DCM (25 mL) at 0° C. was added dropwise thionyl chloride (4.4 mL, 60.8 mmol). After stirring for 5 h, the solution was concentrated in vacuo. The resulting solid was triturated with Et₂O (20 mL×2) to provide the title compound as a yellow solid (550 mg, 4.1 mmol, 95%).

(b) 2-(Chloromethyl)oxazole I-46

To a solution of 2-(hydroxymethyl)oxazole (450 mg, 4.5 mmol) in DCM (25 mL) at 0° C. was added dropwise thionyl chloride (3.25 mL, 45 mmol). After stirring for 1 h, water (50 mL) and EtOAc (60 mL) were added. The phases were separated and the organic extracts Concentrated in vacuo to provide the title compound as a white solid (200 mg, 1.7 mmol, 37%).

(c) Dimethyl[2-(methyl)thiazole]phosphine borane I-47

To a solution of dimethylphosphine borane I-25 (200 mg, 2.6 mmol) in THF (30 mL) at 0° C. was added NaH (60% dispersion in mineral oil, 112 mg, 2.8 mmol) in one portion whereupon effervescence was observed. The opaque reaction was stirred at rt for 10 min then cooled back to 0° C. whereupon 2-(chloromethyl)thiazole I-45 (341 mg, 2.6 mmol) and Nal (383 mg, 2.6 mmol) were added. The mixture was allowed to warm to rt O/N, then water (10 mL) and DCM (10 mL) were added and the phases separated. The aqueous phase was extracted with DCM (2×15 mL) and the combined organic extracts washed with brine (20 mL) before passing through a phase separator cartridge (Biotage). Concentration in vacuo gave the crude product which was purified by column chromatography (Biotage Isolera 4, KP-Sil 25 g) eluting with DCM to 3% MeOH in DCM to provide the title compound as a yellow oil (73 mg, 0.4 mmol, 16%).

(d) Dimethyl[2-(methyl)oxazole]phosphine borane I-48

Procedure similar to that described for dimethyl[2-(methyl)thiazole]phosphine borane I-47 starting from 2-(chloromethyl)oxazole I-46 (200 mg, 1.8 mmol). Purification by column chromatography (Biotage Isolera 4, 10 g KP-Sil) eluting with isohexane to 50% EtOAc/isohexane provided the title compound as a colourless oil (183 mg, 1.1 mmol, 64%).

(e) Dimethyl[2-(methyl)thiazole]phosphine gold(I) chloride I-49

Procedure similar to that described for dimethylethylphosphine gold(I) chloride I-27 starting from dimethyl[2-(methyl)thiazole]phosphine borane I-47 (73 mg, 0.41 mmol) to provide the title compound as an off-white solid (17 mg, 0.04 mmol, 11%). ¹H-NMR (400MHz, CDCl₃): δ ppm 7.76 (1H, d, J=3.3 Hz), 7.37 (1H, dd, J=3.3, 1.2 Hz), 3.73 (2H, d, J=11.4 Hz), 1.70 (6H, d, J=10.6 Hz). ³¹P-NMR (162 MHz, CDCl₃): δ ppm 4.52 (s).

(t) Dimethyl[2-(methyl)oxazole]phosphine gold(I) chloride I-50

Procedure similar to that described for dimethylethylphosphine gold(I) chloride I-27 starting from dimethyl[2-(methyl)oxazole]phosphine borane I-48 (180 mg, 1.1 mmol) to provide the title compound as a white solid (165 mg, 0.4 mmol, 39%). ¹H-NMR δ ppm (400MHz, CDCl₃): 7.67 (1H, s), 7.11 (1H, s), 3.45 (2H, d, J=10.6 Hz), 1.69 (6H, d, J=10.6 Hz). ³¹P-NMR (162 MHz, CDCl₃): δ ppm 0.29 (s).

Dimethylcyclopentylphosphine gold(I) chloride I-52

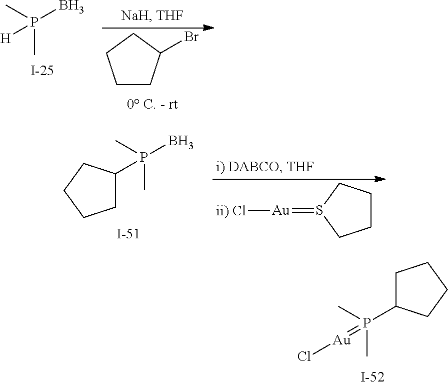

(a) Dimethylcyclopentylphosphine borane I-51

Procedure similar to that described for dimethylethylphosphine borane I-26 starting from dimethylphosphine borane I-25 (200 mg, 2.6 mmol) and bromocyclopentane (0.36 mL, 2.9 mmol) to provide the title compound as a colourless oil (208 mg, 1.4 mmol, 56%).

(b) Dimethylcyclopentylphosphine gold(I) chloride I-52

Procedure similar to that described for dimethylethylphosphine gold(I) chloride I-27 starting from dimethylcyclopentylphosphine borane I-53 (104 mg, 0.72 mmol) to provide the title compound as a colourless oil (58 mg, 0.16 mmol, 22%). ¹H-NMR (400 MHz, CDCl₃): δ ppm 2.11-1.91 (3H, m), 1.85-1.74 (2H, m), 1.72-1.58 (4H, m), 1.56 (6H, d, J=10.6 Hz). ³¹P-NMR (162 MHz, CDCl₃): δ ppm 14.10 (s).

Tert-butyldimethylphosphine gold(I) chloride I-54

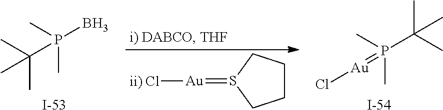

Procedure similar to that described for dimethylethylphosphine gold(I) chloride I-27, starting from tert-butyldimethylphosphine borane I-53 (100 mg, 0.76 mmol) to provide the title compound as a white solid (73 mg, 0.21 mmol, 27%). ¹H-NMR (400 MHz, CDCl₃): δ ppm 1.51 (6H, d, J=10.1 Hz), 1.21 (9H, d, J=16.7 Hz). ³¹P-NMR (162 MHz, CDCl₃): δ ppm 24.61 (s).

Mercapto-N,N-dialkyl-benzamides I-71 to I-81

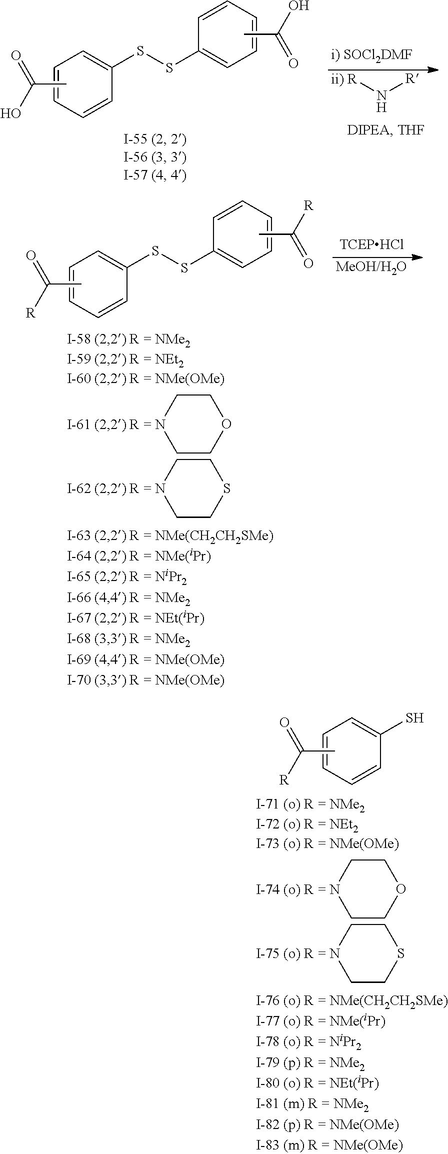

(a) 2,2′-Disulfanediylbis(N,N-dimethylbenzamide) I-58

2,2′-Dithiobenzoic acid I-55 (500 mg, 1.6 mmol) was suspended in anhydrous toluene (5 mL) and DMF (31 μL). Thionyl chloride (310 μL, 4.3 mmol) was added and the reaction mixture stirred at 90° C. for 16 h. Dimethylamine hydrochloride (1.3 g, 16.3 mmol), DIPEA (5.7 mL, 32.6 mmol) and THF (10 mL) were then added and stirred at rt O/N. The reaction mixture was evaporated to dryness, suspended in DCM and washed sequentially with water, 10% aqueous K₂CO₃ and saturated aqueous citric acid. The organic layer was passed through a phase separator cartridge (Biotage) and concentrated in vacuo. The residue was purified by column chromatography (Biotage, Isolera 4, 25 g KP-Sil, eluting with EtOAc) to afford the title compound as a yellow solid (360 mg, 1.0 mmol, 62%).

The following dithiobenzamides I-59 to I-70 were prepared according to the procedure described for 2,2′-disulfanediylbis(N,N-dimethylbenzamide) I-58. All reactions performed using 10 equivalents of the appropriate amine unless otherwise stated.

(b) 2,2′-DisulfanediyIbis(N,N-diethylbenzamide) I-59

Using diethylamine (1.7 mL, 16.3 mmol) the title compound was provided as a yellow oil (164 mg, 0.4 mmol, 48%).

(c) 2,2 ′-Disulfanediylbis(N-methoxy-N-methylbenzamide) I-60

Using N,O-dimethylhydroxylamine hydrochloride (796 mg, 8.2 mmol) the title compound was provided as a colourless gum (106 mg, 0.3 mmol, 33%).

(d) (Disulfanediylbis(4,1-phenylene))bis(morpholinomethanone) I-61

Using morpholine (0.71 mL, 8.2 mmol) the title compound was provided as a yellow gum (203 mg, 0.5 mmol, 56%).

(e) (DisulfanediyIbis(4,1-phenylene))bis(thiomorpholinomethanone) I-62

Using thiomorpholine (0.82 mL, 8.2 mmol) the title compound was provided as an off-white solid (140 mg, 0.3 mmol, 36%).

(t) 2,2′-DisulfanediyIbis(N-methyl-N-(2-(methylthio)ethyObenzamide) I-63

Procedure similar to that described for 2,2′-disulfanediylbis(N,N-dimethylbenzamide I-58 except 2,2′-dithiobenzoic acid I-55 (100 mg, 0.33 mmol) and N-methyl-2-(methylthio)ethanamine (100 mg, 0.95 mmol) were used. The title compound was provided as a yellow gum (63 mg, 0.13 mmol, 40%).

(g) 2,2′-DisulfanediyIbis(N-isopropyl-N-methylbenzamide) I-64

Procedure similar to that described for 2,2′-disulfanediylbis(N,N-dimethylbenzamide I-58 except 2,2′-dithiobenzoic acid I-55 (250 mg, 0.82 mmol) and N-isopropylmethylamine (0.51 mL, 4.9 mmol) were used. The title compound was provided as a yellow gum (175 mg, 0.42 mmol, 51%).

(h) 2,2′-DisulfanediyIbis(N,N-diisopropylbenzamide) I-65

Procedure similar to that described for 2,2′-disulfanediylbis(N,N-dimethylbenzamide I-58 except 2,2′-dithiobenzoic acid I-55 (250 mg, 0.8 mmol) and diisopropylamine (0.69 mL, 4.9 mmol) were used. The title compound was provided as a pale yellow solid (144 mg, 0.3 mmol, 37%).

(i) 4,4′-DisulfanediyIbis(N,N-dimethylbenzamide) I-66

Procedure similar to that described for 2,2′-disulfanediylbis(N,N-dimethylbenzamide I-58 except 4,4′-dithiobenzoic acid I-57 (250 mg, 0.82 mmol) and dimethylamine hydrochloride (665 mg, 8.2 mmol) were used. The title compound was provided as a white solid (132 mg, 0.37 mmol, 45%).

W 2,2′-DisulfanediyIbis(N-ethyl-N-isopropylbenzamide) I-67

Procedure similar to that described for 2,2′-disulfanediylbis(N,N-dimethylbenzamide I-58 except 2,2′-dithiobenzoic acid I-55 (250 mg, 0.82 mmol) and N-ethylisopropylamine (711 mg, 8.2 mmol) were used. The title compound was provided as a yellow gum (124 mg, 0.28 mmol, 34%).

(k) 3,3′-DisulfanediyIbis(N,N-dimethylbenzamide) I-68

Procedure similar to that described for 2,2′-disulfanediylbis(N,N-dimethylbenzamide I-58 except 3,3′-dithiobenzoic acid I-56 (250 mg, 0.82 mmol) and dimethylamine hydrochloride (665 mg, 8.2 mmol) were used. The title compound was provided as a colourless gum (125 mg, 0.35 mmol, 42%).

(I) 4,4′-DisulfanediyIbis(N-methoxy-N-methylbenzamide) I-69

Procedure similar to that described for 2,2′-disulfanediylbis(N,N-dimethylbenzamide I-58 except 4,4′-dithiobenzoic acid I-57 (250 mg, 0.82 mmol) and N,O-dimethylhydroxylamine hydrochloride (796 mg, 8.2 mmol) were used. The title compound was provided as a white solid (108 mg, 0.28 mmol, 34%).

(m)3,3′-DisulfanediyIbis(N-methoxy-N-methylbenzamide) I-70

Procedure similar to that described for 2,2′-disulfanediylbis(N,N-dimethylbenzamide I-58 except 3,3′-dithiobenzoic acid I-56 (250 mg, 0.82 mmol) and N,O-dimethylhydroxylamine hydrochloride (796 mg, 8.2 mmol) were used. The title compound was provided as a pale yellow gum (165 mg, 0.42 mmol, 51%).

(n) 2-Mercapto-N,N-dimethyl-benzamide I-71

2,2′-Disulfanediylbis(N,N-dimethylbenzamide) I-58 (50 mg, 0.14 mmol) was dissolved in MeOH (4 mL) and water (3 mL) before adding TCEP.HCl (200 mg, 0.7 mmol) in one portion. The reaction mixture was stirred at rt O/N and the solvent removed under reduced pressure. The residue was dissolved in water and extracted with DCM. The combined organic extracts were passed through a phase separator cartridge (Biotage) and concentrated in vacuo to provide the title compound as a yellow oil (45 mg, 0.25 mmol, 89%).

(o) 2-Mercapto-N,N-diethyl-benzamide I-72

Procedure similar to that described for 2-mercapto-N,N-dimethyl-benzamide I-71 except 2,2′-disulfanediylbis(N,N-diethylbenzamide) I-59 (40 mg, 0.09 mmol) and TCEP.HCl (138 mg, 0.48 mmol) were used. The title compound was provided as a colourless gum (28 mg, 0.13 mmol, 70%).

(p) 2-Mercapto-N-methoxy-N-methyl-benzamide I-73

Procedure similar to that described for 2-mercapto-N,N-dimethyl-benzamide I-71 except 2,2′-disulfanediylbis(N-methoxy-N-methylbenzamide) I-60 (50 mg, 0.13 mmol) and TCEP.HCl (183 mg, 0.64 mmol) were used. The title compound was provided as a colourless gum (46 mg, 0.23 mmol, 92%).

(q) (2-Mercapto-phenyl)-morpholin-4-yl-methanone I-74

Procedure similar to that described for 2-mercapto-N,N-dimethyl-benzamide I-71 except (disulfanediyIbis(4,1-phenylene))bis(morpholinomethanone) I-61 (42 mg, 0.09 mmol) and TCEP.HCl (135 mg, 0.47 mmol) were used. The title compound was provided as a white solid (39 mg, 0.17 mmol, 93%).

(r) (2-Mercapto-phenyl)-thiomorpholin-4-yl-methanone I-75

Procedure similar to that described for 2-mercapto-N,N-dimethyl-benzamide I-71 except (disulfanediyIbis(4,1-phenylene))bis(thiomorpholinomethanone) I-62 (50 mg, 0.11 mmol) and TCEP.HCl (150 mg, 0.53 mmol) were used. The title compound was provided as a yellow solid (46 mg, 0.19 mmol, 91%)

(s) 2-Mercapto-N-(2-methylsulfanyl-ethyl)-benzamide I-76

Procedure similar to that described for 2-mercapto-N,N-dimethyl-benzamide I-71 except 2,2′-disulfanediylbis(N-methyl-N-(2-(methylthio)ethyl)benzamide) I-63 (29 mg, 0.06 mmol) and TCEP.HCl (86 mg, 0.3 mmol) were used. The title compound was provided as a yellow gum (29 mg, 0.12 mmol, quantitative).

(t) N-Isopropyl-2-mercapto-N-methyl-benzamide I-77

Procedure similar to that described for 2-mercapto-N,N-dimethyl-benzamide I-71 except 2,2′-disulfanediylbis(N-isopropyl-N-methylbenzamide) I-64 (43 mg, 0.10 mmol) and TCEP.HCl (148 mg, 0.52 mmol) were used. The title compound was provided as a yellow oil (42 mg, 0.2 mmol, 97%).

(u) N,N-Diisopropyl-2-mercapto-benzamide I-78

Procedure similar to that described for 2-mercapto-N,N-dimethyl-benzamide I-71 except 2,2′-disulfanediyIbis(N,N-diisopropylbenzamide) I-65 (44 mg, 0.09 mmol) and TCEP.HCl (135 mg, 0.47 mmol) were used. The title compound was provided as a white solid (42 mg, 0.18 mmol, 94%).

(v) 4-Mercapto-N,N-dimethyl-benzamide I-79

Procedure similar to that described for 2-mercapto-N,N-dimethyl-benzamide I-71 except 4,4′-disulfanediylbis(N,N-dimethylbenzamide) I-66 (47 mg, 0.13 mmol) and TCEP.HCl (188 mg, 0.66 mmol) were used. The title compound was provided as a colourless oil (50 mg, 0.28 mmol, quantitative).

(w) N-Ethyl-N-isopropyl-2-mercapto-benzamide I-80

Procedure similar to that described for 2-mercapto-N,N-dimethyl-benzamide I-71 except 2,2′-disulfanediylbis(N-ethyl-N-isopropylbenzamide) I-67 (42 mg, 0.09 mmol) and TCEP.HCl (135 mg, 0.47 mmol) were used. The title compound was provided as a pale yellow oil (38 mg, 0.17 mmol, 90%).

(x) 3-Mercapto-N,N-dimethyl-benzamide I-81

Procedure similar to that described for 2-mercapto-N,N-dimethyl-benzamide I-71 except 3,3′-disulfanediylbis(N,N-dimethylbenzamide) I-68 (42 mg, 0.12 mmol) and TCEP.HCl (168 mg, 0.59 mmol) were used. The title compound was provided as a pale yellow oil (43 mg, 0.24 mmol, quantitative).

(y) 4-Mercapto-N-methoxy-N-methyl-benzamide I-82

Procedure similar to that described for 2-mercapto-N,N-dimethyl-benzamide I-71 except 4,4′-disulfanediylbis(N-methoxy-N-methylbenzamide) I-69 (63 mg, 0.16 mmol) and TCEP.HCl (230 mg, 0.8 mmol) were used. The title compound was provided as a colourless oil (56 mg, 0.28 mmol, 88%).

(z) 3-Mercapto-N-methoxy-N-methyl-benzamide I-83

Procedure similar to that described for 2-mercapto-N,N-dimethyl-benzamide I-71 except 3,3′-disulfanediylbis(N-methoxy-N-methylbenzamide) I-70 (46 mg, 0.12 mmol) and TCEP.HCl (168 mg, 0.59 mmol) were used. The title compound was provided as a colourless oil (53 mg, 0.27 mmol, quantitative).

5-(2-Methoxycarbonyl-ethylsulfanyl)-pyrimidine-4-carboxylic acid methyl ester I-86

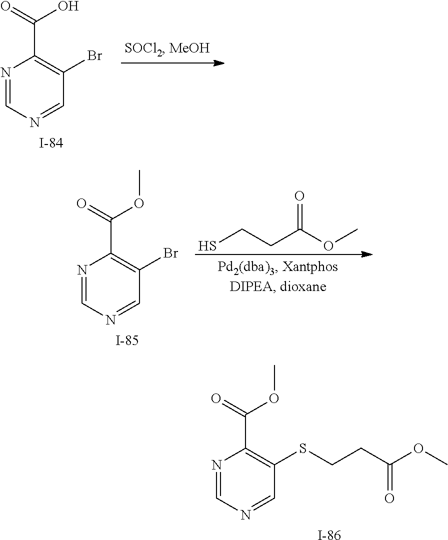

(a) 5-Bromo-pyrimidine-4-carboxylic acid methyl ester I-85

5-Bromo-4-pyrimidine carboxylic acid I-84 (858 mg, 4.23 mmol) was dissolved in MeOH (15 mL) and thionyl chloride (77 μL, 1.06 mmol) added dropwise at rt. The reaction mixture was heated to 70° C. and stirred at this temperature for 3 h. The reaction mixture was then cooled to rt and evaporated to dryness. The residue was re-dissolved in a mixture of water (25 mL) and saturated aq. NaHCO₃ (25 mL) before extracting with EtOAc (3×50 mL). The combined organic extracts were then washed with saturated aqueous NaHCO₃ (40 mL) and brine (40 mL) before drying over MgSO₄. Concentration in vacuo provided the title compound as a brown solid (502 mg, 2.31 mmol, 55%).

(b) 5-(2-Methoxycarbonyl-ethylsulfanyl)-pyrimidine-4-carboxylic acid methyl ester I-86

A mixture of 5-bromo-pyrimidine-4-carboxylic acid methyl ester I-85, (500 mg, 2.3 mmol), methyl-3-mercaptopropionate (280 uL, 2.3 mmol), Pd₂(dba)₃ (84 mg, 0.092 mmol), Xantphos (106 mg, 0.18 mmol), DIPEA (801 uL, 4.6 mmol) and dioxane (15 mL) was degassed with nitrogen and the mixture heated at 110° C. until LC-MS (AnalpH2_MeOH_4min) indicated completion of the reaction. The reaction mixture was concentrated in vacuo and the residue diluted with EtOAc (100 mL) before being washed with saturated aqueous NH₄Cl (30 mL), saturated aqueous NaHCO₃ (30 mL) and brine (30 mL). The organic phase was dried over MgSO₄ before being concentrated in vacuo. The residue was purified by column chromatography (Biotage, Isolera 4, 100 g KP-Sil, eluting with 20% EtOAc/isohexane to EtOAc) to afford the title compound as an off-white solid (378 mg, 1.5 mmol, 64%).

Sulfanyl-propionic acid methyl esters I-94 to I-98

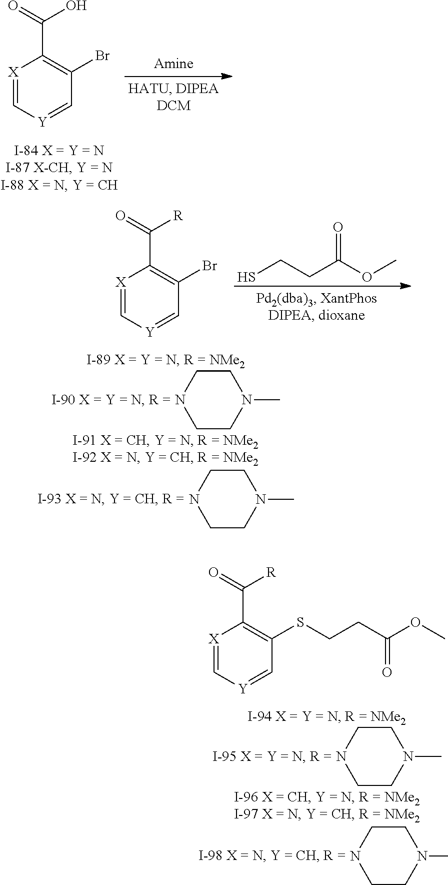

(a) 5-Bromo-pyrimidine-4-carboxylic acid dimethylamide I-89

5-Bromo-4-pyrimidine carboxylic acid I-84 (410 mg, 2.0 mmol) and dimethylamine hydrochloride (329 mg, 4.0 mmol) were combined and suspended in DCM (13 mL). DIPEA (1.1 mL, 6.1 mmol) was added followed by HATU (1.1 g, 2.9 mmol) and the reaction stirred at rt O/N. The reaction was diluted with DCM and washed with water and the layers separated. The aqueous fraction was extracted with DCM (×2) and the combined organic extracts passed through a phase separator cartridge (Biotage) and concentrated in vacuo. The residue was purified by column chromatography (Biotage, Isolera 4, 50 g KP-Sil, eluting with 50% EtOAc/isohexane to EtOAc) to afford the title compound as a pale yellow oil (353 mg, 1.5 mmol, 76%).

(b) (5-Bromo-pyrimidin-4-yl)-(4-methyl-piperazin-1-yl)-methanone I-90

Procedure similar to that described for 5-bromo-pyrimidine-4-carboxylic acid dimethylamide I-89 except 1-methylpiperazine (546 82 L, 4.9 mmol) was used. No final purification was performed. The crude title compound was provided as a yellow oil (1.9 g, 6.7 mmol, >100%).

(c) 3-Bromo-N,N-dimethyl-isonicotinamide I-91

Procedure similar to that described for 5-bromo-pyrimidine-4-carboxylic acid dimethylamide I-89 except 3-bromoisonicotinic acid I-87 (350 mg, 1.7 mmol) and dimethylamine hydrochloride (141 mg, 1.7 mmol) were used. The title compound was provided as an orange oil (1.1 g, 6.7 mmol, >100%).

(d) 3-Bromo-pyridine-2-carboxylic acid dimethylamide I-92

Procedure similar to that described for 5-bromo-pyrimidine-4-carboxylic acid dimethylamide I-89 except 3-bromopyridine-2-carboxylic acid I-88 (350 mg, 1.7 mmol) and dimethylamine hydrochloride (141 mg, 1.7 mmol) were used. The crude title compound was provided as an orange oil (1.2 g, 6.7 mmol, >100%).

(e) (3-Bromo-pyridin-2-yl)-(4-methyl-piperazin-1-yl)-methanone I-93

Procedure similar to that described for 5-bromo-pyrimidine-4-carboxylic acid dimethylamide I-89 except 3-bromopyridine-2-carboxylic acid I-88 (400 mg, 2.0 mmol) and 1-methylpiperazine (0.27 mL, 2.4 mmol) were used. The title compound was provided as a colourless oil (520 mg, 1.8 mmol, 92%).

(t) 3-(4-Dimethylcarbamoyl-pyrimidin-5-ylsulfanyl)-propionic acid methyl ester I-94

Procedure similar to that described for 5-(2-methoxycarbonyl-ethylsulfanyl)-pyrimidine-4-carboxylic acid methyl ester I-86 except 5-bromo-pyrimidine-4-carboxylic acid dimethylamide I-89 (353 mg, 1.5 mmol) was used. The title compound was provided as a yellow oil (130 mg, 0.48 mmol, 31%).

(g) 3-[4-(4-Methyl-piperazine-1-carbonyl)-pyrimidin-5-ylsulfanyl]-propionic acid methyl ester I-95

Procedure similar to that described for 5-(2-methoxycarbonyl-ethylsulfanyl)-pyrimidine-4-carboxylic acid methyl ester I-86 except (5-bromo-pyrimidin-4-yl)-(4-methyl-piperazin-1-yl)-methanone I-90 (700 mg, 2.5 mmol) was used. Purification was carried out by preparative HPLC (basic conditions) to provide the title compound as a colourless oil (85 mg, 0.3 mmol, 11%).

(h) 3-(4-Dimethylcarbamoyl-pyridin-3-ylsulfanyl)-propionic acid methyl ester I-96

Procedure similar to that described for 5-(2-methoxycarbonyl-ethylsulfanyl)-pyrimidine-4-carboxylic acid methyl ester I-86 except 3-bromo-N,N-dimethyl-isonicotinamide I-91 (396 mg, 1.7 mmol) was used. Purification by reverse phase column chromatography (Biotage, Isolera 4, 120 g KP-C18-HS, eluting with water to MeOH) afforded the title compound as a yellow oil (185 mg, 079 mmol, 41%).

(i) 3-(2-Dimethylcarbamoyl-pyridin-3-ylsulfanyl)-propionic acid methyl ester I-97

Procedure similar to that described for 5-(2-methoxycarbonyl-ethylsulfanyl)-pyrimidine-4-carboxylic acid methyl ester I-86 except 3-bromo-pyridine-2-carboxylic acid dimethylamide I-92 (396 mg, 1.7 mmol) was used. Purification by reverse phase column chromatography (Biotage, Isolera 4, 120 g KP-C18-HS, eluting with water to MeOH) afforded the title compound as a yellow oil (145 mg, 0.5 mmol, 32%).

(j) 3-[2-(4-Methyl-piperazine-1-carbonyl)-pyridin-3-ylsulfanyl]-propionic acid methyl ester I-98

Procedure similar to that described for 5-(2-methoxycarbonyl-ethylsulfanyl)-pyrimidine-4-carboxylic acid methyl ester I-86 except (3-bromo-pyridin-2-yl)-(4-methyl-piperazin-1-yl)-methanone I-93 (520 mg, 1.8 mmol) was used. Purification was carried out by preparative HPLC (basic conditions) to provide the title compound as a white solid (157 mg, 0.5 mmol, 27%).

3-(Pyrimidin-5-ylsulfanyl)-propionic acid methyl ester I-101 and 3-(2-methyl-pyrimidin-5-ylsulfanyl)-propionic acid methyl ester I-102

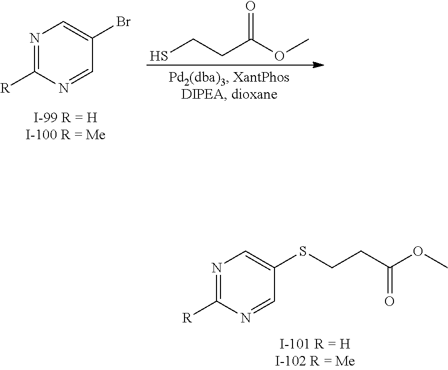

3-(Pyrimidin-5-ylsulfanyl)-propionic acid methyl ester I-101

Procedure similar to that described for 5-(2-methoxycarbonyl-ethylsulfanyl)-pyrimidine-4-carboxylic acid methyl ester I-86 except 5-bromopyrimidine I-99 (300 mg, 1.9 mmol) was used. The title compound was provided as a yellow oil (231 mg, 1.2 mmol, 62%).

In a slight modification to the above procedure, purification by preparative HPLC (acidic conditions) also provided the title compound as a colourless oil (1.2 g, 5.8 mmol, 92%).

3-(2-Methyl-pyrimidin-5-ylsulfanyl)-propionic acid methyl ester 1-102

Procedure similar to that described for 5-(2-methoxycarbonyl-ethylsulfanyl)-pyrimidine-4-carboxylic acid methyl ester I-86 except 5-bromo-2-methylpyrimidine I-100 (300 mg, 1.7 mmol) was used. The title compound was provided as a colourless oil (101 mg, 0.48 mmol, 27%).

Thioacetic acid 2, 6-dimethyl-tetrahydro-pyran-4-yl ester I-106

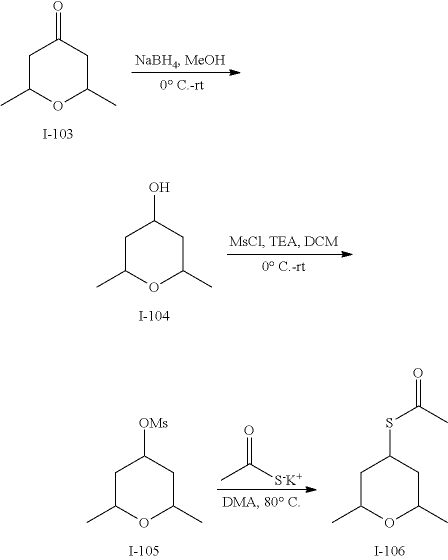

(a) 2, 6-Dimethyl-tetrahydro-pyran-4-ol I-104

2,6-Dimethyltetrahydro-4H-pyran-4-one (as a mixture of diastereoisomers) I-103 (360 mg, 2.77 mmol) was dissolved in anhydrous MeOH (10 mL) and sodium borohydride (116 mg, 2.77 mmol) added portion-wise at 0° C. The reaction mixture was allowed to warm to rt over the course of 18 h whereupon the reaction was quenched with saturated aq. ammonium chloride. The aqueous phase was extracted with Et₂O (×2) and the combined organic extracts washed with brine before passing through a phase separator cartridge (Biotage). Concentration in vacuo provided the title compound as a mixture of diastereoisomers (242 mg) which was used without further purification.

(b) Methanesulfonic acid 2, 6-dimethyl-tetrahydro-pyran-4-yl ester I-105

To a cooled (0° C.) solution of 2,6-dimethyl-tetrahydro-pyran-4-ol I-104 (242 mg, 1.9 mmol) in anhydrous DCM (10 mL) was added mesyl chloride (0.17 mL, 2.2 mmol) followed by TEA (0.51 mL, 3.7 mmol). The reaction mixture was stirred at 0° C. for 4 h whereupon water was added and the aqueous layer extracted with DCM (×2). The combined organic extracts were washed with saturated sodium bicarbonate and brine before passing through a phase separator (Biotage). Concentration in vacuo provided the crude title compound as a mixture of diastereoisomers as a yellow oil (490 mg).

(c) Thioacetic acid 2, 6-dimethyl-tetrahydro-pyran-4-yl ester I-106

Methanesulfonic acid 2,6-dimethyl-tetrahydro-pyran-4-yl ester (490 mg, crude) I-105 was dissolved in DMA (7 mL) and potassium thioacetate (640 mg, 5.5 mmol) added in one portion. The reaction mixture was heated at 80° C. for 24 h. The reaction mixture was diluted with water and the aqueous residue extracted with Et₂O (×3). The combined organic extracts were concentrated in vacuo. The residue was purified by column chromatography (Biotage, SP1, 10 g KP-Sil, eluting with isohexane to 20% EtOAc/isohexane) to afford the title compound as a mixture of diastereoisomers (159 mg, 0.85 mmol, 46% over 3 steps).

4-Methyl-tetrahydro-pyran-4-thiol I-110

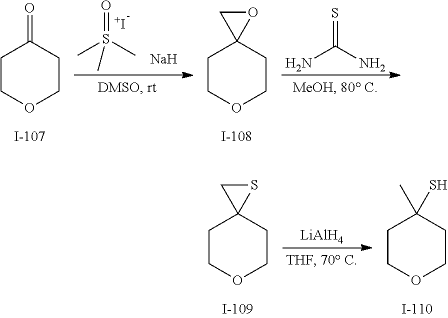

(a) 1,6-Dioxa-spiro[2.5]octane I-108

Trimethylsulfoxonium iodide (286 mg, 13 mmol) was dissolved in DMSO (20 mL) under an atmosphere of nitrogen. NaH (60% dispersion in mineral oil, 520 mg, 13 mmol) was then added portion-wise (NB. vigorous effervescence observed). The resultant suspension was stirred at rt for 1 h whereupon tetrahydro-4H-pyran-4-one I-107 (0.93 mL, 10 mmol) was added dropwise. The reaction mixture was stirred at rt for an additional 1 h where it was then poured into a water/ice slurry. The aqueous phase was extracted with Et₂O (×3) and the combined organic extracts washed with water and brine before drying over MgSO₄. Concentration in vacuo provided the title compound as a pale yellow oil (725 mg, 6.3 mmol, 63%).

(b) 6-Oxa-1-thia-spiro[2.5]octane I-109

1,6-Dioxa-spiro[2.5]octane I-108 (725 mg, 6.3 mmol) was dissolved in anhydrous MeOH (20 mL) and thiourea (480 mg, 6.3 mmol) added. The reaction mixture was heated at 80° C. for 4.5 h at which point water was added. The aqueous phase was extracted with Et₂O (×3) and the combined organic extracts washed with brine before drying over MgSO₄. The residue was purified by column chromatography (Biotage, SP1, 10 g KP-Sil, eluting with isohexane to 20% EtOAc/isohexane) to afford the title compound (130 mg, 1 mmol, 16%).

(c) 4-Methyl-tetrahydro-pyran-4-thiol I-110

6-Oxa-1-thia-spiro[2.5]octane I-109 (130 mg, 1 mmol) was dissolved in THF (3.5 mL) and heated to 70° C. under an atmosphere of nitrogen. Lithium aluminium hydride (1 M in THF, 0.5 mL, 0.5 mmol) was then added and the reaction mixture stirred for 1 h. The reaction mixture was cooled to 0° C. and HCl (1 N, 3.5 mL) added dropwise. The aqueous phase was then extracted with Et₂O (2×10 mL) and the combined organic extracts concentrated in vacuo. Purification by column chromatography (Biotage, SP1, 10 g KP-Sil, eluting with pentane to 10% Et₂O/pentane) afforded the title compound (56 mg, 0.42 mmol, 42%).

(±)Thioacetic acid S-((3S, 4S)-3-methyl-tetrahydro-pyran-4-yl)ester I-113

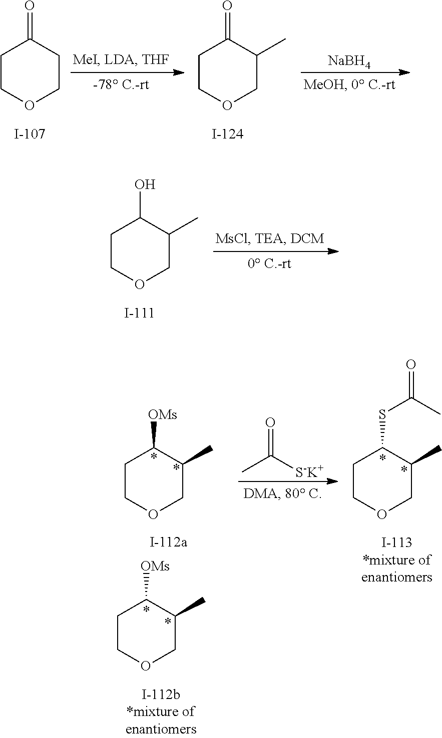

(a) 3-Methyl-tetrahydro-pyran-4-one I-124

Diisopropylamine (1.1 mL, 6.0 mmol) in THF (10 mL) was cooled to −78° C. and n-butyllithium (1.6 M hexanes, 3.8 mL, 6.0 mmol) added dropwise. The reaction mixture was stirred at −78° C. and allowed to gradually warm to rt over the course of 2 h before cooling to −78° C. once again. Tetrahydro-4H-pyran-4-one I-107 (500 mg, 5.0 mmol) as a solution in THF (20 mL) and HMPA (0.88 mL) was then added dropwise and the reaction was subsequently stirred at −78° C. and allowed to gradually warm to rt over the course of 2 h. The reaction mixture was cooled to 0° C. whereupon saturated aqueous ammonium chloride was added and the aqueous phase extracted with Et₂O (×2). Concentration under reduced pressure provided the crude residue which was purified by column chromatography (Biotage, SP1, 25 g KP-Sil, eluting with isohexane to 20% EtOAc/isohexane) to afford the title compound (400 mg, 2.8 mmol, 56%).

(b) 3-Methyl-tetrahydro-pyran-4-ol I-111

Procedure similar to that described for 2,6-dimethyl-tetrahydro-pyran-4-ol I-104 except 3-methyl-tetrahydro-pyran-4-one I-124 (350 mg, 2.5 mmol) was used. The crude title compound was provided as a mixture of diasteroisomers (440 mg).

(a) (±) (3S, 4R)-3-Methyl-tetrahydro-pyran-4-ol I-112a

Procedure similar to that described for methanesulfonic acid 2,6-dimethyl-tetrahydro-pyran-4-yl ester I-105 except 3-methyl-tetrahydro-pyran-4-ol I-111 (140 mg, 1.2 mmol) was used. The title compound was provided (127 mg, 0.65 mmol, 54%). The trans-isomer I-112b was also isolated (30 mg, 0.15 mmol, 13%).

(b) (±) Thioacetic acid S-((3S, 4S)-3-methyl-tetrahydro-pyran-4-yl)ester I-113

Procedure similar to that described for thioacetic acid 2,6-dimethyl-tetrahydro-pyran-4-yl ester I-106 except (±) (3S, 4R)-3-methyl-tetrahydro-pyran-4-ol I-112a (111 mg, 0.57 mmol) was used. The title compound was provided (44 mg, 0.25 mmol, 44%).

4-Acetylsulfanyl-piperidine-1-carboxylic acid methyl ester I-121 and 4-acetylsulfanyl-piperidine-1-carboxylic acid ethyl ester I-122

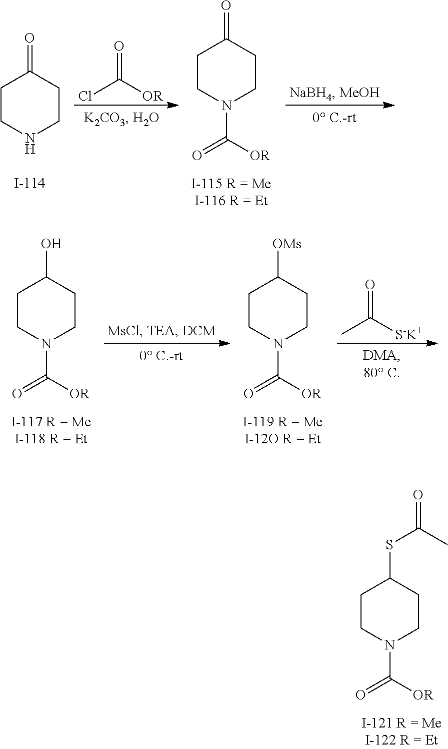

(a) 4-Oxo-piperidine-1-carboxylic acid methyl ester I-115

To a cooled (0° C.) solution of 4-piperidone I-114 (300 mg, 3.0 mmol) in water (2 mL) was added a solution of potassium carbonate (1.05 g, 7.6 mmol) in water (5 mL) followed by methyl chloroformate (350 μl, 4.5 mmol). The reaction mixture was stirred at 0° C. for 3 h. The mixture was diluted with DCM, the layers separated and the aqueous phase extracted with DCM (×3). The combined organic extracts were passed through a phase separator cartridge (Biotage) and concentrated in vacuo. Purification by flash column chromatography (Biotage SP1, 25 g KP-Sil, eluting with isohexane to EtOAc) provided the title compound as a colourless oil (320 mg, 2.0 mmol, 68%).

(b) 4-Hydroxy-piperidine-1-carboxylic acid methyl ester I-117

To a solution of 4-oxo-piperidine-1-carboxylic acid methyl ester I-115 (315 mg, 2.0 mmol) in MeOH (5 mL) at 0° C. was added sodium borohydride (114 mg, 3.0 mmol). The reaction mixture was stirred at 0° C. for 2 h. The reaction was quenched with saturated aqueous ammonium chloride (5 mL), MeOH was removed in vacuo and the aqueous layer was extracted with DCM (×3). The combined organic extracts were passed through a phase separator cartridge (Biotage) and concentrated in vacuo to afford the crude product which was purified by column chromatography (Biotage, SP1, 25 g KP-Sil, eluting with isohexane to EtOAc) to afford title compound as a colourless oil (140 mg, 0.88 mmol, 44%).

(c) 4-Methanesulfonyloxy-piperidine-1-carboxylic acid methyl ester I-119

To a solution of 4-hydroxy-piperidine-1-carboxylic acid methyl ester I-117 (140 mg, 0.88 mmol) in DCM (3 mL) was added mesyl chloride (82 μL, 1.0 mmol) and TEA (245 μL, 1.76 mmol). The reaction mixture was stirred at 0° C. for 1 h. The reaction was quenched with water (5 mL), the layers separated and the aqueous layer extracted with DCM (3×5 mL). The combined organic extract was washed with saturated aqueous sodium bicarbonate (10 mL) and brine (10 mL), passed through a phase separator cartridge (Biotage) and concentrated in vacuo. The crude product was purified by flash column chromatography (Biotage SP1, 10 g KP-Sil, eluting with isohexane to EtOAc) to afford the title compound as a colourless oil (168 mg, 0. 71 mmol, 80%).

(d) 4-Acetylsulfanyl-piperidine-1-carboxylic acid methyl ester I-121

To a solution of 4-methanesulfonyloxy-piperidine-1-carboxylic acid methyl ester I-119 (168 mg, 0.71 mmol) in DMA (4 mL) was added potassium thioacetate (243 mg, 2.1 mmol). The reaction was heated at 80° C. for 18 h. The reaction was cooled to rt and Et₂O (10 mL) and water (10 mL) were added. The layers were separated and the aqueous layer extracted with Et₂O (3×10 mL). The combined organic extract was washed with water (10 mL) and brine (10 mL) before passing through a phase separator cartridge (Biotage). The crude residue was purified by flash column chromatography (Biotage SP1, 25 g KP-Sil, eluting with isohexane to EtOAc) to afford the title compound as a pale orange oil (78 mg, 0.36 mmol, 51%).

(e) 4-Oxo-piperidine-1-carboxylic acid ethyl ester I-116

Procedure similar to that described for 4-oxo-piperidine-1-carboxylic acid methyl ester I-115 except ethyl chloroformate (0.43 mL, 4.5 mmol) was used. The title compound was provided as a colourless oil (332 mg, 1.9 mmol, 65%).

(t) 4-Hydroxy-piperidine-1-carboxylic acid ethyl ester I-118

Procedure similar to that described for 4-hydroxy-piperidine-1-carboxylic acid methyl ester I-117. The title compound was provided as a colourless oil (337 mg, 1.9 mmol, 100%).

(g) 4-Methanesulfonyloxy-piperidine-1-carboxylic acid ethyl ester I-120

Procedure similar to that described for 4-methanesulfonyloxy-piperidine-1-carboxylic acid methyl ester I-119. The title compound was provided as a colourless oil (423 mg, 1.7 mmol, 94%).

(h) 4-Acetylsulfanyl-piperidine-1-carboxylic acid ethyl ester I-122

Procedure similar to that described for 4-acetylsulfanyl-piperidine-1-carboxylic acid methyl ester I-121. The title compound was provided as a pale red oil (218 mg, 0.9 mmol, 59%).

Thioacetic acid S-(tetrahydro-pyran-2-yl) ester I-126

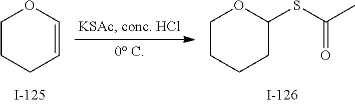

Potassium thioacetate (460 mg, 4.0 mmol) was dissolved in conc. HCl (32%, 10.2M) and cooled to 0° C. Dihydropyran I-125 (0.37 mL, 4.0 mmol) was then added dropwise and the reaction mixture stirred at 0° C. for 2 h whereupon the reaction mixture was concentrated in vacuo. The residue was purified by column chromatography (Biotage, SP1, 10 g KP-Sil, eluting with isohexane to 10% EtOAc/isohexane) to afford the title compound (630 mg, 3.9 mmol, 98%).

### EXAMPLE 1

Compounds of the formula I were synthesised via the coupling of chloro(trialkyl phosphine) gold(I) complexes of formula VII with thiol derivatives of general formula III:

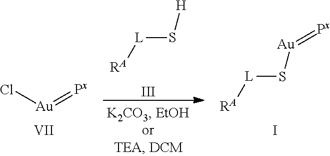

Method A: To a stirred suspension of chlorophosphine gold(I) compound VII (0.32 mmol) in EtOH (1 mL) at 0° C. was slowly added the appropriate thiol III (0.32 mmol) as a solution in aqueous K₂CO₃ (10% w/v, 1 mL). The reaction mixture was then stirred at 0° C. for 1 h before it was diluted with water (5 mL) and extracted with DCM (4×15 mL). The combined organic extracts were passed through a phase separator cartridge (Biotage) and the solvent evaporated to provide the title compound I.

Method B: As Method A, except the thiol III was pre-dissolved in a mixture of K₂CO₃ (aq., 1 mL) and EtOH (1 mL).

Method C: As Method A, except the reaction was heated at 50° C. for 18 h.

Method D: The appropriate thiol III (0.17 mmol) and chlorophosphine gold(I) compound VII (0.17 mmol) were combined and dissolved in DCM (5 mL) under an atmosphere of nitrogen. The solution was cooled to 0° C. before TEA (0.34 mmol) was added dropwise over 5 min. The reaction was stirred at 0° C. for 45 min whereupon the reaction was diluted with water (15 mL) and the layers separated. The aqueous residue was extracted with DCM (2×10 mL) and the combined organic extracts washed with brine (1×15 mL) before passing through a phase separator cartridge (Biotage). Concentration in vacuo afforded the title compound I.

Method E: To a stirred suspension of chlorophosphine gold(I) compound VII (0.32 mmol) in EtOH or MeOH (1 mL) at 0° C. was slowly added the appropriate thiol III (0.32 mmol) as a solution in 10% K₂CO₃ (aq., 1 mL). The reaction was stirred at 0° C. for 1 h before diluting with water (5 mL) and acidifying to pH3 with KHSO₄ (aq.). The aqueous layer was extracted with DCM (4×15 mL) and the combined organic extracts passed through a phase separator cartridge (Biotage) before concentrating in vacuo to provide the title compound I.

Method F: As method A except MeOH was used as the reaction solvent and aqueous K₂CO₃ (10% w/v) was added to a stirring solution of chlorophosphine gold(I) compound VII and thiol III. The resultant precipitate that formed during the reaction was collected by filtration and was washed with a combination of MeOH, EtOH, water, Et₂O or hexane to provide the title compound.

Method G: As method A except after stirring at 0° C. for 1 h, water was added and the resultant precipitate collected by filtration. The solid was washed with a combination of MeOH, EtOH, water, Et₂O or hexane to provide the title compound.

Method H: As method A except after aqueous work up, the product was purified by trituration.

Method I: As method E except after aqueous work up, the product was purified by trituration.

Method J: As method F except EtOH was used as the reaction solvent.

Method K: Thiol III (0.1 mmol) was dissolved in THF (10 mL) and NaH (60% dispersion in mineral oil, 0.2 mmol) added. The reaction mixture was stirred at rt for 15 mins whereupon chlorophosphine gold(I) compound VII (0.1 mmol) was added. The reaction mixture was stirred at rt for 18 h before water (10 mL) was added followed by aqueous KHSO₄ (2M) until pH 6 was reached. The aqueous layer was extracted using EtOAc (3×30 mL) and the combined organic extracts concentrated in vacuo to provide the crude product. Trituration with 1:1 Et₂O/isohexane provided title compound I.

Method L: As method A except MeOH was used as the reaction solvent.

Method M: The appropriate thiol III (0.32 mmol) was dissolved in EtOH (2.0 mL) and aqueous NaOH (1M, 2 mL) added. The reaction was then cooled to 0° C. and chlorophosphine gold(I) complex VII (0.32 mmol) was added in one portion. The reaction was stirred at 0° C. for 1 h whereupon the reaction was poured into water and extracted with DCM (×2). The combined organic extracts were washed with brine and passed through a phase separator cartridge (Biotage) and concentrated in vacuo to afford the product I.

The solvent (or combination of solvents) used for trituration and isolation of target compounds I can be selected from the following: MeOH, EtOH, water, Et₂O, EtOAc, isohexane or DCM.

Some of the compounds were prepared using methods in which minor modifications to the general methods were made; specifically, these methods involved small changes to the stoichiometry of reagents (1-2 equivalents), duration of reaction (1-18 h) and volume of solvent (1-2 mL).

The following compounds were made using these methods:

TABLE 1

| | Compound | | Analytical Data |
|---|---|---|---|
| Structure | Number | Method | Physical appearance/Yield |
| 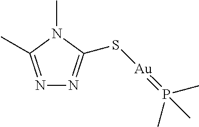 | 1 | A | ¹H-NMR (400 MHz, CDCl₃): δ ppm 3.48 (3H, s), 2.34 (3H, s), 1.61 (9H, d, J = 10.9 Hz) ³¹P-NMR (162 MHz, CDCl₃): δ ppm −3.64 (s) Cream solid; 122 mg, 94% |
| 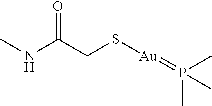 | 2 | A | ¹H-NMR (400 MHz, CDCl₃): δ ppm 7.50 (1H, br s), 3.60 (2H, s), 2.83 (3H, d, J = 4.8 Hz), 1.56 (9H, d, J = 10.6 Hz). ³¹P-NMR (162 MHz, CDCl₃): δ ppm −1.65 (s) White solid; 76 mg, 62% |
| 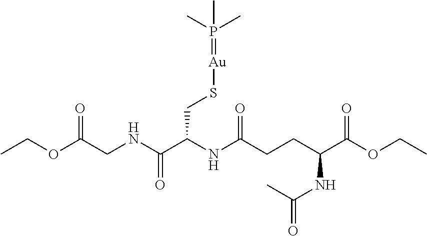 | 3 | B | ¹H-NMR (400 MHz, DMSO-d6): δ ppm 8.28 (1H, s), 8.26 (1H, s), 7.95 (1H, br s), 4.27 (1H, m), 4.20 (1H, m), 4.08 (2H, q, J = 7.1 Hz), 4.07 (2H, q, J = 7.1 Hz), 3.80 (2H, d, J = 6.1 Hz), 3.11 (1H, dd, J = 12.4, 4.0 Hz), 2.88 (1H, dd, J = 12.4, 9.3 Hz), 2.35-2.17 (2H, m), 1.95 (1H, m), 1.85 (3H, s), 1.79 (1H, m), 1.56 (9H, d, J = 11.1 Hz), 1.19 (3H, t, J = 7.1 Hz), 1.18 (3H, t, J = 7.1 Hz) ³¹P-NMR (162 MHz, DMSO-d6): δ ppm −0.09 (s) White solid; 87 mg, 58% |
| 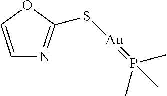 | 4 | B | ¹H-NMR (400 MHz, CDCl₃): δ ppm 7.47 (1H, m), 6.93 (1H, m), 1.62 (9H, d, J = 10.9 Hz). ³¹P-NMR (162 MHz, CDCl₃): δ ppm −2.37 (s) Yellow oil; 109 mg, 90% |
| 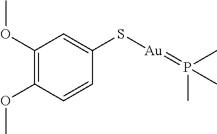 | 5 | A | ¹H-NMR (400 MHz, CDCl₃): δ ppm 7.08 (1H, dd, J = 8.4, 1.6 Hz), 7.06 (1H, d, J = 1.6 Hz), 6.64 (1H, d, J = 8.4 Hz), 3.82 (3H, s), 3.81 (3H, s), 1.56 (9H, d, J = 10.4 Hz). ³¹P-NMR (162 MHz, CDCl₃): δ ppm −1.76 (s) White solid; 101 mg, 94% |
| 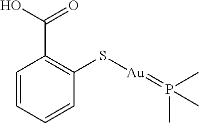 | 6 | E | ¹H-NMR (400 MHz, DMSO-d6): δ ppm 12.56 (1H, br s), 7.67 (1H, dd, J = 7.8, 0.8 Hz), 7.34 (1H, dd, J = 7.6, 1.3 Hz), 7.13 (1H, ddd, 1H, J = 7.8, 7.6, 1.5 Hz), 6.97 (1H, td, J = 7.6, 1.3 Hz) 1.60 (9H, d, J = 11.4 Hz). ³¹P-NMR (162 MHz, DMSO-d6): δ ppm 1.30 (s) Yellow solid; 105 mg, 76% |
| 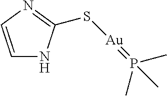 | 7 | A | ¹H-NMR (400 MHz, DMSO-d6): δ ppm 11.46 (1H, br s), 6.82 (1H, br s), 6.75 (1H, br s), 1.60 (9H, d, J = 11.4 Hz) ³¹P-NMR (162 MHz, DMSO-d6): δ ppm 0.01 (s) White solid; 42 mg, 35% |
| 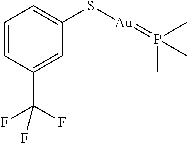 | 8 | B | ¹H-NMR (400 MHz, CDCl₃): δ ppm 7.82 (1H, m), 7.69 (1H, m), 7.26-7.16 (2H, m) 1.63 (9H, d, J = 10.6 Hz). ³¹P-NMR (162 MHz, CDCl₃): δ ppm −1.97 (s) White solid; 118 mg, 81% |
| 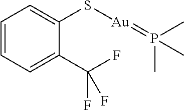 | 9 | B | ¹H-NMR (400 MHz, CDCl₃): δ ppm 7.87 (1H, d, J = 8.1 Hz), 7.54 (1H, dd, J = 7.8, 1.5 Hz), 7.17 (1H, m), 7.03 (1H, m), 1.59 (9H, d, J = 10.6 Hz). ³¹P-NMR (162 MHz, CDCl₃): δ ppm −1.57 (s) Colourless gum; 144 mg, 99% |
| 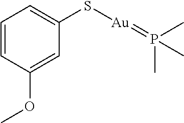 | 10 | B | ¹H-NMR (400 MHz, CDCl₃): δ ppm 7.16-7.11 (2H, m), 7.00 (1H, m), 6.56 (1H, ddd, J = 8.1, 2.4, 1.0 Hz), 3.75 (3H, s), 1.58 (9H, d, J = 10.6 Hz). ³¹P-NMR (162 MHz, CDCl₃): δ ppm −1.63 (s) White solid; 127 mg, 95% |
| 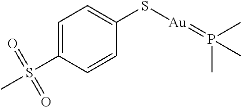 | 11 | A | ¹H-NMR (400 MHz, DMSO-d6): δ ppm 7.60-7.52 (4H, m), 3.11 (3H, s), 1.63 (9H, d, J = 11.4 Hz). ³¹P-NMR (162 MHz, DMSO- d6): δ ppm 1.84 (s) White solid; 61 mg, 82% |
| 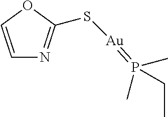 | 12 | B | ¹H-NMR (400 MHz, CDCl₃): δ ppm 7.41 (1H, d, J = 1.0 Hz), 6.87 (1H, d, J = 1.0 Hz), 1.82 (2H, dq, J = 10.4, 7.6 Hz), 1.52 (6H, d, J = 10.4 Hz), 1.17 (3H, dt, J = 20.0, 7.6 Hz). ³¹P-NMR (162 MHz, CDCl₃): δ ppm 10.46 (s) Brown oil; 42 mg, 94% |
| 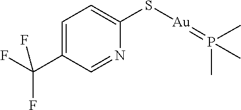 | 13 | B | ¹H-NMR (400 MHz, CDCl₃): δ ppm 8.50 (1H, br d, J = 2 .5 Hz), 7.53 (1H, d, J = 8.3 Hz), 7.48 (1H, dd, J = 8.3, 2.5 Hz), 1.65 (9H, d, J = 10.4 Hz). ³¹P-NMR (162 MHz, CDCl₃): δ ppm −1.44 (s) White solid; 75 mg, 40% |
| 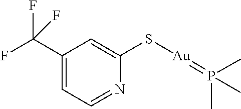 | 14 | B | ¹H-NMR (400 MHz, CDCl₃): δ ppm 8.40 (1H, d, J = 5.4 Hz), 7.68 (1H, s), 7.03 (1H, d, J = 5.4 Hz), 1.65 (9H, d, J = 10.6 Hz). ³¹P-NMR (162 MHz, CDCl₃): δ ppm −1.24 (s) Pale yellow solid; 85 mg, 45% |
| 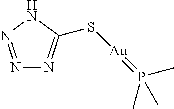 | 15 | E | ¹H-NMR (400 MHz, DMSO-d6): δ ppm 15.50 (1H, br s), 1.63 (9H, d, J = 11.6 Hz). ³¹P-NMR (162 MHz, DMSO-d6): δ ppm −1.00 (s) White solid; 56 mg, 93% |
| 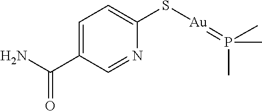 | 16 | A | ¹H-NMR (400 MHz, DMSO-d6): δ ppm 8.64 (1H, dd, J = 2.3, 0.8 Hz), 7.88 (1H, br s), 7.77 (1H, dd, J = 8.3, 2.3 Hz), 7.41 (1H, dd, J = 8.3, 0.8 Hz), 7.33 (1H, br s), 1.63 (9H, d, J = 11.4 Hz). ³¹P-NMR (162 MHz, DMSO-d6): δ ppm −0.08 (s) Brown solid; 14 mg, 50% |
| 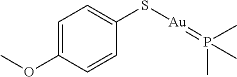 | 17 | A | ¹H NMR (400 MHz, CDCl₃): δ ppm 7.42 (2H, app d, J = 8.7 Hz), 6.68 (2H, app d, J = 8.7 Hz), 3.74 (3H, s), 1.57 (9H, d, J = 10.6 Hz). ³¹P-NMR (162 MHz, CDCl₃): δ ppm −2.35 (s). White solid; 38 mg, 57% |
| 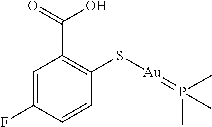 | 18 | E | ¹NMR (400 MHz, CDCl₃): δ ppm 13.50 (1H, br s), 8.02 (1H, dd, J = 9.9, 3.0 Hz), 7.59 (1H, dd J = 8.6, 5.6 Hz), 7.2 (1H, ddd, J = 8.6, 7.6, 3.0 Hz), 1.58 (9H, d, J = 10.9 Hz). ³¹P-NMR (162 MHz, CDCl₃): δ ppm −3.51 (s) Yellow solid; 66 mg, 92% |
| 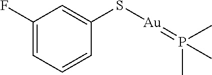 | 19 | A | ¹H-NMR (400 MHz, DMSO-d6): δ ppm 7.18 (1H, ddd, J = 7.8, 1.8, 1.0 Hz), 7.13- 7.03 (2H, m), 6.72 (1H, dddd, J = 10.9, 8.3, 2.8, 1.0 Hz), 1.61 (9H, d, J = 11.4 Hz). ³¹P-NMR (162 MHz, DMSO-d6): δ ppm 0.59 (s) White solid; 60 mg, 92% |
| 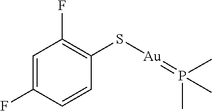 | 20 | A | ¹H-NMR (400 MHz, DMSO-d6): δ ppm 7.54 (1H, ddd, J = 9.3, 8.6, 6.6 Hz), 7.06 (1H, td, J = 9.3, 2.8 Hz), 6.84 (1H, tdd, J = 8.6, 2.8, 1.0 Hz), 1.60 (9H, d, J = 11.4 Hz). ³¹P-NMR (162 MHz, DMSO-d6): δ ppm 0.00 (s) White solid; 66 mg, 97% |
| 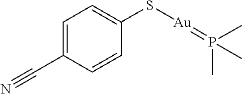 | 21 | C | ¹H-NMR (400 MHz, DMSO-d6): δ ppm 7.54-7.51 (2H, m), 7.46-7.43 (2H, m), 1.63 (9H, d, J = 11.4 Hz). ³¹P-NMR (162 MHz, DMSO-d6): δ ppm 0.69 (s) Off-white solid; 30 mg, 45% |
| 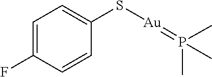 | 22 | A | ¹H-NMR (400 MHz, DMSO-d6): δ ppm 7.32 (2H, app dd, J = 8.8, 5.3 Hz), 6.92- 6.86 (2H, m), 1.60 (9H, d, J = 11.1 Hz). ³¹P-NMR (162 MHz, DMSO-d6): δ ppm 0.45 (s) White solid; 62 mg, 96% |
| 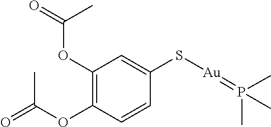 | 23 | D | ¹H-NMR (400 MHz, CDCl₃): δ ppm 7.35 (1H, dd, J = 8.6, 2.3 Hz), 7.30 (1H, d, J = 2.3 Hz), 6.88 (1H, d, J = 8.6 Hz), 2.24 (3H, s), 2.24 (3H, s), 1.58 (9H, d, J = 10.4 Hz). ³¹P-NMR (162 MHz, CDCl₃): δ ppm −2.46 (s) Pale pink solid; 15 mg, 60% |
| 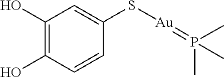 | 24 | D | ¹H-NMR (400 MHz, DMSO-d6): δ ppm 8.61 (1H, br s), 8.38 (1H, br s), 6.72 (1H, d, J = 2.3 Hz), 6.55 (1H, dd, J = 8.1, 2.3 Hz), 6.44 (1H, d, J = 8.1 Hz), 1.58 (9H, d, J = 11.1 Hz). ³¹P-NMR (162 MHz, DMSO-d6): δ ppm −0.09 (s) Pink oil; 44 mg, 64% |
| 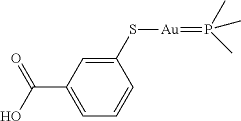 | 25 | E | ¹H-NMR (400 MHz, DMSO-d6): δ ppm 12.80 (1H, br s), 7.91 (1H, t, J = 1.8 Hz), 7.57 (1H, ddd, J = 7.8, 1.8, 1.0 Hz), 7.48 (1H, m), 7.16 (1H, t, J = 7.8 Hz), 1.61 (9H, d, J = 11.4 Hz). ³¹P-NMR (162 MHz, DMSO-d6): δ ppm 0.82 (s) White solid; 52 mg, 75% |
| 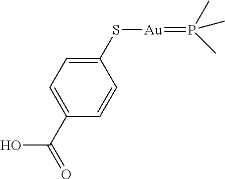 | 26 | E | ¹H-NMR (400 MHz, DMSO-d6): δ ppm 12.53 (1H, br s), 7.60 (2H, app d, J = 8.6 Hz), 7.44 (2H, app d, J = 8.6 Hz), 1.62 (9H, d, J = 11.4 Hz). ³¹P-NMR (162 MHz, DMSO-d6): δ ppm 0.89 (s) White solid; 39 mg, 56% |
| 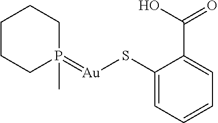 | 27 | E | ¹H-NMR (400 MHz, CDCl₃): δ ppm 13.35 (1H, br s), 8.29 (1H, dd, J = 7.8, 1.8 Hz), 7.58 (1H, dd, J = 7.8, 1.8 Hz), 7.22 (1H, ddd, J = 7.6, 7.3, 1.8 Hz), 7.13 (1H, ddd, J = 7.8, 7.3, 1.3 Hz), 2.05-1.95 (2H, m), 1.86-1.57 (7H, m), 1.50 (3H, d, J = 10.6 Hz) 1.31 (1H, m). ³¹P-NMR (162 MHz, CDCl₃): δ ppm 4.47 (s) White solid; 38 mg, 57% |
| 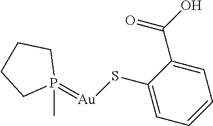 | 28 | E | ¹H-NMR (400 MHz, CDCl₃): δ ppm 13.33 (1H, br s), 8.27 (1H, dd, J = 7.8, 1.4 Hz), 7.58 (1H, dd, J = 7.8, 1.4 Hz), 7.22 (1H, ddd, J = 7.6, 7.3,1.8 Hz), 7.13 (1H, ddd, J = 7.8, 7.3, 1.4 Hz), 2.16-2.04 (2H, m), 1.96-1.74 (6H, m), 1.42 (3H, d, J = 10.6 Hz). ³¹P-NMR (162 MHz, CDCl₃): δ ppm 18.38 (s) White solid; 53 mg, 82% |
| 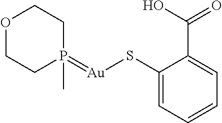 | 29 | E | ¹H-NMR (400 MHz, CDCl₃): δ ppm 13.24 (1H, br s), 8.35 (1H, dd, J = 7.8, 1.3 Hz), 7.67 (1H, dd, J = 7.8, 1.3 Hz), 7.32 (1H, ddd, J = 7.6, 7.3, 1.8 Hz), 7.22 (1H, ddd, J = 7.8, 7.3, 1.3 Hz), 4.15-4.01 (2H, m), 3.99-3.88 (2H, m), 2.21-2.14 (2H, m), 2.08-1.96 (2H, m), 1.72 (3H, d, J = 10.6 Hz). ³¹P-NMR (162 MHz, CDCl₃): δ ppm −1.49 (s) Beige solid; 39 mg, 59% |
| 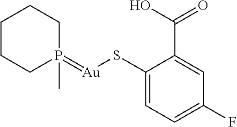 | 30 | E | ¹H-NMR (400 MHz, CDCl₃): δ ppm 13.26 (1H, br s), 8.02 (1H, dd, J = 10.1, 3.1 Hz), 7.59 (1H, dd, J = 8.6, 5.6 Hz), 7.02 (1H, ddd, J = 8.6, 7.6, 3.1 Hz), 2.12-2.01 (2H, m), 1.96-1.72 (6H, m), 1.66 (1H, m), 1.58 (3H, d, J = 10.6 Hz), 1.44-1.34 (1H, m). ³¹P-NMR (162 MHz, CDCl₃): δ ppm 4.57 (s) White solid; 45 mg, 65% |
| 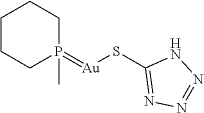 | 31 | A | ¹H-NMR (400 MHz, CDCl₃): δ ppm 15.46 (1H, br s), 2.18-2.04 (2H, m), 2.02- 1.72 (6H, m), 1.56 (1H, m), 1.67 (3H, d, J = 11.4 Hz), 1.38 (1H, m). ³¹P-NMR (162 MHz, CDCl₃): δ ppm 6.86 (s) Beige solid; 31 mg, 76% |
| 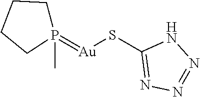 | 32 | A | ¹H-NMR (400 MHz, CDCl₃): δ ppm 15.53 (1H, br s), 2.20-2.12 (2H, m), 2.06- 1.94 (2H, m), 1.93-1.83 (4H, m), 1.58 (3H, d, J = 11.1 Hz). ³¹P-NMR (162 MHz, CDCl₃): δ ppm 20.32 (s) White solid; 30 mg, 66% |
| 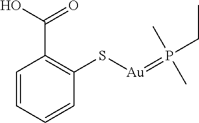 | 36 | E | ¹H-NMR (400 MHz, CDCl₃): δ ppm 13.70 (1H, br s), 8.32 (1H, dd, J = 7.8, 1.8 Hz), 7.63 (1H, dd, J = 7.8, 1.3 Hz), 7.29 (1H, ddd, J = 7.8, 7.3, 1.8 Hz), 7.20 (1H, ddd, J = 7.8, 7.3, 1.3 Hz), 1.84 (2H, dq, J = 10.4, 7.7 Hz,), 1.70 (6H, d, J = 10.6 Hz), 1.19 (3H, dt, J = 20.5, 7.7 Hz). ³¹P-NMR (162 MHz, CDCl₃): δ ppm 9.71 (s) Brown gum; 60 mg, 87% |
| 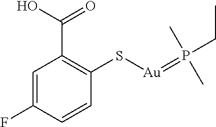 | 37 | E | ¹H-NMR (400 MHz, CDCl₃): δ ppm 13.7 (1H, br s), 8.02 (1H, dd, J = 9.9, 3.0 Hz), 7.58 (1H, dd, J = 8.6, 5.6 Hz), 7.02 (1H, ddd, J = 8.6, 7.6, 3.0 Hz), 1.84 (2H, dq, J = 10.6, 7.6 Hz), 1.54 (6H, d, J = 10.6 Hz), 1.19 (3H, dt, J = 20.5, 7.6 Hz). ³¹P-NMR (162 MHz, CDCl₃): δ ppm 9.62 (s) Brown gum; 81 mg, 58% |
| 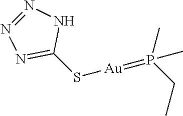 | 38 | E | ¹H-NMR (400 MHz, DMSO-d6): δ ppm 15.50 (1H, br s), 1.95 (2H, dq, J = 11.1,7.8 Hz), 1.61 (6H, d, J = 11.1 Hz), 1.13 (3H, dt, J = 20.2, 7.8 Hz). ³¹P-NMR (162 MHz, DMSO-d6): δ ppm 12.06 (s) White solid; 46 mg, 87% |
| 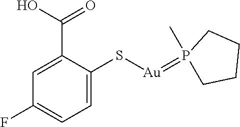 | 39 | E | ¹H-NMR (400 MHz, CDCl₃): δ ppm 13.66 (1H, br s), 8.04 (1H, dd, J = 9.8, 3.0 Hz), 7.58 (1H, dd, J = 8.6, 5.6 Hz), 7.22 (1H, ddd, J = 8.6, 7.6, 3.0 Hz), 2.23-2.11 (2H, m), 2.04-1.80 (6H, m), 1.48 (3H, d, J = 10.6 Hz). ³¹P-NMR (162 MHz, CDCl₃): δ ppm 18.32 (s) White solid; 54 mg, 78% |
| 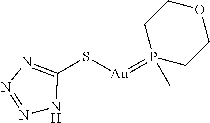 | 62 | K | ¹H-NMR (400 MHz, DMSO-d6): δ ppm 15.92 (1H, br s), 4.04-3.91 (2H, br m), 3.90-3.78 (2H, br m), 2.26-2.16 (2H, br m), 2.14-2.04 (2H, br m), 1.78 (3H, d, J = 11.4 Hz). ³¹P-NMR (162 MHz, DMSO-d6): δ ppm 0.13 (s) White solid; 18.3 mg, 44% |
| 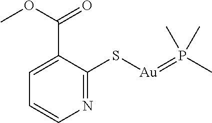 | 40 | A | ¹H-NMR (400 MHz, DMSO-d6): δ ppm 8.27 (1H, dd, J = 4.8, 1.9 Hz), 7.68 (1H, dd, J = 7.6, 1.9 Hz), 6.96 (1H, dd, J = 7.6, 4.8 Hz), 3.77 (3H, s), 1.60 (9H, d, J = 11.1 Hz). ³¹P- NMR (162 MHz, DMSO-d6): δ ppm −1.13 (s) Off-white gum; 54 mg, 54% |
| 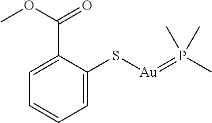 | 41 | L | ¹H-NMR (400 MHz, DMSO-d6): δ ppm 7.67 (1H, dd, J = 7.8, 1.0 Hz), 7.31 (1H, dd, J = 7.6, 1.5 Hz), 7.16 (1H, td, J = 7.6, 1.5 Hz), 6.98 (1H, td, J = 7.6, 1.0 Hz), 3.76 (3H, s), 1.60 (9H, d, J = 11.4 Hz). ³¹P-NMR (162 MHz, DMSO-d6): δ ppm 0.14 (s) White solid; 88 mg, 96% |
| 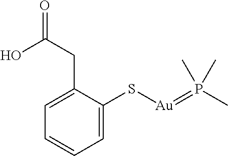 | 42 | E | ¹H-NMR (400 MHz, DMSO-d6): δ ppm 12.05 (1H, br s), 7.59 (1H, dd, J = 7.6, 1.3 Hz), 7.09 (1H, dd, J = 7.6, 1.5 Hz), 7.97 (1H, td, J = 7.6, 1.5 Hz), 6.90 (1H, td, J = 7.6, 1.3 Hz), 3.74 (2H, s), 1.59 (9H, d, J = 11.4 Hz). ³¹P-NMR (162 MHz, DMSO-d6): δ ppm −0.15 (s) White solid; 53 mg, 75% |
| 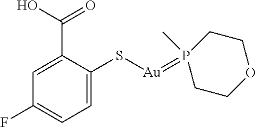 | 43 | E | ¹H-NMR (400 MHz, CDCl₃): δ ppm 8.02 (1H, br d), 7.52 (1H, ddd, J = 8.6, 5.6, 1.8 Hz), 6.97 (1H, m), 4.05-4.01 (2H, m), 3.91- 3.80 (2H, m), 2.15-2.04 (2H, m), 2.00-1.87 (2H, m), 1.62 (3H, d, J = 10.6 Hz). ³¹P- NMR (162 MHz, CDCl₃): δ ppm 1.95 (s) Light yellow solid; 21 mg, 43% |
| 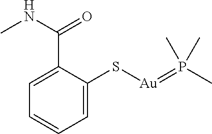 | 44 | L | ¹H-NMR (400 MHz, DMSO-d6): δ ppm 8.07 (1H, q, J = 4.8 Hz), 7.56 (1H, dd, J = 7.8, 1.0 Hz), 7.20 (1H, dd, J = 7.6, 1.5 Hz), 7.08 (1H, td, J = 7.6, 1.5 Hz), 6.96 (1H, td, J = 7.6, 1.0 Hz), 2.73 (3H, d, J = 4.8 Hz), 1.59 (9H, d, J = 11.1 Hz). ³¹P-NMR (162 MHz, DMSO-d6): δ ppm −0.28 (s) White solid; 66 mg, 93% |
| 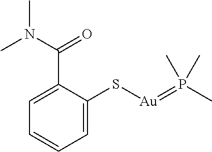 | 45 | L | ¹H-NMR (400 MHz, DMSO-d6): δ ppm 7.52 (1H, d, J = 7.6 Hz), 7.07 (1H, ddd, J = 7.6, 6.1, 2.8 Hz), 7.00-6.94 (2H, m), 2.96 (3H, s), 2.78 (3H, s), 1.59 (9H, d, J = 11.1 Hz). ³¹P-NMR (162 MHz, DMSO-d6): δ ppm −0.10 (s) White solid; 86 mg, 76% |
| 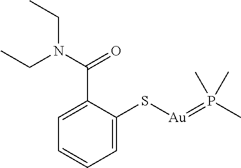 | 46 | L | ¹H-NMR (400 MHz, DMSO-d6): δ ppm 7.59 (1H, d, J = 7.8 Hz), 7.12 (1H, ddd, J = 7.8, 6.1, 3.0 Hz), 7.05-7.00 (2H, m), 3.70 (1H, dq, J = 14.1, 7.1 Hz), 3.27 (1H, dq, J = 14.1, 7.1 Hz), 3.15 (2H, qd, J = 7.1, 2.5 Hz), 1.65 (9H, d, J = 11.1 Hz), 1.22 (3H, t, J = 7.1 Hz), 1.02 (3H, t, J = 7.1 Hz). ³¹P-NMR (162 MHz, DMSO-d6): δ ppm −0.06 (s) Light yellow solid; 59 mg, 92% |
| 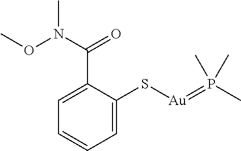 | 47 | L | ¹H-NMR (400 MHz, DMSO-d6): δ ppm 7.54 (1H, d, J = 7.8 Hz), 7.10 (1H, br s), 7.03 (1H, br d, J = 7.1 Hz), 6.97 (1H, br t, J = 7.1 Hz), 3.85-3.40 (3H, 2x br s), 3.30-3.00 (3H, 2x br s), 1.59 (9H, d, J = 11.4 Hz). ³¹P- NMR (162 MHz, DMSO-d6): δ ppm −0.02 (s) White solid; 103 mg, 94% |
| 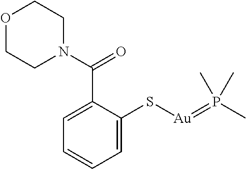 | 48 | L | ¹H-NMR (400 MHz, DMSO-d6): δ ppm 7.53 (1H, d, J = 7.8 Hz), 7.09 (1H, ddd, J = 7.8, 5.8, 3.0 Hz), 7.02-6.94 (2H, m), 3.77 (1H, ddd, J = 11.1, 6.6, 3.3 Hz), 3.72-3.63 (2H, m), 3.62-3.58 (2H, m), 3.46 (1H, ddd, J = 11.1, 6.6, 3.3 Hz), 3.22 (1H, ddd, J = 13.4, 6.6, 3.3 Hz), 3.05 (1H, ddd, J = 13.4, 6.6, 3.3 Hz), 1.59 (9H, d, J = 11.1 Hz). ³¹P-NMR (162 MHz, DMSO-d6): δ ppm −0.13 (s) White solid; 82 mg, 94% |
| 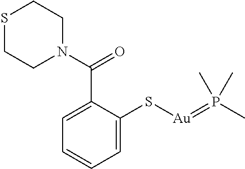 | 49 | L | ¹H-NMR (400 MHz, DMSO-d6): δ ppm 7.53 (1H, d, J = 7.8 Hz), 7.09 (1H, ddd, J = 7.8, 5.6, 3.5 Hz), 7.02-6.95 (2H, m), 3.95-3.80 (2H, m), 3.42 (1H, ddd, J = 13.6, 7.3, 2.8 Hz), 3.32 (1H, ddd, J = 13.6, 7.3, 2.8 Hz), 2.93 (1H, ddd, J = 13.1, 7.3, 2.8 Hz), 2.78- 2.62 (2H, m), 2.43 (1H, ddd, J = 13.1, 7.3, 2.8 Hz), 1.60 (9H, d, J = 11.4 Hz). ³¹P-NMR (162 MHz, DMSO-d6): δ ppm −0.14 (s) Off-white solid; 94 mg, 96% |
| 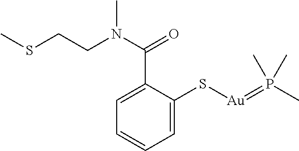 | 50 | F | ¹H-NMR (400 MHz, DMSO-d6): δ ppm 7.52 (1H, d, J = 7.8 Hz), 7.08 (1H, d, J = 7.6, 1.8 Hz), 7.04-6.94 (2H, m), 3.60 (1H, t, J = 7.8 Hz), 3.33-3.27 (0.5H, m), 3.23-3.15 (0.5H, m), 2.96 (1.5H, s), 2.81 (1.5H, s), 2.80-2.66 (1.5H, m), 2.47-2.40 (0.5H, m), 2.14 (1.5H, s), 1.70 (1.5H, s), 1.59 (9H, d, J = 11.1 Hz). ³¹P-NMR (162 MHz, DMSO-d6): δ ppm −0.12 (s) White solid; 28 mg, 46% |
| 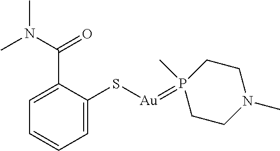 | 51 | G | ¹H-NMR (400 MHz, DMSO-d6): δ ppm 7.52 (1H, d, J = 7.8 Hz), 7.07 (1H, ddd, J = 7.8, 6.2, 2.8 Hz), 7.00-6.95 (2H, m), 2.96 (3H, s), 2.78 (3H, s), 2.74-2.55 (4H, m), 2.21 (3H, s), 2.16-2.00 (4H, m), 1.65 (3H, d, J = 11.1 Hz). ³¹P-NMR (162 MHz, DMSO-d6): δ ppm 0.74 (s) White solid; 51 mg, 40% |
| 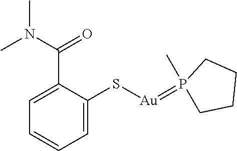 | 54 | G | ¹H-NMR (400 MHz, DMSO-d6): δ ppm 7.50 (1H, d, J = 7.8 Hz), 7.07 (1H, ddd, J = 7.8, 6.3, 2.5 Hz), 7.00-6.95 (2H, m), 2.95 (3H, s), 2.78 (3H, s), 2.14-2.07 (2H, m), 2.00- 1.92 (2H, m), 1.90-1.82 (4H, m), 1.52 (3H, d, J = 10.9 Hz). ³¹P-NMR (162 MHz, DMSO-d6): δ ppm 20.89 (s) White solid; 36 mg, 47% |
| 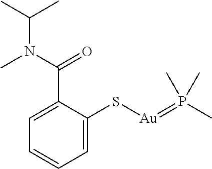 | 55 | F | ¹H-NMR (400 MHz, DMSO-d6): δ ppm 7.53 (1H, t, J = 7.8 Hz), 7.06 (1H, m), 7.00- 6.91 (2H, m), 4.81 (0.5H, quint., J = 6.8 Hz), 3.59 (0.5H, quint., J = 6.8 Hz), 2.80 (1.5H, s), 2.61 (1.5H, s), 1.59 (9H, d, J = 11.4 Hz), 1.24 (1.5H, d, J = 6.8 Hz), 1.12 (1.5H, d, J = 6.8 Hz), 0.95 (1.5H, d, J = 6.8 Hz). ³¹P-NMR (162 MHz, DMSO-d6): δ ppm −0.07 (s) White solid; 70 mg, 73% |
| 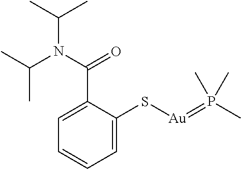 | 56 | F | ¹H-NMR (400 MHz, DMSO-d6): δ ppm 7.53 (1H, br d, J = 7.3 Hz), 7.03 (1H, td, J = 7.3, 1.5 Hz), 6.94 (1H, td, J = 7.3, 1.0 Hz), 6.88 (1H, dd, J = 7.3, 1.5 Hz), 3.56- 3.45 (2H, m), 1.59 (9H, d, J = 11.1 Hz), 1.48 (3H, d, J = 6.8 Hz), 1.44 (3H, d, J = 6.8 Hz), 1.25 (3H, d, J = 6.8 Hz), 0.95 (3H, d, J = 6.8 Hz). ³¹P-NMR (162 MHz, DMSO-d6): δ ppm −0.11 (s) White solid; 68 mg, 75% |
| 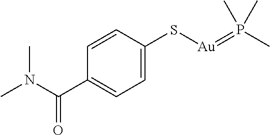 | 57 | F | ¹H-NMR (400 MHz, DMSO-d6): δ ppm 7.38 (2H, app d, J = 8.1 Hz), 7.11 (2H, app d, J = 8.1 Hz), 2.93 (6H, s), 1.62 (9H, d, J = 11.4 Hz). ³¹P-NMR (162 MHz, DMSO-d6): δ ppm 0.67 (s) Off-white solid; 57 mg, 46% |
| 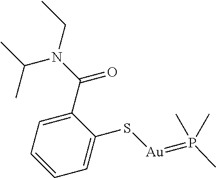 | 59 | L | ¹H-NMR (400 MHz, DMSO-d6): 7.53 (1H, m), 7.05 (1H, td, J = 7.8, 2.0 Hz), 6.99-6.90 (2H, m), 4.61 (0.3H, quint, J = 6.8 Hz), 3.56 (0.7H, quint, J = 6.8 Hz), 3.44-3.38 (0.7H, m), 3.26-3.17 (0.7H, m), 3.12-3.05 (0.6H, m), 1.59 (9H, d, J = 11.4 Hz), 1.28-1.16 (6H, m), 0.95 (2H, d, J = 6.6 Hz), 0.88 (1H, t, J = 7.1 Hz). ³¹P-NMR (162 MHz, DMSO- d6): δ ppm −0.05 (s) Pale yellow solid, 15 mg, 18% |
| 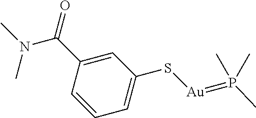 | 60 | L | ¹H-NMR (400 MHz, DMSO-d6): 7.41 (1H, dt, J = 7.8, 1.5 Hz), 7.30 (1H, t, J = 1.5 Hz), 7.11 (1H, t, J = 7.8 Hz), 6.92 (1H, dt, J = 7.8, 1.5 Hz), 2.95 (3H, br s), 2.89 (3H, br s), 1.61 (9H, d, J = 11.1 Hz). ³¹P-NMR (162 MHz, DMSO-d6): δ ppm 0.62 (s) White solid, 32 mg, 30% |
| 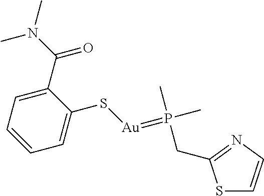 | 61 | H | ¹H-NMR (400 MHz, CDCl₃): 7.72 (1H, d, J = 3.5 Hz), 7.55 (1H, d, J = 7.3 Hz), 7.30-7.20 (2H, m), 7.10-7.00 (2H, m), 3.65 (2H, d, J = 10.4 Hz), 3.12 (3H, s), 2.93 (3H, s), 1.63 (6H, d, J = 10.1 Hz). ³¹P-NMR (162 MHz, CDCl₃): δ ppm 11.59 (s) White solid, 12 mg, 69% |
| 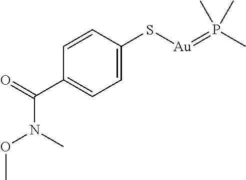 | 63 | L | ¹H-NMR (400 MHz, DMSO-d6): 7.40 (2H, app d, J = 8.6 Hz), 7.34 (2H, app d, J = 8.6 Hz), 3.54 (3H, s), 3.22 (3H, s), 1.62 (9H, d, J = 11.4 Hz). ³¹P-NMR (162 MHz, DMSO- d6): δ ppm 0.72 (s) Yellow solid, 107 mg, 80% |
| 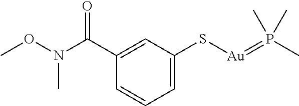 | 64 | L | ¹H-NMR (400 MHz, DMSO-d6): 7.52 (1H, br s), 7.45 (1H, dt, J = 6.6, 2.0 Hz), 7.15- 7.08 (2H, m), 3.56 (3H, s), 3.22 (3H, s), 1.61 (9H, d, J = 11.4 Hz). ³¹P-NMR (162 MHz, DMSO-d6): δ ppm 0.59 (s) Pale yellow gum, 94 mg, 75% |
| 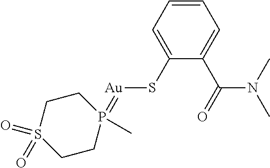 | 69 | A | ¹H-NMR (400 MHz, DMSO-d6): 7.49 (1H, app.d, 8.0 Hz), 7.15 (1H, ddd, 8.0, 6.4, 2.8 Hz), 7.00-6.90 (2H, m), 3.55-3.45 (4H, m), 2.98 (3H, s), 2.79 (3H, s), 2.60-2.50 (4H, m), 1.77 (3H, d, J = 11.1 Hz). ³¹P-NMR (162 MHz, DMSO-d6): δ ppm 2.82 (s) Beige solid, 24 mg, 98% |
| 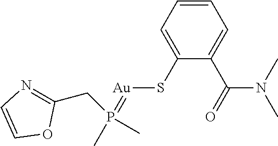 | 70 | I | ¹H-NMR (400 MHz, CDCl₃): 7.63 (1H, s), 7.56 (1H, app.d, 8.1 Hz), 7.15-7.00 (4H, m), 3.44 (2H, d, 9.9 Hz), 3.12 (3H, s), 2.93 (3H, s), 1.61 (6H, d, J = 10.1 Hz). ³¹P-NMR (162 MHz, CDCl₃): δ ppm 7.92 (s) White solid, 45 mg, 76% |
| 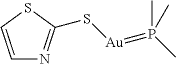 | 58 | L | ¹H-NMR (400 MHz, DMSO-d6): 7.38 (1H, d, J = 3.5 Hz), 7.11 (1H, d, J = 3.5 Hz), 1.63 (9H, d, J = 11.4 Hz). ). ³¹P-NMR (162 MHz, DMSO-d6): δ ppm −0.62 (s) Yellow oil, 39 mg, 61% |
| 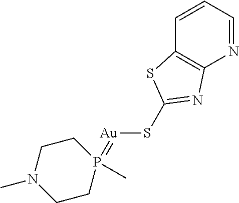 | 66 | J | ¹H-NMR (400 MHz, DMSO-d6): 8.41 (1H, app.d, J = 4.8 Hz), 8.20 (1H, app.d, J = 7.8 Hz), 7.20 (1H, dd, J = 7.8, 4.8 Hz), 2.90- 2.75 (2H, m), 2.70-2.60 (2H, m), 2.25 (3H, s), 2.25-2.10 (4H, m), 1.73 (3H, d, J = 10.9 Hz). ³¹P-NMR (162 MHz, DMSO-d6): δ ppm 1.44 (s) White solid, 16 mg, 43% |
| 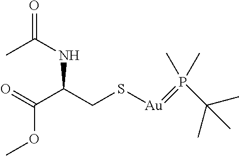 | 76 | L | ¹H NMR (400 MHz, CDCl₃): 6.72, (1H, br d, J = 7.3 Hz), 4.82 (1H, dt, J = 7.6, 4.8 Hz), 3.78 (3H, s), 3.48 (1H, dd, J = 13.1, 4.8), 3.36 (1H, dd, J = 13.1, 4.8 Hz), 2.10 (3H, s), 1.50 (6H, d, J = 9.6 Hz), 1.25 (9H, d, J = 16.2 Hz). ³¹P-NMR (162 MHz, CDCl₃): δ ppm 31.60 (s) Brown Solid, 96 mg, 89% |
| 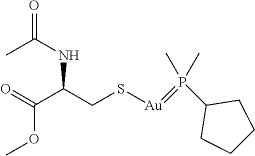 | 77 | A | ¹H NMR (400 MHz, CDCl₃): 6.65, (1H, br d, J = 7.3 Hz), 4.77 (1H, dt, J = 7.8, 4.5 Hz), 3.75 (3H, s), 3.45 (1H, dd, J = 13.1, 4.5), 3.30 (1H, dd, J = 13.1, 4.5 Hz), 2.09 (1H, m), 2.06 (3H, s), 2.10-1.88 (2H, m), 1.85- 1.60 (6H, m), 1.51 (6H, d, J = 9.9 Hz). ³¹P- NMR (162 MHz, CDCl₃): δ ppm 20.05 (s) Colourless gum, 33 mg, 94% |
| 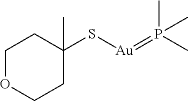 | 80 | M | ¹H NMR (400 MHz, CDCl₃): 4.00-4.08 (2H, m), 3.68 (2H, td, J = 4.8, 11.6 Hz), 1.86- 1.72 (4H, m), 1.61 (3H, s), 1.59 (9H, d, J = 9.6 Hz). ³¹P-NMR (162 MHz, CDCl₃): δ ppm −1.97 (s) White solid, 55 mg, 43% |

Compounds of the formula I, synthesised from thiol precursors IV, V, VI and IX, were synthesised via a one-pot, two-step procedure comprising thiol deprotection and coupling in situ to chloro(trialkyl phosphine) gold(I) complex VII.

### EXAMPLE 2

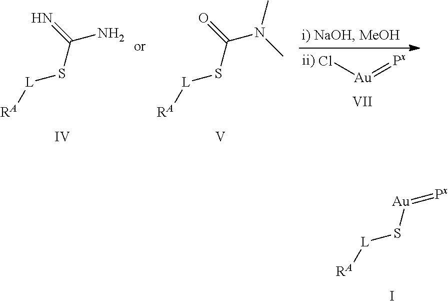

The appropriate protected thiol IV or V (0.33 mmol) was dissolved in MeOH (1 mL) and aqueous NaOH (10% w/v, 0.3 mL) added in one portion. The reaction was heated to 100° C. in a microwave reactor for 1 h, whereupon the reaction was cooled to 0° C. and the chlorophosphine gold(I) compound VII (0.33 mmol) added in one portion. The reaction was stirred at 0° C. for 1 h before it was diluted with water (5 mL) and extracted with DCM (4×15 mL). The combined organic extracts were passed through a phase separator cartridge (Biotage) and the solvent evaporated to provide the title compound I.

The following compounds were made using this method:

TABLE 2

| | Compound | Analytical Data |
|---|---|---|
| Structure | Number | Physical appearance/Yield |
| 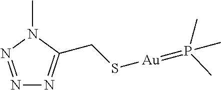 | 33 | ¹H-NMR (400 MHz, DMSO-d6): δ ppm 4.14 (2H, s), 4.08 (3H, s), 1.52 (9H, d, J = 11.1 Hz). ³¹P-NMR (162 MHz, DMSO-d6): δ ppm −0.04 (s) Off-white solid; 38 mg, 29% |
| 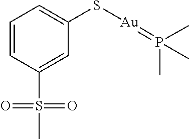 | 34 | ¹H-NMR (400 MHz, DMSO-d6): δ ppm 7.84 (1H, t, J = 1.8 Hz), 7.68 (1H, ddd, J = 7.8, 1.8, 1.0 Hz), 7.45 (1H, ddd, J = 7.8, 1.8, 1.0 Hz), 7.32 (1H, t, J = 7.8 Hz), 3.17 (3H, s), 1.62 (9H, d, J = 11.4 Hz). ³¹P-NMR (162 MHz, DMSO- d6): δ ppm 0.60 (s) Yellow solid; 30 mg, 65% |
| 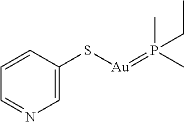 | 82 | ¹H-NMR (400 MHz, CDCl₃): 8.82 (1H, br d, J = 2 Hz), 8.17 (1H, br dd, J = 4.8, 1.5 Hz), 7.78 (1H, ddd, J = 8.1, 2.3, 1.5 Hz), 7.02 (1H, ddd, J = 8.1, 4.8, 0.8 Hz), 1.88 (2H, dq, J = 10.4, 7.6 Hz), 1.57 (6H, d, J = 10.4 Hz), 1.25 (3H, dt, J = 20.2, 7.6 Hz). ³¹P-NMR (162 MHz, CDCl₃): δ ppm 11.47 (s) Yellow oil, 61 mg, 96% |

### EXAMPLE 3

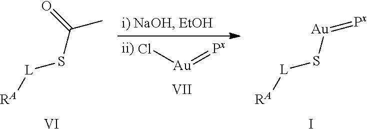

Under an atmosphere of nitrogen, the appropriate protected thiol VI (0.11 mmol) as a solution in degassed EtOH (1.0 mL) and aqueous NaOH (1 M, 1.0 mL) was added to chlorophosphine gold(I) compound VII (0.11 mmol) in one portion. The reaction was stirred at rt for 2 h whereupon water (10 mL) was added and the mixture extracted with DCM (3×10 mL). The combined organic extracts were passed through a phase separator cartridge (Biotage) and concentrated in vacuo to afford the crude product which was triturated with pentane/Et₂O (×2) to afford the title compound I.

Compound 78 was prepared and isolated as described in the general method except MeOH was used as the reaction solvent.

The following compounds were made using this method:

TABLE 3

| | Compound | Analytical Data |
|---|---|---|
| Structure | Number | Physical appearance/Yield |
| 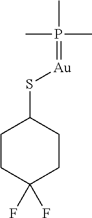 | 35 | ¹H-NMR (400 MHz, CDCl₃): δ ppm 3.31 (1H, m), 2.14-2.04 (4H, m), 1.79-1.64 (4H, m), 1.52 (9H, d, J = 10.6 Hz). ³¹P-NMR (162 MHz, CDCl₃): δ ppm −4.67 (s) Pink solid; 23 mg, 54% |
| 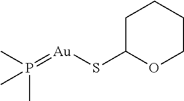 | 78 | ¹H NMR (400 MHz, CDCl₃): 5.27 (1H, dd, J = 3.0, 8.1 Hz), 4.14 (1H, m), 3.51 (1H, m), 2.08 (1H, m), 1.92 (1H, m), 1.73 (1H, m), 1.56-1.48 (3H, m), 1.54 (9H, d, J = 10.4 Hz). ³¹P-NMR (162 MHz, CDCl₃): δ ppm −0.39 (s) White solid, 74 mg, 84% |
| 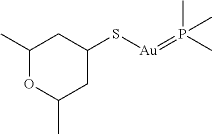 | 79* | ¹H NMR (400 MHz, CDCl₃): 4.35- 4.26 (2H, m), 3.99 (1H, qn, J = 3.6 Hz), 1.60-1.72 (4H, m), 1.55 (9H, d, J = 10.4 Hz), 1.14 (6H, d, J = 6.0 Hz), ³¹P-NMR (162 MHz, CDCl₃): δ ppm −0.34 (s) Yellow oil, 61 mg, 96% |
| 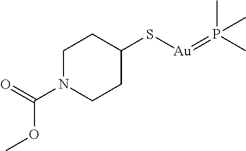 | 87 | ¹H NMR (400 MHz, CDCl₃): 4.07 (2H, br s), 3.67 (3H, s), 3.33 (1H, m), 2.88-2.82 (2H, m), 2.08-2.06 (2H, m), 1.61-1.47 (2H, m), 1.55 (9H, d, J = 10.3 Hz). ³¹P-NMR (162 MHz, CDCl₃): δ ppm 0.87 (s) Yellow oil, 77 mg, 94% |
| 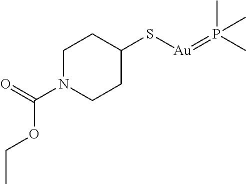 | 88 | ¹H NMR (400 MHz; CDCl₃): 4.14- 4.04 (2H m), 4.07 (2H, q, J = 7.1 Hz), 3.33 (1H, m), 2.87-2.80 (2H, m), 2.10-2.06 (2H, m), 1.61-1.55 (2H, m), 1.55 (9H, d, J = 10.4 Hz), 1.25 (3H, t, J = 7.1 Hz). ³¹P-NMR (162 MHz, CDCl₃): δ ppm −0.72 (s) White solid, 8 mg, 11% |
| 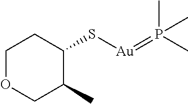 | 81** | ¹NMR (400 MHz, CDCl₃): 4.06 (1H, td, J = 5.2, 10.8 Hz), 3.78 (1H, dd, J = 5.2, 10.8 Hz), 3.72 (1H, m), 3.54-3.44 (2H, m), 1.96-1.84 (3H, m), 1.54 (9H, d, J = 10.4 Hz), 1.11 (3H, d, J = 7.2 Hz). ³¹P-NMR (162 MHz, CDCl₃): δ ppm 0.12 (s) Brown solid, 50 mg, 74% |

*mixture of diastereoisomers
**racemic

### EXAMPLE 4

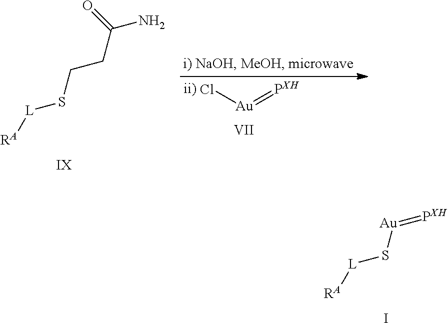

The appropriate protected thiol IX (0.18 mmol) was dissolved in MeOH (2.0 mL) and aqueous NaOH (10% w/v, 0.5 mL) added. The reaction mixture was heated to 100° C. in a microwave reactor for 1 h. The reaction was then cooled to 0° C. and chlorophosphine gold(I) complex VII (0.18 mmol) was added in one portion. The reaction was stirred at 0° C. for 1 h whereupon the reaction was poured into water (10 mL) and extracted with DCM (3×15 mL). The combined organic extracts were passed through a phase separator cartridge (Biotage) and concentrated in vacuo to afford the product I.

Compound 52 was prepared and isolated as described in the general method except after stirring at 0° C. for 1 h, water was added followed by acidification to pH 3 with aqueous KHSO₄ (2M).

The methyl ester in I-86 is also hydrolysed to the carboxylic acid during the reaction to prepare compound 52.

Compound 65 was prepared as described in the general method except the title compound was isolated by trituration.

The solvent (or combination of solvents) used for trituration and isolation of target compounds I can be selected from the following: MeOH, EtOH, water, Et₂O, EtOAc, isohexane or DCM.

The following compounds were made using this methods:

TABLE 4

| | Compound | Analytical Data |
|---|---|---|
| Structure | Number | Physical appearance/Yield |
| 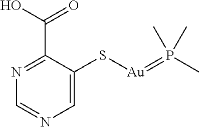 | 52 | ¹H-NMR (400 MHz, DMSO-d6): δ ppm 13.63 (1H, br s), 9.03 (1H, s), 8.71 (1H, s), 1.62 (9H, d, J = 11.6 Hz). ³¹P-NMR (162 MHz, DMSO-d6): δ ppm 0.35 (s) Off-white solid; 38 mg, 46% |
| 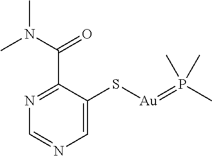 | 53 | ¹H-NMR (400 MHz, DMSO-d6): δ ppm 8.94 (1H, s), 8.73 (1H, s), 2.98 (3H, s), 2.76 (3H, s), 1.62 (9H, d, J = 11.4 Hz). ³¹P- NMR (162 MHz, DMSO-d6): δ ppm −0.40 (s) Beige solid; 30 mg, 38% |
| 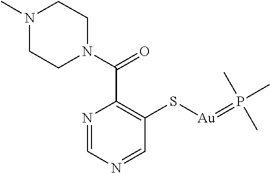 | 73 | ¹H-NMR (400 MHz, DMSO-d6): δ ppm 8.94 (1H, S), 8.71 (1H, S), 3.59 (2H, br t, J = 4.8 Hz), 3.06 (2H, br t, J = 4.8 Hz), 2.38 (2H, br t, J = 4.8 Hz), 2.33 (2H, br t, J = 4.8 Hz), 2.18 (3H, s), 1.61 (9H, d, J = 11.4 Hz). ³¹P- NMR (162 MHz, DMSO-d6): δ ppm 0.25 (s) Pink solid; 67 mg, 97% |
| 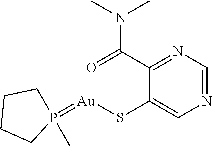 | 65 | ¹H-NMR (400 MHz, CDCl₃): δ ppm 8.94 (1H, s), 8.80 (1H, s), 3.14 (3H, s), 2.91 (3H, s), 2.30- 2.15 (2H, m), 2.03-1.80 (6H, m), 1.52 (3H, d, J = 10.6 Hz). ³¹P- NMR (162 MHz, CDCl₃): δ ppm 19.33 (s) Pink solid, 64 mg, 75% |
| 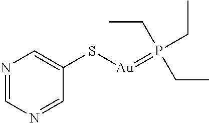 | 85 | ¹H-NMR (400 MHz, DMSO-d6): δ ppm 8.72 (2H, s), 8.70 (1H, s), 1.98 (6H, dq, J = 10.4, 7.6 Hz), 1.14 (9H, dt, J = 18.9, 7.6 Hz). ³¹P-NMR (162 MHz, DMSO-d6): δ ppm 39.68 (s) Grey solid, 70 mg. 96% |
| 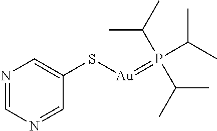 | 86 | ¹H-NMR (400 MHz, DMSO-d6): δ ppm 8.71 (2H, s), 8.70 (1H, s), 2.48 (3H, m), 1.27 (18H, dd, J = 15.9, 7.1 Hz). ³¹P-NMR (162 MHz, DMSO-d6): δ ppm 69.66 (s) Cream solid, 56 mg, 82% |
| 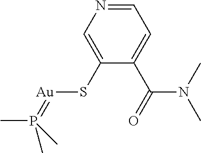 | 67 | ¹H-NMR (400 MHz, DMSO-d6): δ ppm 8.73 (1H, S), 8.13 (1H, d, J = 4.8 Hz), 6.99 (1H, d, J = 4.8 Hz), 2.96 (3H, s), 2.77 (3H, s), 160 (9H, d, J = 11.1 Hz). ³¹P- NMR (162 MHz, DMSO-d6): δ ppm 0.12 (s) Off-white solid, 81 mg, 98% |
| 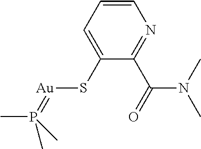 | 68 | ¹H-NMR (400 MHz, DMSO-d6): δ ppm 8.11 (1H, dt, J = 4.8, 1.7 Hz), 7.89 (1H, dt, J = 7.8, 1.7 Hz), 7.11 (1H, dd, J = 7.8, 4.8, 1.7 Hz), 2.96 (3H, d, J = 1.8 Hz), 2.72 (3H, d, J = 1.8 Hz), 1.60 (9H, d, J = 11.1, 1.4 Hz). ³¹P- NMR (162 MHz, DMSO-d6): δ ppm 0.06 (s) White solid, 15 mg, 35% |
| 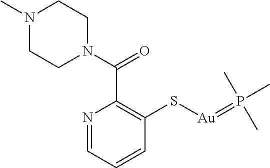 | 74 | ¹H-NMR (400 MHz, CDCl₃): δ ppm 8.22 (1H, app. d, J = 5.8 Hz), 7.86 (1H, d, J = 8.0 Hz), 7.01 (1H, dd, J = 8.0, 4.8 Hz), 3.90-3.85 (2H, m), 3.32 (2H, t, J = 5.0 Hz), 2.58 (2H, t, J = 5.0 Hz), 2.52-2.45 (2H, m), 2.34 (3H, s), 1.58 (9H, d, J = 10.9 Hz). ³¹P- NMR (162 MHz, CDCl₃): δ ppm −2.49 (s) White solid, 63 mg, 97% |
| 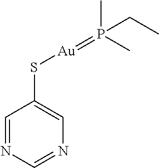 | 91 | ¹H-NMR (400 MHz, DMSO-d6): δ ppm 8.73 (2H, s), 8.70 (1H, s), 1.96 (2H, dq, J = 10.9, 7.6 Hz), 1.61 (6H, d, J = 10.9 Hz), 1.14 (3H, dt, J = 20.2, 7.6 Hz). ³¹P- NMR (162 MHz, DMSO-d6): δ ppm 13.64 (s) Cream solid, 68 mg, 93% |
| 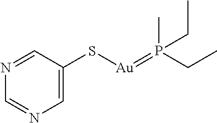 | 83 | ¹H-NMR (400 MHz, DMSO-d6): δ ppm 8.72 (2H, s), 8.70 (1H, s), 2.05-1.90 (4H, m), 1.59 (3H, d, J = 10.9 Hz), 1.14 (6H, dt, J = 19.5, 7.6 Hz). ³¹P-NMR (162 MHz, DMSO-d6): δ ppm 26.72 (s) Off-white solid, 82 mg, 91% |
| 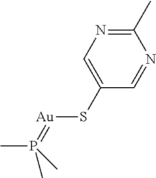 | 71 | ¹H-NMR (400 MHz, DMSO-d6): δ ppm 8.60 (2H, s), 2.48 (3H, s), 1.62 (9H, d, J = 11.4 Hz). ³¹P- NMR (162 MHz, DMSO-d6): δ ppm 0.73 (s) Grey solid, 54 mg, 86% |
| 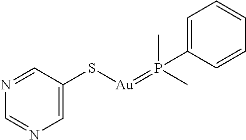 | 84 | ¹H-NMR (400 MHz, DMSO-d6): δ ppm 8.76 (2H, s), 8.71 (1H, s), 7-90-7.83 (2H, m), 7.60-7.56 (3H, m), 1.96 (6H, d, J = 10.9 Hz). ³¹P-NMR (162 MHz, DMSO-d6): δ ppm 12.72 (s) Cream solid, 78 mg, 92% |
| 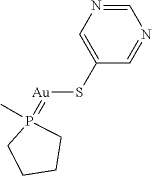 | 75 | ¹H-NMR (400 MHz, DMSO-d6): δ ppm 8.74 (2H, s), 8.70 (1H, s), 2.22-2.10 (2H, m), 2.04-1.93 (2H, m), 1.92-1.82 (4H, m), 1.58 (3H, d, J = 11.1 Hz). ³¹P-NMR (162 MHz, DMSO-d6): δ ppm 21.84 (s) Orange solid, 83 mg, 92% |
| 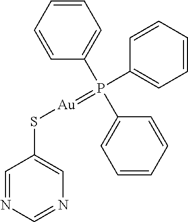 | 89 | ¹H-NMR (400 MHz, DMSO-d6): 8.80 (2H, s), 8.73 (1H, s), 7.69- 7.54 (15H, m). ³¹P-NMR (162 MHz, DMSO-d6): δ ppm 37.87 (s) Grey solid, 88 mg, 96% |
| 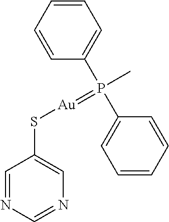 | 90 | ¹H-NMR (400 MHz, DMSO-d6): 8.79 (2H, s), 8.72 (1H, s), 7.82- 7.74 (4H, m), 7.60-7.54 (6H, m), 2.37 (3H, d, J = 10.6 Hz). ³¹P- NMR (162 MHz, DMSO-d6): δ ppm 25.13 (s) Colourless gum, 70 mg, 69% |

### EXAMPLE 4

Growth Media

Tryptic Soy Broth

| Formula/Litre | |
|---|---|
| Pancreatic Digest of Casein | 17.0 g |
| Enzymatic Digest of Soybean | 3.0 g |
| Sodium Chloride | 5.0 g |
| Di-potassium hydrogen Phosphate | 2.5 g |
| Glucose | 2.5 g |

Directions for use: Dissolve 30 g of the medium in one litre of purified water, mix thoroughly, and then autoclave at 121° C. for 15 minutes.

Luria Broth

| Formula/Litre | |
|---|---|
| Tryptone | 10.0 g |
| Yeast Extract | 5.0 g |
| NaCl | 5.0 g |

Directions for use: Dissolve components in 1 litre of distilled or deionized water and sterilize by autoclaving at 121° C. for 15 minutes.

Mueller Hinton II Broth (Cation-Adjusted)

| Formula/Litre | |
|---|---|
| Beef Extract | 3.0 g |
| Acid Hydrolysate of Casein | 17.5 g |
| Starch | 1.5 g |

*Adjusted and/or supplemented as required with appropriate salts to provide 20-25 mg/L of calcium and 10-12.5 mg/L of magnesium and as additionally required to meet performance criteria.

Directions for use: Dissolve components in 1 litre of distilled or deionized water and sterilize by autoclaving at 121° C. for 15 minutes.

Brain Heart Infusion Broth

| Formula/Litre | |
|---|---|
| Brain Heart Infusion solids | 12.5 g |
| Beef heart infusion solids | 5 g |
| Proteose peptone | 10 g |
| Glucose | 2 g |
| Sodium Chloride | 5 g |
| Di-sodium Phosphate | 2.5 g |

Directions for use: Dissolve components in 1 litre of purified water. Heat the mixture with frequent agitation to completely dissolve the medium, and sterilize by autoclaving at 121° C. for 15 minutes.

Growth assay for S.aureus.

Stock solution of the test compounds (20 mg/ml) in dimethyl sulfoxide (DMSO) were serially diluted in DMSO and each diluted compound added in duplicate to a 96-well plate to a final DMSO concentration of 2% (v/v). An overnight culture of S. aureus (Oxford strain) grown in tryptic soy broth (TSB) was diluted to approximately 5×10⁷ cfu/ml and 150 μl of this sample was added to each well of the 96-well plates. Control wells included an ‘untreated’ control with bacteria in TSB in the presence of 2% DMSO and a negative sample (containing 150p1 TSB growth media in the presence of 2% DMSO). Plates were incubated in a shaking incubator at 37° C. for 22-24 hours and bacterial growth assessed by absorbance at a wavelength of 595 nm. The minimum inhibitory concentration (MIC) was defined as the lowest concentration of compound that inhibited growth compared to the no-treatment control.

Variation of growth assays for:

Klebsiella pneumoniae, Acinetobacter baumannnii or E.coli (ATCC 25922): use of 1/100 overnight dilution to set up assay, medium used: Luria broth (LB); incubation without shaking.

P.aeruginosa (ATCC 27853): use of 1/100 overnight dilution to set up assay, medium used: Cation adjusted Mueller Hinton broth (CaMHB); incubation without shaking.

| | | K. | | P. |
|---|---|---|---|---|
| | S. aureus | pneumoniae | E. coli | aeruginosa |
| | MIC | MIC | MIC | MIC |
| Compound | (μg/mL) | (μg/mL) | (μg/mL) | (μg/mL) |
| 1 | 1.6 | 6.3 | 6.3 | 6.3 |
| 2 | 1.6-3.1 | 6.3 | 12.5 | 6.3 |
| 3 | 1.6 | 6.3 | 6.3 | 12.5 |
| 4 | 0.8 | 1.6 | 1.6 | 3.1 |
| 5 | 0.8-1.6 | 3.1 | 1.6 | 25 |
| 6 | 0.8 | 3.1-6.3 | 1.6-3.1 | 12.5-25 |
| 7 | 0.8 | 1.6-3.1 | 1.6 | 12.5 |
| 8 | 0.8 | 3.1 | 1.6 | 12.5 |
| 9 | 0.8-1.6 | 3.1 | 1.6 | 12.5 |
| 10 | 0.8-1.6 | 3.1 | 1.6-3.1 | 12.5 |
| 11 | 0.8 | 3.1-6.3 | 1.6-3.1 | 6.3-12.5 |
| 12 | 0.8 | 3.1-6.3 | 1.6-3.1 | 6.3-12.5 |
| 13 | 0.8-1.6 | 3.1 | 3.1-6.3 | 6.3-12.5 |
| 14 | 0.8-1.6 | 3.1-6.3 | 6.3-12.5 | 12.5 |
| 15 | 1.6 | 3.1 | 1.6-3.1 | 12.5 |
| 16 | 1.6 | 6.3 | 3.1 | 12.5 |
| 17 | 1.6 | 3.1 | 1.6-3.1 | 12.5 |
| 18 | 1.6 | 6.3 | 3.1 | 12.5 |
| 19 | 0.8 | 3.1-6.3 | 1.6-3.1 | 12.5 |
| 20 | 0.8 | 3.1-6.3 | 3.1-6.3 | 12.5 |
| 21 | 0.8 | 6.3 | 6.3 | 12.5 |
| 22 | 0.8 | 3.1-6.3 | 3.1 | 12.5 |
| 23 | 0.8-1.6 | 6.3 | 3.1-6.3 | 25 |
| 24 | 0.8 | 6.3 | 3.1-6.3 | 100 |
| 25 | <0.8 | 3.1-6.3 | 1.6 | |
| 26 | <0.8 | 3.1-6.3 | 1.6-3.1 | |
| 27 | 0.8-1.6 | 25 | 12.5 | |
| 28 | <0.8 | 6.3 | 12.5 | |
| 29 | 0.8-1.6 | 12.5 | 3.1 | |
| 30 | <0.8 | 12.5 | 3.1-6.3 | |
| 31 | 3.1 | 25-50 | 6.3 | |
| 32 | ≤0.8 | 3.1-6.3 | 1.6 | |
| 33 | 3.1 | 6.3 | 6.3 | 6.3 |
| 34 | 0.8 | 6.3 | 3.1-6.3 | 12.5-25 |
| 35 | 0.8-1.6 | 3.1-6.3 | 6.3 | 12.5 |
| 36 | <0/8 | 6.3 | 3.1 | |
| 37 | >0.8 | 6.3 | 3.1 | |
| 38 | <0.8 | | 3.1-6.3 | 1.6 |
| 39 | ≤0.8-1.6 | 3.1-6.3 | 1.6-6.3 | 25 |
| 40 | ≤0.8 | 1.6-3.1 | 0.8-1.6 | 12.5 |
| 41 | ≤0.8 | 1.6-3.1 | 0.8-1.6 | 6.3-12.5 |
| 42 | ≤0.8 | 3.1-6.3 | ≤0.8-1.6 | 12.5-25 |
| 43 | 1.6-3.1 | 12.5-25 | 6.3-12.5 | 50-100 |
| 44 | ≤0.8-3.1 | 50-100 | 1.6-12.5 | 25-50 |
| 45 | ≤0.8 | 3.1-6.3 | 1.6-3.1 | 6.3-12.5 |
| 46 | ≤0.8-1.6 | 3.1-6.3 | ≤0.8-1.6 | 12.5 |
| 47 | ≤0.8 | 6.3 | 1.6-25 | 6.3-25 |
| 48 | ≤0.8 | 6.3-12.5 | 1.6-3.1 | 12.5 |
| 49 | ≤0.8 | 6.3-12.5 | 1.6-3.1 | 12.5 |
| 50 | ≤0.8-1.6 | 6.3 | ≤0.8-3.1 | 6.3-12.5 |
| 51 | 1.6-6.3 | 6.3-25 | 6.3-12.5 | 6.1-12.5 |
| 52 | ≤0.8 | 6.1-12.5 | 0.8-3.1 | 6.3-100 |
| | | | | (*25 n = 8) |
| 53 | ≤0.8-3.1 | 3.1-12.5 | 1.6-3.1 | 6.3-25 |
| 54 | ≤0.8-3.1 | 6.1-12.5 | 3.1-12.5 | 12.5-25 |
| 55 | ≤0.8 | 6.3-12.5 | ≤0.8-1.6 | 6.3-12.5 |
| 56 | ≤0.8 | 12.5 | 1.6-3.1 | 6.3-12.5 |
| 57 | 0.8-1.6 | 6.3 | 1.6-3.1 | 6.3-12.5 |
| 58 | 1.6-3.1 | 3.1 | 1.6-3.1 | 3.1-6.3 |
| 59 | ≤0.8-3.1 | 6.3 | 1.6-3.1 | 6.3-25 |
| 60 | ≤0.8-1.6 | 6.3 | 1.6-3.2 | 6.3-12.5 |
| 61 | 1.6-3.1 | 25-50 | 12.5-25 | 25->100 |
| 62 | ≤0.8-3.1 | 12.5-25 | 3.1-12.5 | 50-100 |
| 63 | ≤0.8-1.6 | 3.1-6.3 | 1.6-6.3 | 6.3-12.5 |
| 64 | ≤0.8-1.6 | 3.1-6.3 | 1.6-3.2 | 6.3-12.5 |
| 65 | 0.8-1.6 | 12.5 | 3.1-6.3 | 6.3-25 |
| 66 | 3.1-6.3 | 3.1 | 3.1-6.3 | 6.3-12.5 |
| 67 | ≤0.8 | 12.5-25 | 1.6 | 3.1-12.5 |
| 68 | ≤0.8-12.5 | 6.3-25 | 1.6-12.5 | 12.5-25 |
| 69 | 3.1-6.3 | >100 | 12.5-25 | >100 |
| 70 | 1.6-3.1 | 25-50 | 6.3-12.5 | 25 |
| 71 | ≤0.8-1.6 | 1.6 | 1.6-3.1 | 6.3-25 |
| 73 | ≤0.8-1.6 | 6.3-12.5 | 3.1 | 25->100 |
| 74 | ≤0.8-1.6 | 12.5-25 | 3.1 | 6.3-12.5 |
| 75 | 1.6-3.1 | 1.6 | 6.3 | 6.3-25 |
| 76 | ≤0.8 | 50 | 25-50 | >100 |
| 77 | 1.6 | 25 | 25 | 50 |
| 78 | 6.3 | 100 | 100 | 100 |
| 79 | 6.3 | 12.5-25 | 12.5-25 | 12.5-25 |
| 80 | 0.8 | 2.3 | 3.1 | 6.3 |
| 81 | 0.8 | 3.1 | 3.1-6.3 | 6.3 |
| 82 | 0.8-1.6 | 3.1 | 2.4 | 6.3 |
| 83 | ≤0.8 | 3.1-6.3 | 3.1-6.3 | 3.1-12.5 |
| 84 | 1.6 | 3.1 | 6.3-12.5 | 12.5-50 |
| 85 | ≤0.8-1.6 | 25-50 | 12.5-25 | 12.5->100 |
| 86 | ≤0.8-1.6 | >100 | 50->100 | 50->100 |
| 87 | 1.2 | 6.3 | 3.1-6.3 | 12.5-25 |
| 88 | 0.8-1.6 | | 1.6-6.3 | |
| 90 | 1.6 | 25 | 12.5-50 | >100 |
| 91 | ≤0.4-1.6 | 3.1 | 1.6-6.3 | 1.6-6.3 |
| I-27 | 0.8 | 4.7 | 2.4 | 3.1 |
| I-30 | ≤0.8-6.3 | 3.1-6.3 | 1.6-6.3 | 6.3-12.5 |
| I-31 | 0.8 | 6.3 | 6.3-12.5 | 12.5-25 |
| I-34 | 1.2-3.1 | 6.3 | 3.1 | |
| I-36 | | | 6.3 | |
| I-39 | 1.6-3.1 | 6.3 | 6.3 | 12.5 |
| I-44 | 6.3-25 | 50-100 | 25-100 | 6.3-50 |
| I-49 | ≤0.8-1.6 | 12.5-25 | 6.3-12.5 | 25 |
| I-50 | 1.6-3.1 | 12.5-25 | 6.3-12.5 | 12.5-25 |
| I-52 | 0.8 | 6.3 | 6.3 | 6.3 |
| I-54 | | 12.5 | 12.5 | |
| I-130 | 0.8 | 9.4 | 6.3 | 9.4 |

*= geometic mean

Inhibition of Neisseria gonorrhoeae (NCTC 8375) growth on solid media

N. gonorrhoeae was grown for 48 hours at 37° C. on Chocolate agar plates (BD Diagnostics). A culture loop-full of bacterial culture was picked from the plate and re-suspended in 50 μl sterile phosphate buffered saline. The suspension was spread evenly onto the surface of a fresh chocolate agar plate and left to dry (approximately 5 minutes). Small discs of blotting paper were placed on the surface of the agar plate and 3 μl of test compounds (at 20 mg/ml) were applied to the discs. The plates were incubated overnight at 37 ° C. and zones of clearance around the disc were measured.

HepG2 Cell Inhibition Assay

Cell counting kit-8 (Sigma, CCK-8) assays were performed to assess the effect of compounds on cell viability. The assay is based on the reduction of a water-soluble tetrazolium salt (WST-8) by cellular dehydrogenases to a formazan dye which can be detected spectroscopically. 96-well plates were seeded with the human hepatocyte cell line (HepG2) at approximately 8×10³ cells per well in Eagle's Minimum Essential Medium (EMEM) with Earle's salts and sodium bicarbonate supplemented with 10% heat-inactivated foetal bovine serum 2 mM glutamine and 1% non-essential amino acids (NEAA). The following day serial dilutions of compounds (dissolved and diluted in DMSO) were added to the cells in duplicates. Control wells included an ‘untreated’ control where cells were grown in the presence of 1% DMSO and a ‘medium only’ control (plus 1% DMSO). After 24 hours CCK-8 reagent (10 μl) was added to each well and cell viability was assessed by measuring the absorbance at a wavelength of 450 nm after 2-3h hours. Only living cells can reduce the tetrazolium salts into coloured formazan products. Results were expressed as 50% growth inhibition (TD₅₀) values compared to ‘untreated’ control.

| | HepG2 cell |
|---|---|
| Compound | TD₅₀ (μg/mL) |
| 1 | 2 |
| 2 | 6 |
| 3 | 32 |
| 4 | 4 |
| 5 | 3 |
| 6 | 11.5 |
| 7 | 6 |
| 8 | 3 |
| 9 | 3 |
| 10 | 2 |
| 11 | 2 |
| 12 | 5 |
| 13 | 2.5 |
| 14 | 3 |
| 15 | 23 |
| 16 | 17 |
| 17 | 3.5 |
| 18 | 18 |
| 23 | 2 |
| 25 | 22 |
| 26 | 13 |
| 27 | 22 |
| 28 | 11 |
| 29 | 35 |
| 30 | 19 |
| 31 | 45 |
| 32 | 11 |
| 33 | 4 |
| 34 | 1 |
| 35 | 4 |

Efficacy Studies in the Galleria mellonella Model

G. mellonella larvae at 5^{th} or 6^{th} instar stage were purchased from a commercial supplier and used within 3 days. Prior to infection larvae were kept at room temperature. Larvae were infected with bacteria (various Gram positive and negative bacteria, including S.aureus, K.pneumoniae, E.coli and P.aeruginosa) using a sterile Hamilton syringe. Bacteria cultures were grown overnight, washed ×3 in PBS and resuspended in PBS. Larvae were wiped with 70% ethanol and 10 μl of bacteria solution (to cause 80%-100% death within 3-4 days) was injected into the bottom right proleg of the larvae. Larvae injected with 10 μl of PBS were used as negative controls. Larvae were then placed in petri dishes (1 dish per condition) containing filter paper at the bottom of the dish at 37° C. After various time points post infection (1-6h), larvae were taken from the incubator wiped again with 70% ethanol and injected with 10 μl of various concentrations of compound, dissolved in either 5% dimethyl sulfoxide, 5% ethanol or 5% 1-methyl-2-pyrrolidinone into a proleg on the left hand-side. Control larvae received 10 μl of 5% solvent. Ten larvae were injected for each condition. To assess the toxicity of the compound, larvae were injected with various concentrations of compound alone. Larvae were returned to a 37° C. incubator and checked daily. Larvae were considered dead when no movement occurred when touched with a blunt pair of forceps. Black or discoloured larvae which still showed movement were considered to be alive. Numbers of dead larvae were recorded each day.

### Primary Cells Viability Assay

Neutrophils and peripheral blood mononuclear cells (PBMCs) were isolated from venous blood obtained from healthy volunteers as previously described (Nauseef, Methods in Molecular Biology, 412 (2007), pp. 15-20). In brief, heparinised blood was diluted 1:1 with 3% Dextran-500 PBS solution (Sigma) to allow for erythrocyte sedimentation. Buffy coat was centrifuged over Hypaque-Ficoll (GE Lifescience) and PBMCs were carefully collected from the interface of the Hypaque-Ficoll and the upper liquid layer. Pelleted neutrophils were collected after hypotonic lysis of residing erythrocytes. Isolated cells were washed and suspended in culture media (RPMI+10% FBS) at 2× 106 cells/mL. Cell suspensions were transferred into 96-well plates containing compound serially diluted in DMSO (1% final volume). After 24 hours, the reaction was stopped and cells were stained with AnnexinV and 7-AAD. Results were determined by FACSCalibur and viability was defined for AnnexinV/7-AAD double negative cells population.

Biofilm Prevention Assay (S. aureus)

The effect of a test compound on the formation of a S. aureus biofilm was assessed using a biofilm prevention assay as described by Merritt et al. Current Protocols in Microbiology, 2011, 1B.1.1-1B.18 with slight modifications. Briefly, S. aureus was grown overnight in tryptic soy broth (TSB) and diluted to 1/100 before 150 μL was added to the wells of a flat bottomed 96-well plate. Three microliters of compound at the appropriate dilution in DMSO was added to the wells in duplicate. Controls included a positive control with bacteria alone in TSB with 2% DMSO and a negative (no bacteria) control with 150 μL TSB containing 2% DMSO. Plates were sealed with AeraSeal™ and incubated at 37° C. for 24 hours. Plates were then washed three times with PBS, dried at 60 ° C. for 1 hour and stained with crystal violet for 1 hour. The plates were again washed three times with water, then dried 33% acetic acid was added to re-solubilize the crystal violet stain bound to the adherent cells. Absorbance was then measured at 595 nm and expressed as a percentage of the bacteria only control. A biofilm inhibitory concentration (BIC₉₀) was determined as the concentration at which biofilm mass (measured by crystal violet staining) was reduced by at least 90% compared to untreated controls.

The effect of a test compound on preformed S. aureus biofilms can also be assessed. Briefly S.aureus was plated in 96-well plates as described above and incubated at 37° C. for 24 hours. Biofilms were then washed 3 times with TSB and 150 μL of fresh TSB and 3 μL of compound at the appropriate dilution in DMSO was added to the wells in duplicate. Plates were again sealed with AeraSeal™ and reincubated at 37° C. for 24 hours. Biofilm was then detected as described above.

| | S. aureus BF |
|---|---|
| | dispersal |
| Compound | BIC₉₀ (μg/mL) |
| 6 | ≤0.8 |
| 10 | ≤0.8 |
| 11 | ≤0.8 |
| 12 | 1.6 |
| 18 | ≤0.8 |
| 27 | ≤0.8 |
| 28 | ≤0.8 |
| 29 | ≤0.8 |
| 36 | ≤0.8 |

Biofilm Assay for A. baumannii

A.baumannii was grown overnight in LB broth and diluted 1/00- 1/500 before 200 μL was added to the wells of a flat bottomed 96-well plate with TSP 96 pins lid inserted. Plates with pins were incubated at 37° C. for 24 hours. Pins were washed with sterile phosphate buffered saline three times and exposed to compounds at pre-determined concentration in LB broth for 24 hours. Pins were washed again and either stained with crystal violet as described in the S.aureus biofilm assay, or incubated with LB media for 24 hours and the minimum biofilm eradication concentration (MBEC) was measured as the lowest concentration of compounds preventing further planktonic growth.

| Compound | Minimum biofilm eradication conc. (MBEC) μg/mL |
|---|---|
| 6 | 10 |
| 18 | 2.5 |
| 28 | 20 |
| 29 | 20 |
| 31 | 20 |
| 32 | 10 |
| 36 | 10 |
| 37 | 20 |
| 38 | 10 |
| 39 | 10 |
| 40 | 5 |
| 41 | 5 |
| 62 | 10 |

### Persister Cell Assay

To determine whether S. aureus persister cells were susceptible to treatment with a test compound, a persister cell (or SCV) isolate hemB mutant of NCTC 8325-4 was used (Von Eiff et al., (1997) J Bacteriol 179:4706-4712). This persister cell variant displays varying resistance to erythromycin and the aminoglycosides gentamicin and kanamycin. Growth assays were performed essentially as described above with the bacteria being grown in TSB. Disc assays were also performed by plating bacteria on TSB agar. Discs impregnated with an amount of test compound were placed on top of the agar. The plates were incubated overnight at 37° C. and any zone of bacterial inhibition was observed.

### Sensitivity of Multidrug Resistant Clinical Isolates

The activity of test compounds against multi-drug resistant bacterial strains was assessed by the disk diffusion assay; a standardised method to assess for the antimicrobial susceptibility of microorganisms (adapted from EUCAST, Version 5, January 2015). In brief, bacterial cultures were suspended in phosphate buffer and spread evenly onto blood agar plates. Cellulose disks were placed onto the agar plates and 3 μl test compound (60 μg/disk) were pipetted to the centre. A panel of standard antibiotics disks (Sigma) were used to control for the antimicrobial resistance profile of the individual strains (quantity as indicated in the table). The plates were then placed into a thermo-incubator and were cultured at 37° C. over-night. Activity was recorded by measuring the zone of clearance (mm) around the disks.

### REFERENCES

| | doi |
|---|---|
| Aguinagalde, L., et al., J. Antimicrob. Chemother., | 10.1093/jac/dkv163 |
| 2015, 70(9), 2608-2617 | |
| Harbut, M B, et al., PNAS, 2015, 112(14), 4453-4458 | 10.1073/pnas.1504022112 |
| Gli\check{s}ić, B D & Djuran MI, Dalton Trans., 2014, 43, 5950-5969 | 10.1039/c4dt00022f |
| Medeira, J M, et al., Inflammopharmacology, 2012, 20(6), | 10.1007/s10787-012- |
| 297-306 | 0149-1 |
| Jackson-Rosario, S, et al., J. Biol. Inorg. Chem., 2009, | 10.1007/s00775-009- |
| 14(4), 507-519 | 0466-z |
| Novelli, F, etal., Farmaco, 1999, 54, 232-236 | 10.1016/S0014- |
| | 827X(99)00019-1 |
| Shaw, C F, Chem Rev., 1999, 99(9), 2589-2600 | 10.1021/cr980431o |
| Rhodes, M D, etal., J. Inorg. Biochem., 1992, 46, 129-142 | 10.1016/0162- |
| | 0134(92)80016-O |
| Fricker, S P, Transition Met. Chem., 1996, 21, 377-383 | 10.1007/BF00139037 |
| Crooke et al., Biochemical Pharmacology, 1986, Vol. 35, | 10.1016/0006- |
| No. 20, 3423-3431 | 2952(86)90608-8 |
| Snyder et al., Biochemical Pharmacology, 1986, Vol. 35, | 10.1016/0006- |
| No. 6, 923-932 | 2952(86)90078-X |

1. A method of preventing or treating a bacterial infection by administering a compound of Formula (II) or a pharmaceutically acceptable salt, solvate or hydrate thereof: 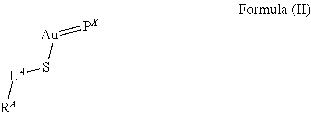
wherein P^{X} is selected from the group consisting of (P1), (P2) and (P3); 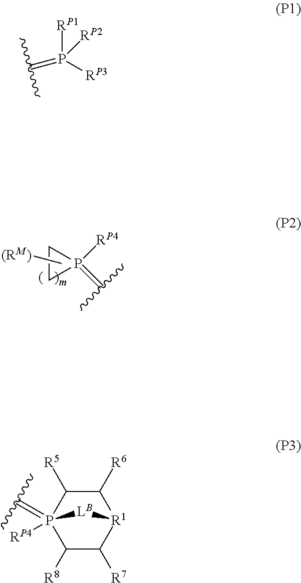
wherein
R^{P1} and R^{P2} are each independently selected from methyl, ethyl, isopropyl and phenyl;
R^{P3} is selected from the group consisting of
methyl and ethyl ,
isopropyl,
cyclopentyl,
t-butyl,
phenyl
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;
wherein Q is a C₅₋₆ heteroaryl group, optionally substituted with one or more groups R^{PA};
R^{P4} is selected from methyl and ethyl;
m is an integer selected from 1, 2 or 3;
R^{M} is one or more optional substituents on the ring independently selected from R^{PC} when attached to a carbon atom adjacent the phosphorus atom, or —OH, —OC₁₋₃alkyl and R^{PC}, when attached to other ring carbons;
—L^{B}— is methylene, ethylene or is absent;
when —L^{B}— is present, R^{P4} is absent and R¹ is selected from N, CH and CR^{PC};
when —L^{B}— is absent, R¹ is selected from the group consisting of: O, NR^{Z}, SO₂, CH₂, CHF, CF₂ and CHR^{PC};
wherein R^{Z} is selected from the group consisting of —H, —C₁₋₃alkyl, —COC₁₋₃alkyl and —SO₂C₁₋₃alkyl;
R⁵ and R⁸ are each independently selected from —H and —R^{PC};
R⁶ and R⁷ are each independently selected from —H and —R^{PC};
wherein R^{PC} is C₁₋₃alkyl, optionally substituted with one or more groups R^{PD};
wherein R^{PA} is selected from the group consisting of: linear or branched C₁₋₆alkyl, C₂₋₆alkenyl or
C₂₋₆alkynyl optionally substituted with one or more groups R^{AL}; —F; —Cl; —Br; —CN; —OH; —
OR^{PE}; —CF₃; —CF₂H; —COR^{PE}; —CH₂OH; —CH₂OR^{PE}; —COOH; —COOR^{PE}; —CONH₂; —CONHR^{PE}; —
CONR^{PE}₂; —OCOR^{PE}; —OCONH₂; —OCONHR^{PE}; —OCONR^{PE}₂; —NH₂; —NHR^{PE}; —NR^{PE}₂; —SO₂NH₂;
—SO₂NHR^{PE}₂; —SO₂NR^{PE}₂; —SO₂R^{PE}; —NHCOH; —NHCOR^{PE}; —NR^{PE}COH and —NR^{PE}COR^{PE};
and R^{PB} is selected from the group consisting of:
linear or branched C₁₋₆alkyl, C₂₋₆alkenyl or C₂₋₆alkynyl optionally substituted with one or more groups R^{AT};
C₃₋₆cycloalkyl, C₄₋₆heterocycloalkyl, C₅₋₆cycloalkenyl or C₅₋₆heterocycloalkenyl optionally substituted with one or more groups R^{AT};
phenyl optionally substituted with one or more groups R^{AR}; and
C₅₋₆heteroaryl optionally substituted with one or more groups R^{AR};
R^{PE} is selected from linear or branched C₁₋₄alkyl optionally substituted with one or more groups R^{PD};
and R^{PD} is selected from the group consisting of: F, OH and OC₁₋₃alkyl;
—L^{A}— is selected from
methylene optionally substituted with one or two groups R^{1A1},
ethylene optionally substituted with one or more groups R^{1A1}, and
a single bond;
R^{A} is selected from the group consisting of
(i) 5-membered heteroaromatic groups containing at least one heteroatom selected from N, O and S optionally C-substituted with one or more groups R^{A1}, and optionally N-substituted with one or more groups R^{NA1},
(ii) 6-membered aromatic groups or heteroaromatic groups containing 1 to 3 N atoms, substituted with one or more groups R^{A1},
(iii) 8- to 10-membered bicyclyl or heterobicyclyl groups with the proviso that R^{A} is not selected from the group (A3) or the groups (X3a) to (X3b) 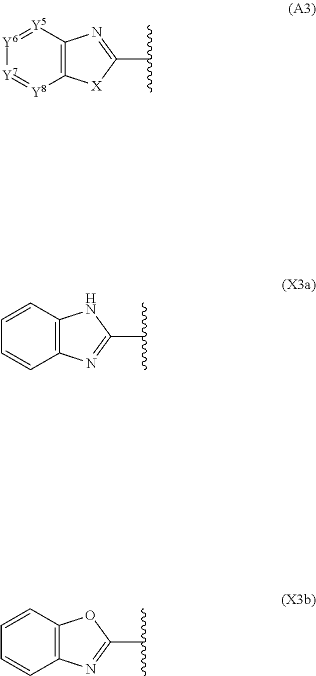
wherein one of Y⁵, Y⁶, Y⁷ and Y⁸ is selected from CH and N, and the others are CH; and
X is independently selected from NH, S and O; and
(iv) the groups (C1) to (C6) 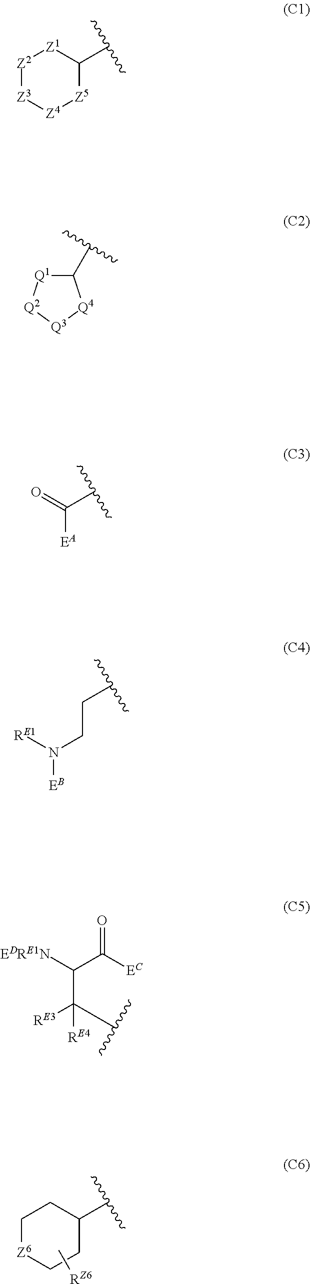
with the proviso that R^{A} is not the group (C3) when L is a single bond;
Z³ is selected from the group consisting of CH₂, CHR^{AL} and CR^{AL}₂;
one of Z¹, Z², Z⁴ and Z⁵ is selected from the group consisting of: CH₂; CHR^{AL}; CR^{AL}₂; O; NH;
NR^{A2}; N(CO—R^{A2}); N(CO—NHR^{A2}); N(SO₂—R^{A2}) and N(CO₂—R^{A4});
the remainder of Z¹, Z², Z⁴ and Z⁵ are independently selected from the group consisting of: CH₂;
CHR^{AL}; CR^{AL}₂ and O;
with the provisos that the ring contains 0 or 1 oxygen atoms, that nitrogen atoms cannot be in a 1,2 or 1,3 relationship to each other, and that when Z¹ or Z⁵ is N, L cannot be a single bond;
one of Q¹ to Q⁴ is selected from the group consisting of: O; NH; NR^{A2}; CH₂; CHR^{AL}; CR^{AL}₂; N—CO—R^{A2}; N—CO—NHR^{A2}; N—SO₂—R^{A2} and N—CO₂—R^{A4};
the remainder of Q¹ to Q⁴ are independently selected from the group consisting of: NH; NR^{A2};
CH₂; CHR^{AL} and CR^{AL}₂;
with the proviso that the ring contains 0 or 1 oxygen atoms, that the ring contains 0 or 1 nitrogen atoms, and that when Q¹ or Q⁴ is N, L cannot be a single bond;
E^{A} is selected from the group consisting of: —O—R^{A2}; —NH—R^{A2}; —NR^{A2}₂; —NR^{EA1}-E^{A1}-COR^{EA2} and —NR^{EA1}-E^{A2}-E^{A3}-COR^{EA2};
wherein E^{A1}, E^{A2} and E^{A3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EA1}— and —COR^{EA2} groups represent terminals of the alpha or pendent functionality of the amino acids respectively;
wherein the amino acid residues Asp and Glu may form amide bonds from either the alpha or pendent carboxylic acid functionality;
when E^{A1} is Pro, R^{EA1} is absent, otherwise R^{EA1} is R^{E1};
when E^{A2} is Pro, R^{EA1} is absent, otherwise R^{EA1} is R^{E1};
wherein the acid functionality of Asp and Glu not forming an amide bond may be present as the corresponding amides or esters selected from —CONH₂, —CONHR^{A2}, —CONR^{A2}R^{E1} and —COOR^{A2};
and the hydroxyl side chain groups of Ser, Thr and Tyr may be present as their corresponding alkoxy or acetate groups selected from —O(C₁₋₃alkyl) and —OCOCH₃; and when E^{A2} and E^{A3} are present and E^{A3} is not Pro the nitrogen of the amide bond between E^{A2} and E^{A3} may be optionally substituted with R^{E1};
R^{EA2} is selected from —OR^{E7}, —NH₂, —NHR^{A2} and —NR^{A2}R^{E1};
R^{E1} is selected from H and linear or branched C₁₋₃alkyl;
E^{B} is selected from: E^{BA}; —CO-E^{B1}-NR^{EA}R^{E2} and —CO-E^{B2}-E^{B3}-NR^{EB}R^{E2};
wherein E^{B1}, E^{B2} and E^{B3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —CO—, —NR^{EA}R^{E2} and —NR^{EB}R^{E2} groups represent terminals of the alpha or pendent functionality of the amino acids;
wherein the amino acid residues Asp and Glu may form amide bonds from either the alpha or pendent carboxylic acid functionality
when E^{B1} is Pro, R^{EA} is absent, otherwise R^{EA} is R^{E1};
when E^{B3} is Pro, R^{EB} is absent, otherwise R^{EB} is R^{E1};
wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃; and when E^{B2} and E^{B3} are present and E^{B2} is not Pro the nitrogen of the amide bond between E^{B2} and E^{B3} may be optionally substituted with R^{E1};
when E^{B} is E^{BA}, R^{E1} and E^{BA} together with the nitrogen atom to which they are attached form a group selected from:
5- or 6-membered saturated heterocyclyl optionally substituted with one or more groups R^{AL}, and
5- or 6-membered heteroaryl optionally substituted with one or more groups R^{A1};
E^{C} is selected from: —OH; —OR^{A2}; —NH₂; NHR^{A2}; NR^{A2}₂ and —NR^{EC1}-E^{C1}-COR^{EC2};
wherein E^{C1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EC1}— and —COR^{EC2} groups represent terminals of the alpha or pendent functionality of the amino acids;
wherein the amino acid residues Asp and Glu may form amide bonds from either the alpha or pendent carboxylic acid functionality;
when E^{C1} is Pro, R^{EC1} is absent, otherwise R^{EC1} is R^{E1};
wherein the acid functionality of Asp and Glu not forming an amide bond may be present as the corresponding amides or esters selected from —CONH₂, —CONHR^{A2}, —CONR^{A2}R^{E1} and —COOR^{A2};
and the hydroxyl side chain groups of Ser, Thr and Tyr may be present as their corresponding alkoxy or acetate groups selected from —O(C₁₋₃alkyl) and —OCOCH₃;
R^{EC2} is selected from —OR^{E9}, —NH₂, —NHR^{A2} and —NR^{A2}R^{E1};
R^{E3} and R^{E4} are independently selected from —H and —CH₃;
when R^{E1} is H and E^{C} is —OC₁₋₃alkyl, —NH₂ or —NHC₁₋₃alkyl, E^{D} is selected from —H, and —CO-E^{D1}-NR^{ED}R^{E6}
otherwise, E^{D} is selected from: —R^{E5}, and —CO-E^{D1}-NR^{ED}R^{E6};
wherein E^{D1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{ED}R^{E6}— and —CO— groups represent terminals of the alpha or pendent functionality of the amino acids;
wherein the amino acid residues Asp and Glu may form amide bonds from either the alpha or pendent carboxylic acid functionality;
wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C₁₋₃alkyl) and —OCOCH₃;
when E^{D1} is Pro, R^{ED} is absent, otherwise R^{ED} is R^{E1};
R^{E2}, R^{E5} and R^{E6} are independently selected from —H and —COCH₃;
R^{E7}, R^{E8} and 8 and R^{E9} are each independently selected from —H and —R^{A2};
Z⁶ is selected from N—CO—R^{RA2}, N—CO—NHR^{A2}, N—SO₂—R^{A2};
R^{Z6} is one or two optional methyl substituents;
R^{A1} is selected from the group consisting of:
linear or branched C₁₋₆alkyl, C₂₋₆alkenyl or C₂₋₆alkynyl optionally substituted with one or more groups R^{AL},
—F, —Cl, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2};
R^{A2} is selected from the group consisting of:
linear or branched C₁₋₆alkyl, C₂₋₆alkenyl or C₂₋₆alkynyl optionally substituted with one or more groups R^{AT}, wherein the alkyl chain is optionally interrupted by one or more atoms selected from O and S;
OC₁₋₆alkyl;
C₃₋₆cycloalkyl, C₄₋₆heterocycloalkyl, C₅₋₆cycloalkenyl or C₅₋₆heterocycloalkenyl optionally substituted with one or more groups R^{AT};
phenyl optionally substituted with one or more groups R^{AR}, and
C₅₋₆heteroaryl optionally substituted with one or more groups R^{AR};
where N is substituted by 2 R^{A2} groups, the N and the R^{A2} groups may together form a N-containing C₅₋₆ heterocycloalkyl group, optionally substituted with one or two groups selected from linear unsubstituted C₁₋₆ alkyl;
R^{NA1} is selected from linear or branched C₁₋₄alkyl;
R^{1A1} is selected from linear or branched unsubstituted C₁₋₃alkyl;
R^{A3} is selected from H and unbranched unsubstituted C₁₋₃alkyl;
R^{A4} is selected from linear or branched unsubstituted C₁₋₄alkyl;
R^{AL} is selected from the group consisting of:
—F, —CN
—OH, —OR^{A2},
—CF₃, —CF₂H.
—COR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2}; and
wherein R^{AR} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{1A1},
—CF₃, —CF₂H,
—COR^{1A1},
—CH₂OH, —CH₂OR^{1A1}, —CHR^{1A1}OH, CHR^{1A1}OR^{1A1}
—COOH, —COOR^{1A1}, —CONH₂, —CONHR^{1A1}, —CONR^{1A1}₂,
—OCOR^{1A1}, —OCONH₂, —OCONHR^{1A1}, —OCONR^{1A1}₂,
—NH₂, —NHR^{1A1}, —NR^{1A1}₂,
—SO₂NH₂, —SO₂NHR^{1A1}₂, —SO₂NR^{1A1}₂,
—SO₂R^{1A1},
—NHCOH, -NHCOR^{1A1}, —NR^{1A1}COH and —NR^{1A1}COR^{1A1};
R^{AT} is selected from the group consisting of
—F, —CN
—OH, —OC₁₋₃alkyl,
—CF₃, —CF₂H,
—COC₁₋₃alkyl,
—COOH, —COOC₁₋₃alkyl, —CONH₂, —CONHC₁₋₃alkyl, —CON(C₁₋₃alkyl)₂,
—OCOC₁₋₃alkyl, —OCONH₂, —OCONHC₁₋₃alkyl, —OCON(C₁₋₃alkyl)₂,
—NH₂, —NHC₁₋₃alkyl, —N(C₁₋₃alky)₂,
—SO₂NH₂, —SO₂NH(C₁₋₃alkyl)₂, —SO₂N(C₁₋₃alkyl)₂,
—SO₂(C₁₋₃alkyl),
—NHCOH, —NHCO(C₁₋₃alkyl), —N(C₁₋₃alkyl)COH and —N(C₁₋₃alkyl)CO(C₁₋₃alkyl).

2. The method according to claim 1, wherein P^{X} is P1, and either R^{P3} is methyl; or R^{P3} is ethyl; or R^{P3} is oxetanyl or tetrahydrofuranyl; or R^{P3} is selected from the group consisting of —CF₃, —CH₂CF₃, —CH₂CF₂H and —CH₂CH₂OR^{PB}, where R^{PB} is a linear or branched C₁₋₆ alkyl; or R^{P3} is —CH₂Q.

3-9. (canceled)

10. The method according to claim 1, wherein P^{X} is P2, and either R^{P4} is methyl; or R^{P4} is ethyl.

11-15. (canceled)

16. The method according to claim 1, wherein P^{X} is P3, —L^{B}— is methylene or ethylene and either R¹ is N; or R¹ is CH; or R¹ is CR^{PC}, wherein R^{PC} is unsubstituted C₁₋₃ alkyl.

17-21. (canceled)

22. The method according to claim 1, wherein P^{X} is selected from the group consisting of:

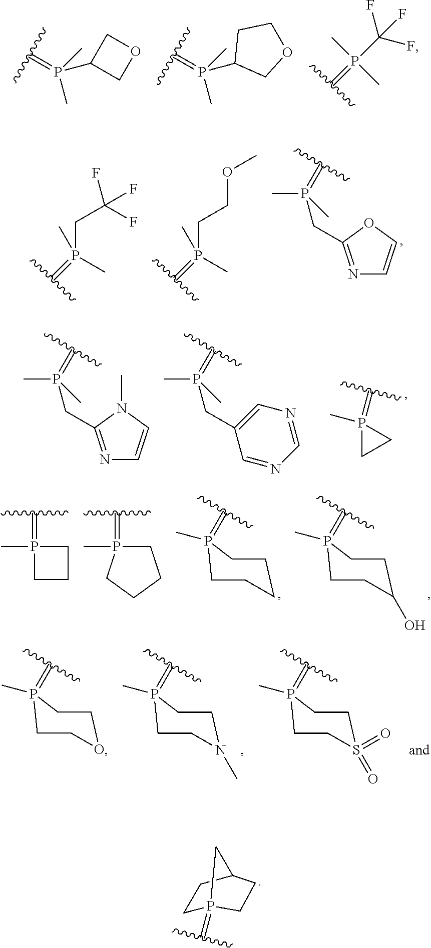

23-29. (canceled)

30. The method according to claim 1, wherein R^{A} is a 5-membered heteroaromatic group containing up to 4 heteroatoms selected from N, O and S, at least one of which being N.

31-46. (canceled)

47. The method according to claim 1, wherein R^{A} is a 6-membered heteroaryl group containing one or two nitrogen atoms, substituted with one or more groups R^{A1}.

48-53. (canceled)

54. The method according to claim 1, wherein R^{A} is a 8- to 10-membered heterobicyclyl group containing one or more heteroatoms independently selected from N, O and S.

55. (canceled)

56. The method according to claim 1, wherein either R^{A} is the group (C1): 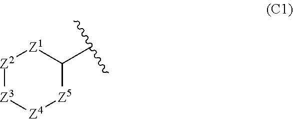
wherein
Z³ is selected from the group consisting of CH₂, CHF and CF₂;
one of Z¹, Z², Z⁴ and Z⁵ is selected from the group consisting of: CH₂; CHR^{AL}; CR^{AL}₂; O; NH;
NR^{A2}; N(CO—R^{A2}); N(CO—NHR^{A2}); N(SO₂—R^{A2}) and N(CO₂—R^{A4}); and
the remainder of Z¹, Z², Z⁴ and Z⁵ are independently selected from the group consisting of: CH₂;
CHR^{AL}; CR^{AL}₂; and O;
with the provisos that the ring contains 0 or 1 oxygen atoms, that nitrogen atoms cannot be in a 1,2 or 1,3 relationship to each other, and that when Z¹ or Z⁵ is N, L cannot be a single bond; or
R^{A} is the group (C₂) 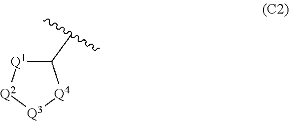
wherein
one of Q¹ to Q⁴ is selected from the group consisting of: O; NH; NR^{A2}; CH ₂; CHR^{AL}; CR^{AL}₂;
N—CO—R^{A2}; N—CO—NHR^{A2}; N—SO₂—R^{A2} and N—CO₂—R^{A4}; and
the remainder of Q¹ to Q⁴ are independently selected from the group consisting of: CH₂; CHR^{AL} and CR^{AL}₂.
with the proviso that the ring contains 0 or 1 oxygen atoms, that the ring contains 0 or 1 nitrogen atoms, and that when Q¹ or Q⁴ is N, L cannot be a single bond.

57-92. (canceled)

93. The method according to claim 1, wherein R^{A} is selected from the group consisting of:

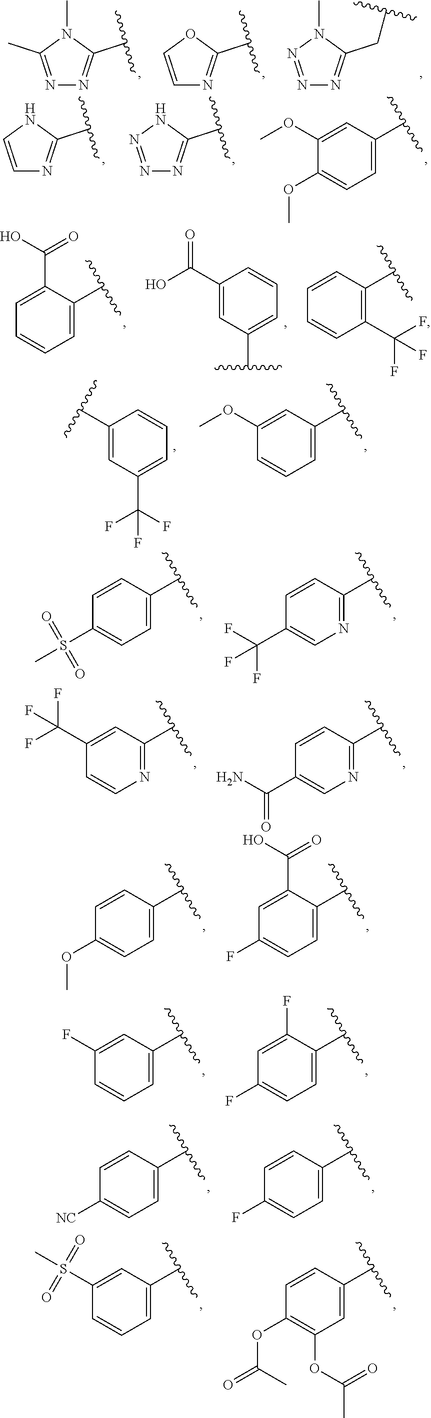

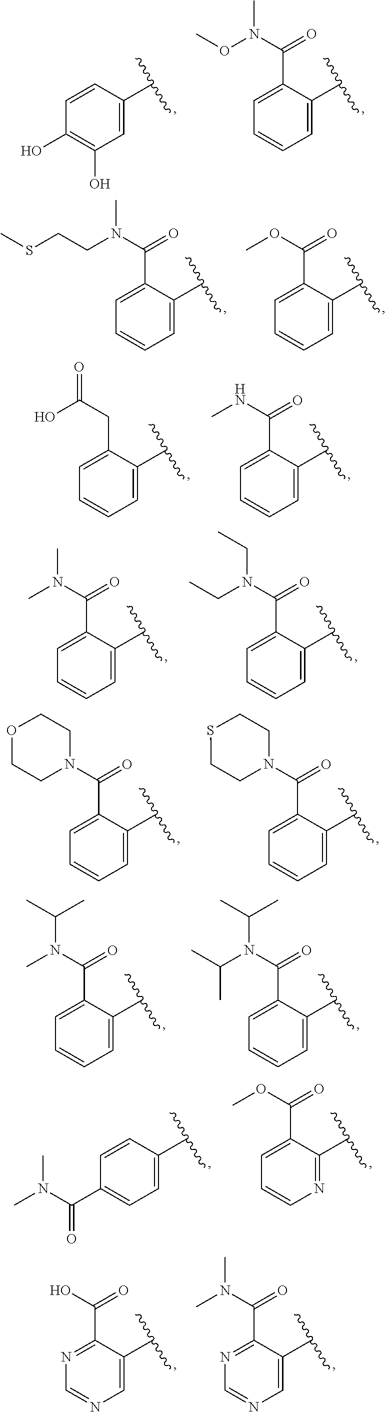

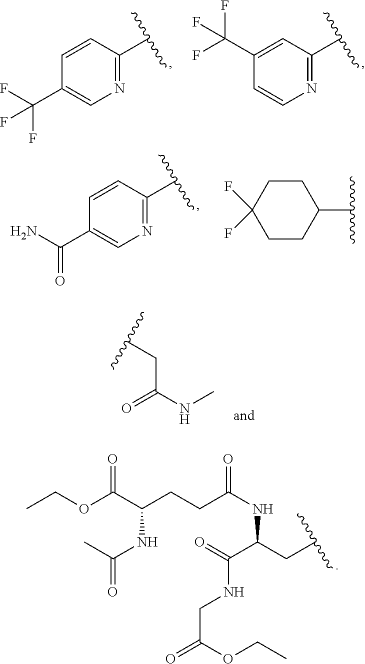

94. The method according to claim 1, wherein R^{A} is the group (C6): 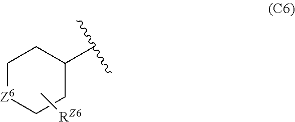
wherein
Z⁶ is selected from N—CO—R^{A2} and N—CO—NHR^{A2}; and
R^{Z6} is one or two optional methyl substituents.

95. A method of preventing or treating a bacterial infection by administering a compound according to Formula (I) or a pharmaceutically acceptable salt, solvate or hydrate thereof: 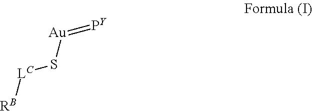
wherein
P^{Y} is independently selected from the group consisting of (P1), (P2) and (P3); 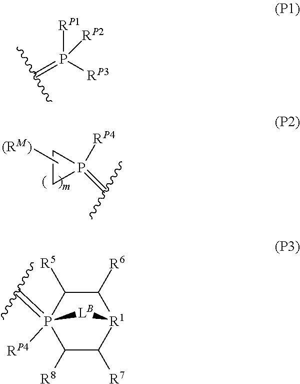
wherein
—L^{C}— is methylene, ethylene or is absent;
R^{P1} and R^{P2} are each independently selected from methyl;
when —L^{C}— is absent R^{P3} is selected from the group consisting of
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;
when —L^{C}— is methylene or ethylene R^{P3} is selected from the group consisting of
methyl and ethyl,
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and -(CH₂)₂Q;
wherein Q is a C₅₋₆ heteroaryl group, optionally substituted with one or more groups R^{PA};
R^{P4} is selected from methyl and ethyl;
m is an integer selected from 1, 2 or 3;
R^{M} is one or more optional substituents on the ring independently selected from
R^{PC} when attached to a carbon atom adjacent the phosphorus atom, or
—OH, —OC₁₋₃alkyl and R^{PC}, when attached to other ring carbons;
—L^{B}— is methylene, ethylene or is absent;
when —L^{B}— is present, R^{P4} is absent and R¹ is selected from N, CH and CR^{PC};
when —L^{B}— is absent, R¹ is selected from the group consisting of
O ,
NR^{Z},
SO₂,
CH₂, CHF, CF₂ and CHR^{P};
wherein R^{Z} is selected from the group consisting of
—H, —C₁₋₃alkyl, —COC₁₋₃alkyl and —SO₂C₁₋₃alkyl;
R⁵ and R⁸ are each independently selected from —H and —R^{PC};
R⁶ and R⁷ are each independently selected from —H and —R^{PC};
wherein R^{PC} is selected from the group consisting of
C₁₋₃alkyl, optionally substituted with one or more groups R^{PD};
wherein R^{PA} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{PE},
—CF₃, —CF₂H,
—COR^{PE},
—CH₂OH, —CH₂OR^{PE},
—COOH, —COOR^{PE}, —CONH₂, —CONHR^{PE}, —CONR^{PE}₂,
—OCOR^{PE}, —OCONH₂, —OCONHR^{PE}, —OCONR^{PE}₂,
—NH₂, —NHR^{PE}, —NR^{PE}₂,
—SO₂NH₂, —SO₂NHR^{PE}₂, —SO₂NR^{PE}₂,
—SO₂R^{PE},
—NHCOH, —NHCOR^{PE}, —NR^{PE}COH and —NR^{PE}COR^{PE};
and R^{PB} is selected from the group consisting of
linear or branched C₁₋₆alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};
R^{PE} is selected from
linear or branched C₁₋₄alkyl optionally substituted with one or more groups R^{PD};
and R^{PD} is selected from the group consisting of
F,
OH and OC₁₋₃alkyl.
R^{B} is independently selected from the groups (A1) to (A5) 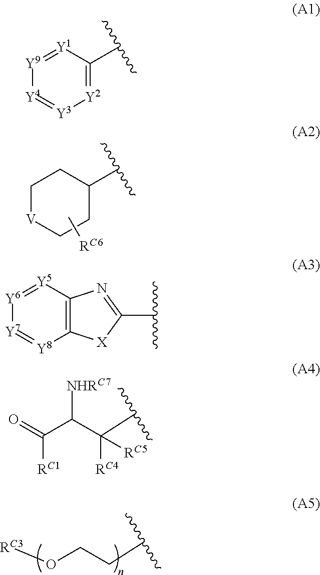
wherein
each of Y¹, Y², Y³, Y⁴ and Y⁹ is independently selected from CH or N; wherein at least three of
Y₁, Y², Y³, Y4 and Y⁹ are independently CH;
V is independently selected from O, CH—OR^{O1}, N—CO—R^{C8}, N—CO—NHR^{C8}, N—SO₂—R^{C8}, N—CO₂—R^{C2} and N—R^{N2};
one of Y⁵, Y⁶, Y⁷ and Y⁸ is selected from CH and N, and the others are CH;
X is independently selected from NH, S and O;
R^{C1} is selected from O—R^{O2} or NHR^{N1};
R^{O1} is selected from H and C₁₋₃ unbranched alkyl;
R^{O2} is selected from H and C¹⁻³ unbranched alkyl;
R^{N1} is selected from H and C¹⁻³ unbranched alkyl;
R^{N2} is C₁₋₃ unbranched alkyl;
R^{C2} and R_{C8} are each independently selected from C¹⁻³ unbranched alkyl and C₃₄ branched alkyl;
R^{C3} is selected from C₁₋₃ unbranched alkyl and C₂H₄CO₂H;
R^{C4} is either H or Me;
R^{C5} is either H or Me;
R^{C6} represents one or two optional methyl substituents;
R^{C7} is selected from —H and —COCH₃; and
n is an integer selected from 2 to 8.

96. The method according to claim 95, wherein P^{Y} is P1, and either R^{P3} is methyl; or R^{P3} is ethyl; or R^{P3} is oxetanyl or tetrahydrofuranyl; or R^{P3} is selected from the group consisting of —CF₃, —CH₂CF₃, —CH₂—CF₂H and —CH₂CH₂OR^{PB} where R^{PB} is a linear or branched C₁₋₆ alkyl; or R^{P3} is —CH₂Q.

97-103. (canceled)

104. The method according to claim 95, wherein P^{Y} is P2, and either R^{P4} is methyl; or R^{P4} is ethyl.

105-109. (canceled)

110. The method according to claim 95, wherein P^{Y} is P3 and either R¹ is N; or R¹ is CH; or R¹ is CR^{PC}, wherein R^{PC} is unsubstituted C₁₋₃ alkyl.

111-115. (canceled)

116. The method according to claim 95, wherein P^{Y} is selected from the group consisting of:

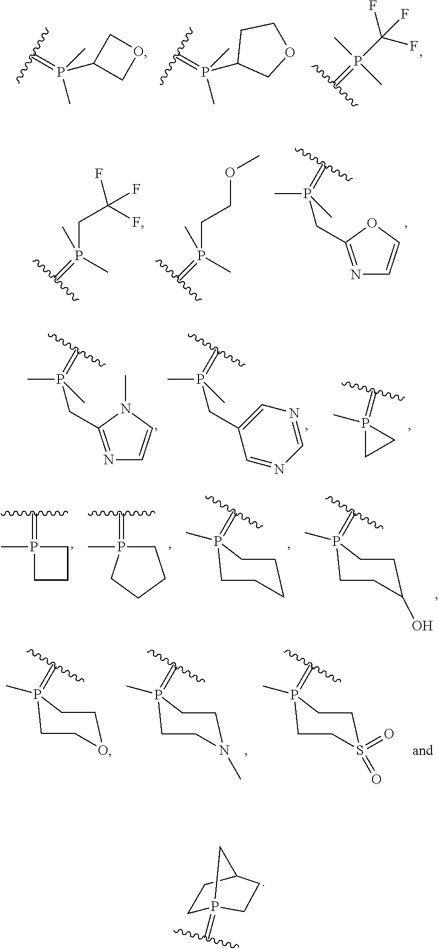

117. The method according to claim 95, wherein R^{B} is A1: 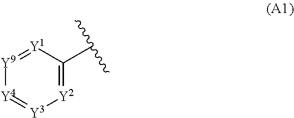
and either one of Y¹, Y², Y³, Y⁴ and Y⁹ is N; or two of Y¹, Y², Y³, Y⁴ and Y⁹ are N; or R^{B} is phenyl.

118-120. (canceled)

121. The method according to claim 95, wherein R^{B} is A2: 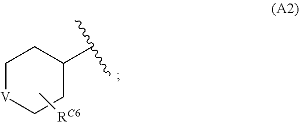
and either V is O; or V is CH—OR^{O1}; or V is N—OC₂—R^{C2}; or V is N—R^{N2}.

122-131. (canceled)

132. The method according to claim 95, wherein R^{B} is A3: 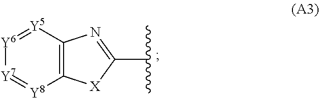
and either X is O and one of Y⁵, Y⁶, Y⁷ and Y⁸ is N; or X is NH and Y⁵, Y⁶, Y⁷ and Y⁸ are CH.

133-134. (canceled)

135. The method according to claim 95, wherein either R^{B} is A4: 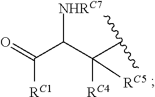
wherein either R^{C1} is O—R^{O2} where R^{O2} is methyl; or R^{C1} is NHR^{N1}, and R^{N1} is H; or
R^{B} is A5:

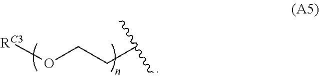

136-145. (canceled)

146. The method according to claim 1, wherein the bacterial infection prevented and/or treated is infection by one or more Gram-positive bacteria; or wherein the bacterial infection prevented and/or treated is infection by one or more Gram-negative bacteria.

147-166. (canceled)

167. A compound of either Formula (I) or Formula (II): 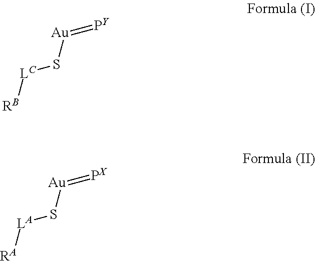
or a pharmaceutically acceptable salt, solvate or hydrate thereof wherein P^{X} is selected from the group consisting of (P1), (P2) and (P3); 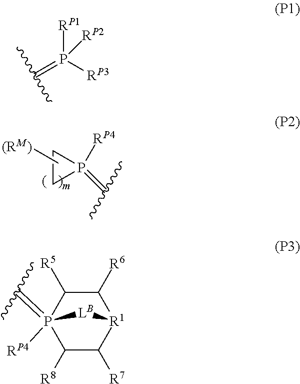
wherein
R^{P1} and R^{P2} are each independently selected from methyl, ethyl, isopropyl and phenyl;
R^{P3} is selected from the group consisting of
methyl and ethyl ,
isopropyl,
cyclopentyl,
t-butyl,
phenyl
4-membered or 5-membered heterocycloalkyl group linked to phosphorus via a carbon atom in the ring, including a single heteroatom independently selected from NR^{Z}, O and S
—CF₃, —CH₂CF₃, —CH₂CF₂H, —CH₂CH₂OR^{PB},
—CH₂Q and —(CH₂)₂Q;
wherein Q is a C₅₋₆ heteroaryl group, optionally substituted with one or more groups R^{PA};
R^{P4} is selected from methyl and ethyl;
m is an integer selected from 1, 2 or 3;
R^{M} is one or more optional substituents on the ring independently selected from
R^{PC} when attached to a carbon atom adjacent the phosphorus atom, or
—OH, —OC¹⁻³alkyl and R^{PC}, when attached to other ring carbons;
—L^{B}— is methylene, ethylene or is absent
when —L^{B}— is present, R^{P4} is absent and R¹ is selected from N, CH and CR^{PC};
when —L^{B}— is absent, R¹ is selected from the group consisting of: O, NR^{Z}, SO₂ CH₂, CHF, CF₂ and CHR^{PC};
wherein R^{Z} is selected from the group consisting of
—H, —C¹⁻³alkyl, —COC¹⁻³alkyl and —SO₂C₁₋₃alkyl;
R⁵ and R⁸ are each independently selected from —H and —R^{PC};
R⁶ and R⁷ are each independently selected from —H and —R^{PC};
wherein R^{PC} is C¹⁻³alkyl, optionally substituted with one or more groups R^{PD};
wherein R^{PA} is selected from the group consisting of: linear or branched C₁₋₆alkyl, C₂₋₆alkenyl or C₂₋₆alkynyl optionally substituted with one or more groups R^{AL}; —F; —Cl; —Br; —CN; —OH; —OR^{PE}; —CF₃; —CF₂H; —COR^{PE}; —CH₂OH; —CH²OR^{PE}; —COOH; —COOR^{PE}; —CONH₂; —CONHR^{PE}; —CONR^{PE}₂; —OCOR^{PE}; —OCONH₂; —OCONHR^{PE}; —OCONR^{PE}₂; —NH₂; —NHR^{PE}; —NR^{PE}₂; —SO₂NH₂; —SO₂NHR^{PE}₂; —SO₂NR^{PE}₂; —SO₂R^{PE}; —NHCOH; —NHCOR^{PE}; —NR^{PE}COH and —NR^{PE}COR^{PE};
and R^{PB} is selected from the group consisting of:
linear or branched C₁₋₆alkyl, C₂₋₆alkenyl or C₂₋₆alkynyl optionally substituted with one or more groups R^{AT};
C₃₋₆cycloalkyl, C₄₋₆heterocycloalkyl, C₅₋₆cycloalkenyl or C₅₋₆heterocycloalkenyl optionally substituted with one or more groups R^{AT};
phenyl optionally substituted with one or more groups R^{AR}; and
C₅₋₆heteroaryl optionally substituted with one or more groups R^{AR};
R^{PE} is selected from linear or branched C₁₋₄alkyl optionally substituted with one or more groups R^{PD};
and R^{PD} is selected from the group consisting of: F, OH and OC¹⁻³alkyl;
—L^{A}— is selected from
methylene optionally substituted with one or two groups R^{1A1},
ethylene optionally substituted with one or more groups R^{1A1}, and
a single bond;
R^{A} is selected from the group consisting of
(i) 5-membered heteroaromatic groups containing at least one heteroatom selected from N, O and S optionally C-substituted with one or more groups R^{A1}, and optionally N-substituted with one or more groups R^{NA1},
(ii) 6-membered aromatic groups or heteroaromatic groups containing 1 to 3 N atoms, substituted with one or more groups ,
(iii) 8- to 10-membered bicyclyl or heterobicyclyl groups with the proviso that R^{A} is not selected from the group (A3) or the groups (X3a) to (X3b) 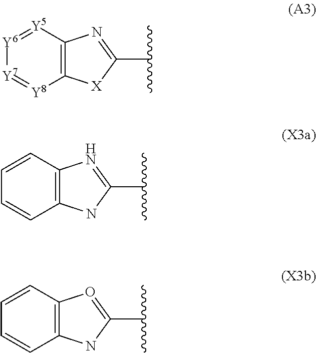
wherein one of Y⁵, Y⁶, Y⁷ and Y⁸ is selected from CH and N, and the others are CH; and
X is independently selected from NH, S and O; and
(iv) the groups (C1) to (C6) 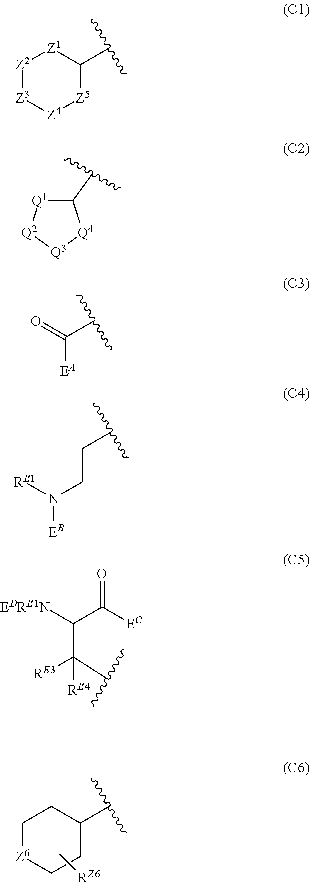
with the proviso that R^{A} is not the group (C3) when L is a single bond;
Z³ is selected from the group consisting of CH₂, CHR^{AL} and CR^{AL}₂;
one of Z¹, Z², Z⁴ and Z⁵ is selected from the group consisting of: CH₂; CHR^{AL}; CR^{AL}₂; O; NH;
NR^{A2}; N(CO—R^{A2}); N(CO—NHR^{A2}); N(SO₂—R^{A2}) and N(CO₂—R^{A4});
the remainder of Z¹, Z², Z⁴ and Z⁵ are independently selected from the group consisting of: CH₂;
CHR^{AL}; CR^{AL}₂ and O;
with the provisos that the ring contains 0 or 1 oxygen atoms, that nitrogen atoms cannot be in a 1,2 or 1,3 relationship to each other, and that when Z¹ or Z⁵ is N, L cannot be a single bond;
one of Q¹ to Q⁴ is selected from the group consisting of: O; NH; NR^{A2}; CH₂; CHR^{AL}; CR^{AL}₂; N—CO—R^{A2}; N—CO—NHR^{A2}; N—SO₂—R^{A2} and N—CO₂—R^{A4};
the remainder of Q¹ to Q⁴ are independently selected from the group consisting of: NH; NR^{A2};
CH₂; CHR^{AL} and CR^{AL}₂;
with the proviso that the ring contains 0 or 1 oxygen atoms, that the ring contains 0 or 1 nitrogen atoms, and that when Q¹ or Q⁴ is N, L cannot be a single bond;
E^{A} is selected from the group consisting of: —O—R_{A2}; —NH—R^{A2}; —NR^{A2}₂; —NR^{EA1}-E^{A1}-COR^{EA2} and
—NR^{EA1}-E^{A2}-E^{A3}-COR^{EA2};
wherein E^{A1}, E^{A2} and E^{A3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EA1}— and —COR^{EA2} groups represent terminals of the alpha or pendent functionality of the amino acids respectively;
wherein the amino acid residues Asp and Glu may form amide bonds from either the alpha or pendent carboxylic acid functionality;
when E^{A1} is Pro, R^{EA1} is absent, otherwise R^{EA1} is R^{E1};
when E^{A2} is Pro, R^{EA1} is absent, otherwise R^{EA1} is R^{E1};
wherein the acid functionality of Asp and Glu not forming an amide bond may be present as the corresponding amides or esters selected from —CONH₂, —CONHR^{A2}, —CONR^{A2}R^{E1} and —COOR^{A2};
and the hydroxyl side chain groups of Ser, Thr and Tyr may be present as their corresponding alkoxy or acetate groups selected from —O(C₁₋₃alkyl) and —OCOCH₃; and when E^{A2} and E^{A3} are present and E^{A3} is not Pro the nitrogen of the amide bond between E^{A2} and E^{A3} may be optionally substituted with R^{E1};
R^{EA2} is selected from —OR_{E7}, —NH₂, —NHR^{A2} and —NR^{A2}R^{E1};
R^{E1} is selected from H and linear or branched C¹⁻³alkyl;
E^{B} is selected from: E^{BA}; —CO-E^{B1}-NR^{EA}R^{E2} and —CO-E^{B2}-E^{B3}-NR^{EB}-R^{E2}.
wherein E^{B1}, E^{B2} and E^{B3} are D- or L-amino acid residues independently selected from Ala, Arg, Asn, Asp, Cys, Gln, Glu, Gly, His, Ile, Leu, Lys, Met, Phe, Pro, Ser, Thr, Trp, Tyr and Val, wherein the —CO—, —NR^{EA}R^{E2} and —NR^{EB}R^{E2} groups represent terminals of the alpha or pendent functionality of the amino acids; p1 wherein the amino acid residues Asp and Glu may form amide bonds from either the alpha or pendent carboxylic acid functionality
when E^{B1} is Pro, R^{EA} is absent, otherwise R^{EA} is R^{E1};
when E^{B3} is Pro, R^{EB} is absent, otherwise R^{EB} is R^{E1};
wherein the acid functionality of Asp and Glu not forming an amide bond may be present as the corresponding amides or esters selected from —CONH₂, —CONHR^{A2}, —CONR^{A2}R^{E1} and —COOR^{A2}; and the hydroxyl side chain groups of Ser, Thr and Tyr may be present as their corresponding alkoxy or acetate groups selected from —O(C¹⁻³alkyl) and —OCOCH₃; and when E^{B2} and E^{B3} are present and E^{B2} is not Pro the nitrogen of the amide bond between E^{B2} and E^{B3} may be optionally substituted with R^{E1};
when E^{B} is E^{BA}, R^{E1} and E^{BA} together with the nitrogen atom to which they are attached form a group selected from:
5- or 6-membered saturated heterocyclyl optionally substituted with one or more groups R^{AL}, and
5- or 6-membered heteroaryl optionally substituted with one or more groups R^{A1};
E^{C} is selected from: —OH; —OR^{A2}; —NH₂; NHR^{A2}; NR^{A2}₂and —NR^{EC1}-E^{C1}-COR^{EC2};
wherein E^{C1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{EC1}— and —COR^{EC2} groups represent terminals of the alpha or pendent functionality of the amino acids;
wherein the amino acid residues Asp and Glu may form amide bonds from either the alpha or pendent carboxylic acid functionality;
when E^{C1} is Pro, R^{EC1} is absent, otherwise R^{EC1} is R^{E1};
wherein the acid functionality of Asp and Glu not forming an amide bond may be present as the corresponding amides or esters selected from —CONH₂, —CONHR^{A2}, —CONR^{A2}R_{E1} and -—COOR^{A2};
and the hydroxyl side chain groups of Ser, Thr and Tyr may be present as their corresponding alkoxy or acetate groups selected from —O—(C₁₋₃alkyl) and —OCOCH₃;
R^{EC2} is selected from —OR^{E9}, —NH₂, —NHR^{A2} and —NR^{A2}R^{E1};
R^{E3} and R^{E4} are independently selected from —H and —CH₃;
when R^{E1} is H and E^{C} is —OC₁₋₃alkyl, —NH₂ or —NHC₁₋₃alkyl, E^{D} is selected from —H, and —CO— E^{D1}-NR^{ED}R^{E6}
otherwise, E^{D} is selected from: —R^{E5}, and —CO-E^{D1}-NR^{ED}R^{E6};
wherein E^{D1} is a D- or L-amino acid residue selected from Ala, Arg, Asn, Asp, Cys, Gln, Glu, Gly, His, Ile, Leu, Lys, Met, Phe, Pro, Ser, Thr, Trp, Tyr and Val, wherein the —NR^{ED}R^{E6}— and —CO— groups represent terminals of the alpha or pendent functionality of the amino acids;
wherein the amino acid residues Asp and Glu may form amide bonds from either the alpha or pendent carboxylic acid functionality;
wherein the acid functionality of Asp and Glu not forming an amide bond may be present as the corresponding amides or esters selected from —CONH₂, —CONHR^{A2}, —CONR^{A2}R^{E1} and —COOR^{A2};
and the hydroxyl side chain groups of Ser, Thr and Tyr may be present as their corresponding alkoxy or acetate groups selected from —O(C₁₋₃alkyl) and —OCOCH₃;
when E^{D1} is Pro, R^{ED} is absent, otherwise R^{ED} is R^{E1};
R^{E2}, R^{E5} and E⁶ are independently selected from —H and —COCH₃;
R^{E7}, R^{E8} and R^{E9} are each independently selected from —H and —R^{A2};
Z⁶ is selected from N—CO—R^{A2}, N—CO—NHR^{A2}, N—SO₂—R^{A2};
R^{Z6} is one or two optional methyl substituents;
R^{A1} is selected from the group consisting of:
linear or branched C₁₋₆alkyl, C₂₋₆alkenyl or C₂₋₆alkynyl optionally substituted with one or more groups R^{AL},
—F, —Cl, —Br, —CN
—OH, —OR^{A2},
—CF₃, —CF₂H,
—COR^{A2},
—CH₂OH, —CH₂OR^{A2},
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH₂, —SO₂NHR^{A2}₂, —SO₂NR^{A2}₂,
—SO₂R^{A2},
—NHCOH, —NHCOR^{A2}, —NR^{A2}COH and —NR^{A2}COR^{A2};
R^{A2} is selected from the group consisting of:
linear or branched C₁₋₆alkyl, C₂₋₆alkenyl or C₂₋₆alkynyl optionally substituted with one or more groups R^{AT}, wherein the alkyl chain is optionally interrupted by one or more atoms selected from O and S;
OC₁₋₆alkyl;
C₃₋₆cycloalkyl, C₄₋₆heterocycloalkyl, C₅₋₆cycloalkenyl or C₅₋₆heterocycloalkenyl optionally substituted with one or more groups R^{AT};
phenyl optionally substituted with one or more groups R^{AR}, and
C₅₋₆heteroaryl optionally substituted with one or more groups R^{AR};
where N is substituted by 2 R^{A2} groups, the N and the R^{A2} groups may together form a N-containing C₅₋₆ heterocycloalkyl group, optionally substituted with one or two groups selected from linear unsubstituted C₁₋₆ alkyl;
R^{NA1} is selected from linear or branched C₁₋₄alkyl;
R^{1A1} is selected from linear or branched unsubstituted C¹⁻³alkyl;
R^{A3} is selected from H and unbranched unsubstituted C¹⁻³alkyl;
R^{A4} is selected from linear or branched unsubstituted C₁₋₄alkyl;
R^{AL} is selected from the group consisting of:
—F, —CN
—OH, —OR^{A2},
—CF₃, —CF₂H,
—CORA²,
—COOH, —COOR^{A2}, —CONH₂, —CONHR^{A2}, —CONR^{A2}₂,
—OCOR^{A2}, —OCONH₂, —OCONHR^{A2}, —OCONR^{A2}₂,
—NH₂, —NHR^{A2}, —NR^{A2}₂,
—SO₂NH, —SO₂NHR^{A2}₂, —SO₂NR^{A2}₂,
—SO₂R^{A2},
—NHCOH, —NHCOR^{A2}, —NR^{A2}COH and —NR^{A2}COR^{A2}; and
wherein R^{AR} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{1A1},
—CF₃, —CF₂H,
—COR^{1A1},
—CH₂OH, —CH₂OR^{1A1}, —CHR^{1A1}OH, CHR^{1A1}OR^{1A1}
—COOH, —COOR^{1A1}, —CONH₂, —CONHR^{1A1}, —CONR^{1A1}₂,
—OCOR^{1A1}, —OCONH₂, —OCONHR^{1A1}, —OCONR^{1A1}₂,
—NH₂, —NHR^{1A1}, —NR^{1A1}₂,
—SO₂NH, —SO₂NHR^{1A1}₂, —SO₂NR^{1A1}₂,
—SO₂R^{1A1},
—NHCOH, —NHCOR^{1A1}, —NR^{1A1}COH and —NR^{1A1}COR^{1A1};
R^{AT} is selected from the group consisting of
—F, —CN
—OH, —OC¹⁻³alkyl,
—CF₃, —CF₂H,
—COC¹⁻³alkyl,
—COOH, —COOC¹⁻³alkyl, —CONH₂, —CONHC₁₋₃alkyl, —CON(C¹⁻³alkyl)₂,
—OCOC₁₋₃alkyl, —OCONH₂, —OCONHC₁₋₃alkyl, —OCON(C¹⁻³alkyl)₂,
—NH₂, —NHC₁₋₃alkyl, —N(C¹⁻³alkyl)₂;
—SO₂NH₂, —SO₂NH(C¹⁻³alkyl)₂, —SO₂N(C¹⁻³alkyl)₂;
—SO₂(C₁₋₃alkyl),
—NHCOH, —NHCO(C₁₋₃alkyl), —N(C₁₋₃alkyl)COH and —N(C₁₋₃alkyl)CO(C₁₋₃alkyl);
with the proviso that when P^{X} is PMe₃ and L^{A} is a single bond, R^{A} is not selected from the groups 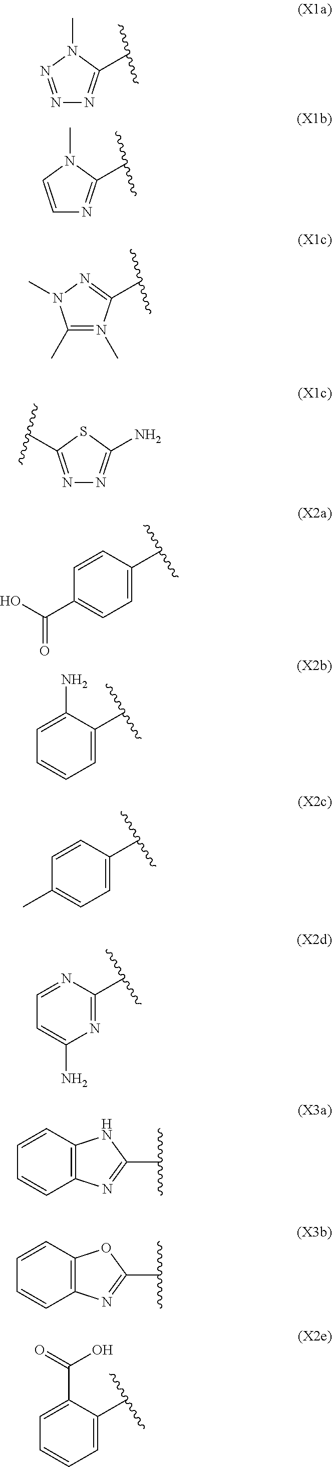
P^{Y} is independently selected from the group consisting of (P1), (P2) and (P3), 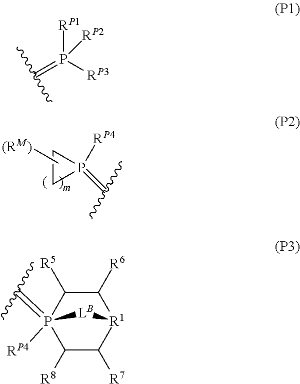
wherein
—L^{C}— is methylene, ethylene or is absent;
R^{P1} and R^{P2} are each independently selected from
methyl;
when —L^{C}— is absent R^{P3} is selected from the group consisting of
4-membered or 5-membered heterocycloalkyl group linked to phosphorus via a carbon atom in the ring, including a single heteroatom independently selected from NR^{Z}, O and S,
—CF₃, —CH₂CF₃, —CH₂CF₂H, —CH₂CH₂OR^{PB},
—CH₂Q and —(CH₂)₂Q;
when —L^{C}— is methylene or ethylene R^{P3} is selected from the group consisting of
methyl and ethyl,
4-membered or 5-membered heterocycloalkyl group linked to phosphorus via a carbon atom in the ring, including a single heteroatom independently selected from NR^{Z}, O and S
—CF₃, —CH₂CF₃, —CH₂CF₂H, —CH₂CH₂OR^{PB},
—CH₂Q and —(CH₂)₂Q;
wherein Q is a C₅₋₆heteroaryl group, optionally substituted with one or more groups R^{PA};
R^{P4} is selected from methyl and ethyl;
m is an integer selected from 1, 2 or 3;
R^{M} is one or more optional substituents on the ring independently selected from
R^{PC} when attached to a carbon atom adjacent the phosphorus atom, or
—OH, —OC¹⁻³alkyl and R^{PC}, when attached to other ring carbons;
—L^{B}— is methylene, ethylene or is absent;
when —L^{B}— is present, R^{P4} is absent and R¹ is selected from N, CH and CR^{PC};
when —L^{B}— is absent, R¹ is selected from the group consisting of
O,
NR^{Z},
SO₂,
CH₂, CHF, CF₂ and CHR^{PC},
wherein R^{Z} is selected from the group consisting of
—H, —C¹⁻³alkyl, —COC¹⁻³alkyl and —SO₂C₁₋₃alkyl;
R⁵ and R⁸ are each independently selected from —H and —R^{PC};
R⁶ and R⁷ are each independently selected from —H and —R^{PC};
wherein R^{PC} is selected from the group consisting of
C₁₋₃alkyl, optionally substituted with one or more groups R^{PD};
wherein R^{PA} is selected from the group consisting of
linear or branched C₁₋₆alkyl, C₂₋₆alkenyl or C₂₋₆alkynyl optionally substituted with one or more groups R^{AL},
—F, —Cl, —Br, —CN
—OH, —OR^{PE},
—CF₃, —CF₂H,
—COR^{PE},
—COOH, —COOR^{PE}, —CONH₂, —CONHR^{PE}, —CONR^{PE}₂,
—OCOR^{PE}, —OCONH₂, —OCONHR^{PE}, —OCONR^{PE}₂,
—NH₂, —NHR^{PE}, —NR^{PE}₂,
—SO₂NH₂, —SO₂NHR^{PE}₂, —SO₂NR^{PE}₂,
—SO₂R^{PE},
—NHCOH, —NHCOR^{PE}, —NR^{PE}COH and —NR^{PE}COR^{PE};
and R^{PB} is selected from the group consisting of
linear or branched C₁₋₆alkyl, C₂₋₆alkenyl or C₂₋₆alkynyl optionally substituted with one or more groups R^{AT},
C₃₋₆cycloalkyl, C₄₋₆heterocycloalkyl, C₅₋₆cycloalkenyl or C₅₋₆heterocycloalkenyl optionally substituted with one or more groups R^{AT},
phenyl optionally substituted with one or more groups R^{AR}, and
C₅₋₆heteroaryl optionally substituted with one or more groups R^{AR};
R^{PE} is selected from
linear or branched C₁₋₄alkyl optionally substituted with one or more groups R^{PD};
and R^{PD} is selected from the group consisting of
—F,
OH and OC¹⁻³alkyl.
R^{B} is independently selected from the groups (A1) to (A5) 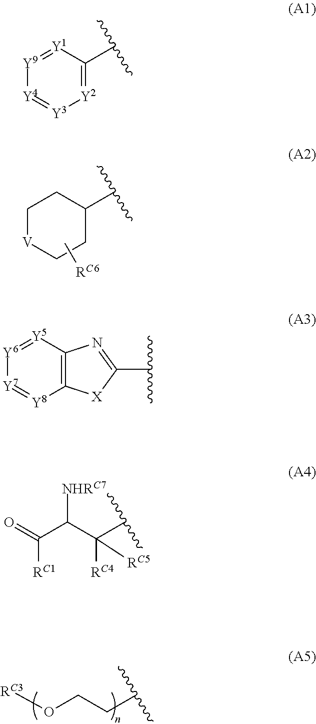
wherein
each of Y¹, Y², Y³, Y⁴ and Y⁹ is independently selected from CH or N; wherein at least three of
Y¹, Y², Y³, Y⁴ and Y⁹ are independently CH;
V is independently selected from O, CH—OR^{O1}, N—CO—R^{C8}, N—CO—NHR^{C8}, N—SO₂—R^{C8}, N—CO₂—R^{C2} and N—R^{N2};
one of Y⁵, Y⁶, Y⁷ and Y⁸ is selected from CH and N, and the others are CH;
X is independently selected from NH, S and O;
R^{O1} is selected from O—R^{O2} or NHR^{N1};
R^{O1} is selected from H and C₁₋₃ unbranched alkyl;
R^{O2} is selected from H and C₁₋₃ unbranched alkyl;
R^{N1} is selected from H and C₁₋₃ unbranched alkyl;
R^{N2} is C₁₋₃ unbranched alkyl;
R^{C2} and R^{C8} are each independently selected from C₁₋₃ unbranched alkyl and C₃₋₄ branched alkyl;
R^{C3} is selected from C₁₋₃ unbranched alkyl and C₂H₄CO₂H;
R^{C4} is either H or Me;
R^{C5} is either H or Me;
R^{C6} represents one or two optional methyl substituents;
R^{C7} is selected from —H and —COCH₃; and
n is an integer selected from 2 to 8

168. (canceled)

169. A pharmaceutical composition comprising a compound according to claim 167 or a pharmaceutically acceptable salt, solvate or hydrate thereof, and a pharmaceutical acceptable diluent or excipient.

170-174. (canceled)
